(19) United States
(12) Patent Application Publication
(10) Pub. No.: US 2024/0092926 A1
(43) Pub. Date: Mar. 21, 2024

(54) IMMUNOMODULATORY ANTIBODIES AND USES THEREOF

(71) Applicant: OncoResponse, Inc., Seattle, WA (US)

(72) Inventors: Jinglei Zhang, Coram, NY (US); Pingping Wang, Farmington, NM (US); Kamal D. Puri, Seattle, WA (US)

(21) Appl. No.: 18/262,173

(22) PCT Filed: Jan. 14, 2022
(86) PCT No.: PCT/US2022/012598

§ 371 Date: Jul. 19, 2023

Related U.S. Application Data
(60) Provisional application No. 63/200,897, filed on Apr. 1, 2021.
(60) Provisional application No. 63/199,732, filed on Jan. 20, 2021.

(51) Int. Cl. C07K 16/28; A61K 45/06; A61P 37/02

(52) U.S. Cl. CPC C07K 16/2896; A61K 45/06; A61P 37/02; C07K 2317/565; C07K 2317/92

(57) ABSTRACT

Provided herein are antibodies and methods of use thereof. The antibodies as disclosed herein bind to CD163⁺ on cells, such as on macrophages. These antibodies can be used in methods of treatment, such as methods of treating cancer and fibrosis.

### CROSS-REFERENCE TO RELATED APPLICATIONS

This application claims the benefit of U.S. Provisional Patent Application Nos. 63/199,732, filed Jan. 20, 2021, and 63/200,897, filed Apr. 1, 2021, each of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Jan. 14, 2022, is named “ONR-006WO_SL.txt” and is 37,676 bytes in size.

### SUMMARY OF THE DISCLOSURE

Provided herein are antibodies, including antigen-binding fragments and other antigen-binding polypeptides, that are useful in the treatment of cancer and fibrosis.

Disclosed herein, in certain embodiments, are antibodies, comprising: (a) a heavy chain variable region (VH) having a sequence at least 80% identical to an amino acid sequence selected from the group consisting of: SEQ ID NO: 29, SEQ ID NO: 31, SEQ ID NO: 33, SEQ ID NO. 35, SEQ ID NO: 37, SEQ ID NO: 39, and SEQ ID NO: 41; and (b) a light chain variable region (VL) having a sequence at least 80% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40; provided that the antibody does not comprise a light chain variable region (VL) having a sequence as set forth in SEQ ID NO: 40 and a heavy chain variable region (VH) having a sequence as set forth in SEQ ID NO. 41. In some embodiments, the light chain variable region (VL) has a sequence at least 85% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the light chain variable region (VL) has a sequence at least 90% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the light chain variable region (VL) has a sequence at least 95% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the light chain variable region (VL) has a sequence at least 990% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the light chain variable region (VL) has a sequence 100% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the heavy chain variable region (VH) has a sequence at least 85% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the heavy chain variable region (VH) has a sequence at least 90% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the heavy chain variable region (VH) has a sequence at least 95% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the heavy chain variable region (VH) has a sequence at least 99% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the heavy chain variable region (VH) has a sequence 100% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41. In some embodiments, the amino acid sequence of the variable heavy chain is 100% identical at CDR H1, CDR H2, CDR H2, and the amino acid sequence of the variable light chain is 100% identical at CDR L1, CDR L2, and CDR L3. In some embodiments, CDR H1 has a sequence as set forth in an amino acid sequence selected from the group consisting of: SEQ ID NO: 4, SEQ ID NO: 16, SEQ ID NO: 19, SEQ ID NO: 22 and SEQ ID NO: 25. In some embodiments, CDR H2 has a sequence as set forth in an amino acid sequence selected from the group consisting of: SEQ ID NO: 5, SEQ ID NO: 17, SEQ ID NO: 20, SEQ ID NO: 23, and SEQ ID NO: 26. In some embodiments, CDR H3 has a sequence as set forth in an amino acid sequence selected from the group consisting of: SEQ ID NO: 6, SEQ ID NO: 18, SEQ ID NO: 21, SEQ ID NO: 24, and SEQ ID NO: 27. In some embodiments, CDR L1 has a sequence as set forth in an amino acid sequence selected from the group consisting of: SEQ ID NO: 1, SEQ ID NO: 7, and SEQ ID NO: 13. In some embodiments, CDR L2 has a sequence as set forth in an amino acid sequence selected from the group consisting of: SEQ ID NO: 2, SEQ ID NO: 9, and SEQ ID NO: 14. In some embodiments, CDR L3 has a sequence as set forth in an amino acid sequence selected from the group consisting of: SEQ ID NO: 3, SEQ ID NO: 8, SEQ ID NO: 10, SEQ ID NO: 11, SEQ ID NO: 12, and SEQ ID NO: 15.

Disclosed herein, in certain embodiments, are antibodies, comprising: (a) a light chain CDR1 having an amino acid sequence at least about 80% identical to an amino acid sequence set forth in the group consisting of: SEQ ID NO: 1, SEQ ID NO: 7, and SEQ ID NO: 13; a light chain CDR2 having an amino acid sequence at least about 80% identical to an amino acid sequence set forth in the group consisting of: SEQ ID NO: 2, SEQ ID NO: 9, and SEQ ID NO: 14; and a light chain CDR3 having an amino acid sequence at least about 80% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 80% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 80% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 80%, identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence at least as set forth in SEQ ID NO: 1, SEQ ID NO: 2, SEQ ID NO: 3, SEQ ID NO: 4, SEQ ID NO: 5, and SEQ ID NO: 6. In some embodiments, CDR L1 has a sequence at least 85% identical to the amino acid sequence selected from the group consisting of: SEQ ID NO: 1, SEQ ID NO: 7, and SEQ ID NO: 13; the CDR L2 has a sequence at least 85% identical to the amino acid sequence selected from the group consisting of: SEQ ID NO: 2, SEQ ID NO: 9, and SEQ ID NO: 14; and the CDR L3 has a sequence at least 85% identical to the amino acid sequence selected from the group consisting of: SEQ ID NO: 3, SEQ ID NO: 8, SEQ ID NO: 10, SEQ ID NO: 11, SEQ ID NO: 12, and SEQ ID NO: 15. In some embodiments. CDR L1 has a sequence at least 90% identical to the amino acid sequence selected from the group consisting of: SEQ ID NO: 1, SEQ ID NO: 7, and SEQ ID NO: 13; the CDR L2 has a sequence at least 90% identical to the amino acid sequence selected from the group consisting of: SEQ ID NO: 2, SEQ ID NO: 9, and SEQ ID NO. 14, and the CDR L3 has a sequence at least 90% identical to the amino acid sequence selected from the group consisting of: SEQ ID NO: 3, SEQ ID NO: 8, SEQ ID NO: 10, SEQ ID NO: 11, SEQ ID NO: 12, and SEQ ID NO: 15. In some embodiments, CDR L1 has a sequence at least 95% identical to the amino acid sequence selected from the group consisting of: SEQ ID NO: 1, SEQ ID NO: 7, and SEQ ID NO: 13; the CDR L2 has a sequence at least 95% identical to the amino acid sequence selected from the group consisting of: SEQ ID NO: 2, SEQ ID NO: 9, and SEQ ID NO: 14; and the CDR L3 has a sequence at least 95% identical to the amino acid sequence selected from the group consisting of: SEQ ID NO: 3, SEQ ID NO: 8, SEQ ID NO: 10, SEQ ID NO: 11, SEQ ID NO: 12, and SEQ ID NO: 15. In some embodiments, CDR L1 has a sequence at least 99% identical to the amino acid sequence selected from the group consisting of: SEQ ID NO: 1, SEQ ID NO: 7, and SEQ ID NO: 13: the CDR L2 has a sequence at least 99% identical to the amino acid sequence selected from the group consisting of: SEQ ID NO: 2, SEQ ID NO: 9, and SEQ ID NO: 14; and the CDR L3 has a sequence at least 99% identical to the amino acid sequence selected from the group consisting of: SEQ ID NO: 3, SEQ ID NO: 8, SEQ ID NO: 10, SEQ ID NO: 11, SEQ ID NO: 12, and SEQ ID NO: 15. In some embodiments, CDR L1 has a sequence 100% identical to the amino acid sequence selected from the group consisting of: SEQ ID NO: 1, SEQ ID NO: 7, and SEQ ID NO: 13; the CDR L2 has a sequence 100% identical to the amino acid sequence selected from the group consisting of: SEQ ID NO: 2, SEQ ID NO: 9, and SEQ ID NO: 14; and the CDR L3 has a sequence at least 100% identical to the amino acid sequence selected from the group consisting of: SEQ ID NO: 3, SEQ ID NO: 8, SEQ ID NO: 10, SEQ ID NO: 11, SEQ ID NO: 12, and SEQ ID NO: 15. In some embodiments, CDR H1 has a sequence at least 85% identical to the amino acid sequence selected from the group consisting of: SEQ ID NO: 4, SEQ ID NO: 16, SEQ ID NO: 19, SEQ ID NO: 22 and SEQ ID NO: 25; the CDR H2 has a sequence at least 85% identical to the amino acid sequence selected from the group consisting of: SEQ ID NO: 5, SEQ ID NO: 17, SEQ ID NO: 20, SEQ ID NO: 23, and SEQ ID NO: 26; and the CDR H3 has a sequence at least 85% identical to the amino acid selected from the group consisting of: SEQ ID NO: 6, SEQ ID NO: 18, SEQ ID NO: 21, SEQ ID NO: 24, and SEQ ID NO: 27. In some embodiments, CDR H1 has a sequence at least 90% identical to the amino acid sequence selected from the group consisting of: SEQ ID NO: 4, SEQ ID NO: 16, SEQ ID NO: 19, SEQ ID NO: 22 and SEQ ID NO: 25; the CDR H2 has a sequence at least 90% identical to the amino acid sequence selected from the group consisting of: SEQ ID NO: 5, SEQ ID NO: 17, SEQ ID NO: 20, SEQ ID NO: 23, and SEQ ID NO: 26; and the CDR H3 has a sequence at least 90% identical to the amino acid selected from the group consisting of: SEQ ID NO: 6, SEQ ID NO: 18, SEQ ID NO: 21, SEQ ID NO: 24, and SEQ ID NO: 27. In some embodiments, CDR H1 has a sequence at least 95% identical to the amino acid sequence selected from the group consisting of: SEQ ID NO: 4, SEQ ID NO: 16, SEQ ID NO: 19, SEQ ID NO: 22 and SEQ ID NO: 25; the CDR H2 has a sequence at least 95% identical to the amino acid sequence selected from the group consisting of: SEQ ID NO: 5, SEQ ID NO: 17, SEQ ID NO: 20, SEQ ID NO: 23, and SEQ ID NO: 26; and the CDR H3 has a sequence at least 95% identical to the amino acid selected from the group consisting of: SEQ ID NO: 6, SEQ ID NO: 18, SEQ ID NO: 21, SEQ ID NO: 24, and SEQ ID NO: 27. In some embodiments, CDR H1 has a sequence at least 99% identical to the amino acid sequence selected from the group consisting of: SEQ ID NO: 4, SEQ ID NO: 16, SEQ ID NO: 19, SEQ ID NO: 22 and SEQ ID NO: 25, the CDR H2 has a sequence at least 99% identical to the amino acid sequence selected from the group consisting of: SEQ ID NO: 5, SEQ ID NO: 17, SEQ ID NO: 20, SEQ ID NO: 23, and SEQ ID NO: 26; and the CDR H3 has a sequence at least 99% identical to the amino acid selected from the group consisting of: SEQ ID NO: 6, SEQ ID NO: 18, SEQ ID NO: 21, SEQ ID NO: 24, and SEQ ID NO: 27. In some embodiments, CDR H1 has a sequence at least 100% identical to the amino acid sequence selected from the group consisting of: SEQ ID NO: 4, SEQ ID NO: 16, SEQ ID NO: 19, SEQ ID NO: 22 and SEQ ID NO: 25; the CDR H2 has a sequence at least 100% identical to the amino acid sequence selected from the group consisting of: SEQ ID NO: 5, SEQ ID NO: 17, SEQ ID NO: 20, SEQ ID NO: 23, and SEQ ID NO: 26; and the CDR H3 has a sequence at least 100% identical to the amino acid selected from the group consisting of: SEQ ID NO: 6, SEQ ID NO: 18, SEQ ID NO: 21, SEQ ID NO: 24, and SEQ ID NO: 27. In some embodiments, the antibody comprises: (a) a heavy chain variable region (VH) having a sequence at least 80% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (b) a light chain variable region (VL) having a sequence at least 80% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the antibody comprises (a) a heavy chain variable region (VH) having a sequence at least 85% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (b) a light chain variable region (VL) having a sequence at least 85% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the antibody comprises (A) a heavy chain variable region (VH) having a sequence at least 90% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (b) a light chain variable region (VL) having a sequence at least 90% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the antibody comprises (a) a heavy chain variable region (VH) having a sequence at least 95% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (b) a light chain variable region (VL) having a sequence at least 95% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40. In some embodiments, the antibody comprises (a) a heavy chain variable region (VH) having a sequence at least 99% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; and (b) a light chain variable region (VL) having a sequence at least 99% identical to an amino acid sequence selected from the group consisting of: SEQ ID NO: 28, SEQ ID NO: 30, SEQ ID NO: 32, SEQ ID NO. 34, SEQ ID NO: 36, SEQ ID NO: 38, and SEQ ID NO: 40. In some embodiments, the antibody comprises (a) a heavy chain variable region (VH) having a sequence 100% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41, and (b) a light chain variable region (VL) having a sequence 100% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40.

In some embodiments, an antibody disclosed herein comprises a human heavy chain constant region or a human light chain constant region. In some embodiments, an antibody disclosed herein comprises the human heavy chain constant region is IgG1 or IgG4 or a fragment thereof. In some embodiments, an antibody disclosed herein binds to an Fc receptor. In some embodiments, the Fc receptor is expressed on the macrophage. In some embodiments, an antibody disclosed herein is an antibody fragment comprising a single heavy chain, a single light chain, Fab, F(ab′), F(ab′)₂, Fd, scFv, a variable heavy domain, a variable light domain, a variable NAR domain, bi-specific scFv, a bi-specific Fab₂, a tri-specific Fab₃ a single chain binding polypeptide, a dAb fragment, or a diabody. In some embodiments, the antibody disclosed herein binds to a dendritic cell (DC), such as, e.g., a myeloid DC (mDCs) (e.g., a CD14⁻HLA-DR⁺CD11c⁺ mDC). In some embodiments, the antibody disclosed herein binds to a classic monocyte (e.g., a CD14⁺HLA-DR⁺CD16⁻ monocyte). In some embodiments, the antibody disclosed herein binds to an intermediate monocyte (e.g., a CD14⁺HLA-DR⁺CD16⁺ monocyte). In some embodiments, the antibody disclosed herein binds to a nonclassic monocyte (e.g., a CD14⁻HLA-DR⁺CD16⁺ monocyte). In some embodiments, an antibody disclosed herein binds to a cancer cell. In some embodiments, the cancer cell is a lymphoma cell. In some embodiments, the lymphoma cell constitutively expresses CD163. In some embodiments, an antibody disclosed herein binds to an immunosuppressive myeloid cell. In some embodiments, the immunosuppressive myeloid cell is in a tumor microenvironment. In some embodiments, the immunosuppressive myeloid cell is a macrophage or a myeloid-derived suppressor cell. In some embodiments, the human macrophage is an M2 macrophage or a M2-like macrophage. In some embodiments, the human macrophage is an M2a, M2b, M2c, or M2d macrophage. In some embodiments, the macrophage is a tumor-associated macrophage. In some embodiments, an antibody disclosed herein binds to a CD163 protein. In some embodiments, the CD163 protein is a glycoform of CD163. In some embodiments, the CD163 protein is a 150 kDa glycoform of CD163. In some embodiments, an antibody disclosed herein does not specifically bind to a 130 kDa glycoform of CD163 expressed by the human macrophage. In some embodiments, the CD163 protein is a component of a cell surface complex comprising at least one other protein expressed by the macrophage. In some embodiments, the at least one other protein is a galectin-1 protein, a LILRB2 protein, a casein kinase II protein, or any combination thereof. In some embodiments, an antibody disclosed herein specifically binds to a CD163 epitope comprising amino acid sequence SEQ ID NO: 42. In some embodiments, an antibody disclosed herein specifically binds to a CD163 epitope comprising amino acid sequence SEQ ID NO: 43. In some embodiments, an antibody disclosed herein specifically binds to a CD163 epitope comprising amino acid sequence SEQ ID NO: 44. In some embodiments, an antibody disclosed herein specifically binds to a CD163 epitope comprising each of amino acid sequence SEQ ID NO: 42, SEQ ID NO: 43, and SEQ ID NO: 44. In some embodiments, an antibody disclosed herein alters expression of at least one marker on the macrophage. In some embodiments, at least one marker on the human macrophage is CD16, CD64, TLR2, or Siglec-15.

In some embodiments, an antibody disclosed herein specifically binds to CD163 with a K_{D} from 0.5 nM to 100 nM. In some embodiments, an antibody disclosed herein specifically binds to CD163 with a K_{D} from 0.5 nM to 50 nM. In some embodiments, an antibody disclosed herein specifically binds to CD163 with a K_{D} from 0.5 nM to 10 nM. In some embodiments, an antibody disclosed herein specifically binds to CD163 with a K_{D} from 0.5 nM to 1.5 nM. In some embodiments, an antibody disclosed herein specifically binds to CD163 with a K_{D} from 0.5 nM to 1.0 nM. In some embodiments, an antibody disclosed herein specifically binds to human M2c macrophages with a K_{D} from 0.5 nM to 100 nM. In some embodiments, an antibody disclosed herein specifically binds to human M2c macrophages with a K_{D} from 0.5 nM to 50 nM. In some embodiments, an antibody disclosed herein specifically binds to human M2c macrophages with a K_{D} from 0.5 nM to 10 nM. In some embodiments, an antibody disclosed herein specifically binds to human M2c macrophages with a K_{D} from 0.5 nM to 1.5 nM. In some embodiments, an antibody disclosed herein specifically binds to human M2c macrophages with a K_{D} from 0.5 nM to 1.0 nM.

Disclosed herein, in certain embodiments, are pharmaceutical compositions comprising (a) an antibody disclosed herein, and (b) at least one pharmaceutically acceptable excipient. In some embodiments, the pharmaceutically acceptable excipients are selected from the group consisting of a stabilizer, buffer, surfactant, filler, solvent, tonicity agent, and antioxidant. In some embodiments, the pharmaceutical composition comprises two or more pharmaceutically acceptable excipients independently selected from the group consisting of stabilizers, buffers, surfactants, fillers, solvents, tonicity agents, and antioxidants.

Disclosed herein, in certain embodiments, are methods of treating a cancer or a fibrotic disease or disorder associated with a presence of M2-macrophages in a subject in need thereof, the method comprising administering to the subject a therapeutically effective amount of an antibody disclosed herein. In some embodiments, binding of the antibody to a macrophage promotes an immune cell function as measured by one or both of the following parameters: activation of a CD4+ T cell, CD8+ T cell, NK cell, or any combination thereof; and proliferation of a CD4+ T cell, CD8+ T cell, NK cell, or any combination thereof. In some embodiments, the activation of a CD4+ T cell, CD8+ T cell, NK cell, or any combination thereof is measured as an enhanced level of IFN-γ, TNF-α, or perforin, or any combination thereof. In some embodiments, binding of the antibody to a macrophage is not cytotoxic to the macrophage. In some embodiments, binding of the antibody to a macrophage results in at least one of the following effects; reduced expression of at least one marker by the macrophage, wherein the at least one marker is CD16, CD64, TLR2, or Siglec-15; internalization of the antibody by the macrophage; secretion of IFN-γ, TNF-α and perforin; activation of a CD4+ T cell, CD8+ T cell, NK cell, or any combination thereof; proliferation of a CD4+ T cell, CD8+ T cell, NK cell, or any combination thereof; and promotion of tumor cell killing in a tumor microenvironment. In some embodiments, binding results in two or more of (a) through (e); three or more of (a) through (e); four or more of (a) through (e); or all of (a) through (e). In some embodiments, binding of the antibody to a macrophage increases an immunostimulatory activity in a tumor microenvironment. In some embodiments, binding of the antibody to a macrophage reduces an immunosuppression activity of the macrophage. In some embodiments, binding of the antibody to a macrophage reduces a tumor promoting activity of the macrophage. In some embodiments, binding of the antibody promotes CD4+ T cell activation, CD4+ T cell proliferation, or both CD4+ T cell activation and proliferation. In some embodiments, binding promotes expression of CD69. ICOS, OX40, PD1, LAG3, CTLA4, or any combination thereof by CD4+ T cells. In some embodiments, binding to the antibody promotes CD8+ T cell activation, CD8+ T cell proliferation, or both CD8+ T cell activation and proliferation. In some embodiments, binding of the antibody promotes expression of ICOS, OX40, PD1, LAG3, CTLA4, or any combination thereof by CD8+ T cells. In some embodiments, binding of the antibody protein promotes cytotoxic lymphocyte-mediated killing of cancer cells. In some embodiments, binding of the antibody promotes NK cell-mediated tumor cell killing. In some embodiments, binding of the antibody promotes expression of IL-2 by T cells. In some embodiments, binding of the antibody increases CD4+ T cells, CD196− T cells, CXCR3+ T cells, CCR4− T cells, or any combination thereof. In some embodiments, binding of the antibody to a macrophage reduces suppression of cytotoxic T cell-mediated killing of tumor cells in the tumor microenvironment. In some embodiments, the cancer is a lung cancer. In some embodiments, the cancer is a lung carcinoma or a lung sarcoma. In some embodiments, the cancer is a lung adenocarcinoma. In some embodiments, any of the methods further comprise administering to the subject an anti-cancer therapeutic. In some embodiments, binding of the antibody to a macrophage reduces the pro-fibrotic function of the macrophage. In some embodiments, the fibrotic disease or disorder is lung fibrosis. In some embodiments, the fibrotic disease or disorder is cardiac fibrosis. In some embodiments, the fibrotic disease or disorder is hepatic fibrosis. In some embodiments, the fibrotic disease or disorder is renal fibrosis. In some embodiments, the fibrotic disease or disorder is retinal fibrosis. In some embodiments, the fibrosis is a primary fibrotic disease or disorder. In some embodiments, the primary fibrotic disease or disorder is idiopathic pulmonary fibrosis (IPF). In some embodiments, the primary fibrotic disease or disorder is hepatic cirrhosis. In some embodiments, the primary fibrotic disease or disorder is systemic sclerosis (SSc). In some embodiments, the primary fibrotic disease or disorder is radiation fibrosis. In some embodiments, the primary fibrotic disease or disorder is scarring associated with a mechanical injury. In some embodiments, the fibrosis is a secondary fibrotic disease. In some embodiments, the secondary fibrotic disease is associated with a disease or disorder selected from the group consisting of: an infection, an autoimmune disease or disorder, cancer, and an inflammatory disease or disorder. In some embodiments, the secondary fibrotic disease is associated with a disease or disorder selected from the group consisting of: atherosclerosis, atrial fibrillation, chronic heart failure, peripheral artery disease, acute coronary syndromes, non-alcoholic fatty liver disease (NAFLD), acute-on-chronic liver failure, acute liver failure, acute kidney injury, acute tubular necrosis, and chronic kidney disease. In some embodiments, the infection is selected from the group consisting of: sepsis, an HIV infection, a SARS-CoV-2 infection, acute viral hepatitis, chronic viral hepatitis, and malaria. In some embodiments, the autoimmune or inflammatory disease or disorder or is selected from the group consisting of: acute lung injury (ALI), acute respiratory distress syndrome (ARDS), hypersensitivity pneumonitis, alcoholic hepatitis, non-alcoholic steatohepatitis, viral hepatitis, sickle cell disease, Type 1 diabetes mellitus, Type 2 diabetes mellitus, Crohn's disease, celiac disease, asthma, sarcoidosis, glomerulonephritis, lupus nephritis, systemic lupus erythematosus, rheumatoid arthritis, Sjögren's Syndrome, scleroderma, cystic fibrosis (CF), graft-versus-host disease, allograft rejection, kidney allograft rejection, sarcoidosis, pulmonary sarcoidosis, hemophagocytic lymphohistiocytosis (HLH), inflammatory arthritis, chronic obstructive pulmonary disease (COPD), asthma, osteoarthritis, fibroids, and multiple sclerosis. In some embodiments, any of the methods further comprise administering to the subject an anti-inflammatory therapy.

Disclosed herein, in certain embodiments, is an antibody according to the foregoing aspects and embodiments for use a medicament.

Disclosed herein, in certain embodiments, is an antibody according to the foregoing aspects and embodiments for use in treating a cancer or a fibrotic disease or disorder associated with a presence of M2-macrophages in a subject in need thereof. In some embodiments, binding of the antibody to a macrophage promotes an immune cell function as measured by one or both of the following parameters: (a) activation of a CD4+ T cell, CD8+ T cell, NK cell, or any combination thereof; and (b) proliferation of a CD4+ T cell, CD8+ T cell, NK cell, or any combination thereof. In some embodiments, the activation of a CD4+ T cell, CD8+ T cell, NK cell, or any combination thereof is measured as an enhanced level of IFN-γ, TNF-α, or perforin, or any combination thereof. In some embodiments, binding of the antibody to a macrophage is not cytotoxic to the macrophage. In some embodiments, binding of the antibody to a macrophage results in at least one of the following effects: (a) reduced expression of at least one marker by the macrophage, wherein the at least one marker is CD16, CD64, TLR2, or Siglec-15; (b) internalization of the antibody by the macrophage; (c) secretion of IFN-γ. TNF-α, and perforin; (d) activation of a CD4+ T cell, CD8+ T cell, NK cell, or any combination thereof; (e) proliferation of a CD4+ T cell, CD8+ T cell, NK cell, or any combination thereof; and (f) promotion of tumor cell killing in a tumor microenvironment. In some embodiments, the binding results in: two or more of (a) through (e); three or more of (a) through (e); four or more of (a) through (e); or all of (a) through (e). In some embodiments, binding of the antibody to a macrophage increases an immunostimulatory activity in a tumor microenvironment. In some embodiments, binding of the antibody to a macrophage reduces an immunosuppression activity of the macrophage. In some embodiments, binding of the antibody to a macrophage reduces a tumor promoting activity of the macrophage. In some embodiments, binding of the antibody promotes CD4+ T cell activation, CD4+ T cell proliferation, or both CD4+ T cell activation and proliferation. In some embodiments, binding promotes expression of CD69, ICOS, OX40, PD1, LAG3, CTLA4, or any combination thereof by CD4+ T cells. In some embodiments, binding to the antibody promotes CD8+ T cell activation, CD8+ T cell proliferation, or both CD8+ T cell activation and proliferation. In some embodiments, binding of the antibody promotes expression of ICOS. OX40, PD1, LAG3, CTLA4, or any combination thereof by CD8+ T cells. In some embodiments, binding of the antibody protein promotes cytotoxic lymphocyte-mediated killing of cancer cells. In some embodiments, binding of the antibody promotes NK cell-mediated tumor cell killing. In some embodiments, binding of the antibody promotes expression of IL-2 by T cells. In some embodiments, binding of the antibody increases CD4+ T cells, CD196− T cells, CXCR3+ T cells, CCR4− T cells, or any combination thereof. In some embodiments, binding of the antibody to a macrophage reduces suppression of cytotoxic T cell-mediated killing of tumor cells in the tumor microenvironment. In some embodiments, the cancer is a lung cancer. In some embodiments, the cancer is a lung carcinoma or a lung sarcoma. In some embodiments, the cancer is a lung adenocarcinoma. In some embodiments, the antibody is formulated for administration to the subject in combination with an anti-cancer therapeutic. In some embodiments, binding of the antibody to a macrophage reduces the pro-fibrotic function of the macrophage. In some embodiments, the fibrotic disease or disorder is lung fibrosis. In some embodiments, the fibrotic disease or disorder is cardiac fibrosis. In some embodiments, the fibrotic disease or disorder is hepatic fibrosis. In some embodiments, the fibrotic disease or disorder is renal fibrosis. In some embodiments, the fibrotic disease or disorder is retinal fibrosis. In some embodiments, the fibrosis is a primary fibrotic disease or disorder. In some embodiments, the primary fibrotic disease or disorder is idiopathic pulmonary fibrosis (IPF). In some embodiments, the primary fibrotic disease or disorder is hepatic cirrhosis. In some embodiments, the primary fibrotic disease or disorder is systemic sclerosis. In some embodiments, the primary fibrotic disease or disorder is radiation fibrosis. In some embodiments, the primary fibrotic disease or disorder is scarring associated with a mechanical injury. In some embodiments, the fibrosis is a secondary fibrotic disease. In some embodiments, the secondary fibrotic disease is associated with a disease or disorder selected from the group consisting of an infection, an autoimmune disease or disorder, cancer, and an inflammatory disease or disorder. In some embodiments, the secondary fibrotic disease is associated with a disease or disorder selected from the group consisting of: atherosclerosis, atrial fibrillation, chronic heart failure, peripheral artery disease, acute coronary syndromes, non-alcoholic fatty liver disease (NAFLD), acute-on-chronic liver failure, acute liver failure, acute kidney injury, acute tubular necrosis, and chronic kidney disease. In some embodiments, the infection is selected from the group consisting of: sepsis, an HIV infection, a SARS-CoV-2 infection, acute viral hepatitis, chronic viral hepatitis, and malaria. In some embodiments, the autoimmune or inflammatory disease or disorder or is selected from the group consisting of: acute lung injury (ALI), acute respiratory distress syndrome (ARDS), hypersensitivity pneumonitis, alcoholic hepatitis, non-alcoholic steatohepatitis, viral hepatitis, sickle cell disease, Type 1 diabetes mellitus, Type 2 diabetes mellitus, Crohn's disease, celiac disease, asthma, sarcoidosis, glomerulonephritis, lupus nephritis, systemic lupus erythematosus, rheumatoid arthritis, Sjögren's Syndrome, scleroderma, cystic fibrosis (CF), graft-versus-host disease, allograft rejection, kidney allograft rejection, sarcoidosis, pulmonary sarcoidosis, hemophagocytic lymphohistiocytosis (HLH), inflammatory arthritis, chronic obstructive pulmonary disease (COPD), asthma, osteoarthritis, fibroids, and multiple sclerosis. In some embodiments, the antibody is formulated for administration to the subject in combination with an anti-inflammatory therapy.

### BRIEF DESCRIPTION OF THE DRAWINGS

FIGS. 1A-1D are a series of plots showing binding of anti-CD163 antibodies to SU-DHL-1 lymphoma cells. Plot showing binding of AB101, human IgG1 isotype control (hIgG1), and a representative variant of AB101 (V3) to wild-type SU-DHL-1 cells (SU-DHL-1-WT)(FIG. 1A) as a function of antibody concentration. Plot showing binding of AB101, human IgG1 isotype control (hIgG1), and V3 to SU-DHL-1 cells co-expressing CD64 (FcγRI; SU-DHL-1-CD64)(FIG. 1B) as a function of antibody concentration. Plot showing binding of AB101, human IgG1 isotype control (hIgG1), and V3 to SU-DHL-1 cells co-expressing CD16 (FcγRIII, SU-DHL-1-CD16)(FIG. 1C) as a function of antibody concentration. Plot showing binding of AB101, human IgG1 isotype control (hIgG1), and V3 to SU-DHL-1 cells co-expressing CD32 (FcγRII; SU-DHL-1-CD32)(FIG. 1D) as a function of antibody concentration.

FIGS. 2A-2F are a series of absorbance plots showing binding of anti-CD163 antibodies to recombinant CD163 protein as measured by ELISA. Absorbance plot showing binding of AB101 parental antibody, V1, and hIgG1 isotype control to recombinant CD163 protein (FIG. 2A). Absorbance plot showing binding of AB101 parental antibody, V2, and hIgG1 isotype control to recombinant CD163 protein (FIG. 2B). Absorbance plot showing binding of AB101 parental antibody, V3, and hIgG1 isotype control to recombinant CD163 protein (FIG. 2C). Absorbance plot showing binding of AB101 parental antibody, V4, and hIgG1 isotype control to recombinant CD163 protein (FIG. 2D). Absorbance plot showing binding of AB101 parental antibody, V5, and hIgG1 isotype control to recombinant CD163 protein (FIG. 2E). Absorbance plot showing binding of AB101 parental antibody, V6, and hIgG1 isotype control to recombinant CD163 protein (FIG. 2F).

FIG. 3 shows binding of anti-CD163 antibodies to M2c macrophages measured as the geometric median fluorescence intensity (gMFI) using fluorescence-activated cell sorting (FACS). Plot shows improved binding of V1 and V4 as compared to the parental AB101 antibody. The hIgG1 isotype control showed no binding in this assay.

FIGS. 4A-4C are a series of bar graphs showing binding of anti-CD163 antibodies to CD163-positive cells (dendritic cells (DCs), classic monocytes, intermediate monocytes, and nonclassic monocytes) isolated from whole blood. Tested antibodies include a commercial available anti-CD163 antibody (R20), parental AB101 antibody, V3, hIgG1 isotype control (IgG1) and murine IgG1 isotype control (mIgG1). V3 showed improved binding to CD163-positive cells in whole blood. Anti-CD163 antibodies were administered at three different concentrations: 1 μg/mL (FIG. 4A), 3 μg/mL (FIG. 4B), and 10 μg/mL (FIG. 4C).

FIGS. 5A-5C are a series of plots showing relief of M2c macrophage-mediated immune suppression in a M2c/CD8+ T cell co-culture. Plot showing restoration of CD8+ T cell counts (proliferation) following treatment with anti-CD163 antibodies at varying concentrations (FIG. 5A). Tested antibodies include parental AB101 antibody, V3, and human IgG1 isotype control. V3 demonstrated about 40-fold higher potency as compared to AB101 in relieving M2c macrophage-mediated suppression of CD8+ T cell proliferation. Plot showing restoration of CD8+ T cell perforin secretion following treatment with anti-CD163 antibodies at varying concentrations (FIG. 5B). V3 demonstrated substantially higher potency than AB101 in relieving M2c macrophage-mediated suppression of CD8+ T cell perforin secretion. Plot showing restoration of CD8+ T cell cytokine (IFNγ) secretion following treatment with anti-CD163 antibodies at varying concentrations (FIG. 5C). V3 demonstrated substantially higher potency than AB101 in relieving M2c macrophage-mediated suppression of CD8+ T cell IFNγ secretion.

### DETAILED DESCRIPTION OF THE DISCLOSURE

Disclosed herein are antibodies that specifically bind to CD163⁺ cells. In some embodiments, the CD163⁺ cells are immunosuppressive myeloid cells. In some embodiments, the CD163⁺ cells are human CD163 expressing myeloid cells. In some embodiments, the CD163⁺ immunosuppressive myeloid cells are human macrophages. In some embodiments, the human CD163⁺ immunosuppressive macrophages are M2 or M2-like macrophages. In some embodiments, the immunosuppressive myeloid cells are myeloid-derived suppressor cells (MDSC). In some embodiments, the human macrophages express high levels of CD163 (CD163^{Hi}). By contrast, other human hematopoietic cells or primary non-immune human cells do not express CD163. For example, M1 and M1-like macrophages do not express CD163.

In some embodiments, macrophages are pulmonary macrophages. In some embodiments, macrophages are alveolar macrophages (AMs). In some embodiments, macrophages are interstitial macrophages.

Monocytes and macrophages exposed to certain inflammatory cytokines or microbe-associated molecular patterns differentiate into pro-inflammatory (M1 or M1-like) or anti-inflammatory M2 or M2-like macrophages. M1 and M2 are classifications used to define macrophages activated in vitro as pro-inflammatory (when classically activated with IFN-γ and lipopolysaccharide) or anti-inflammatory (when alternatively activated with IL-4 or IL-10), respectively, whereas in vivo or ex vivo macrophages with M1 or M2 phenotypes are defined as M1-like or M2-like macrophages. In some embodiments, M2 macrophages are generated by their exposure to certain cytokines. In some embodiments, the M2 macrophages are differentiated by IL-4, IL-10, IL-13, or a combination thereof.

M2-like macrophages have functions and phenotypes corresponding to M2 macrophages and their subtypes. An M2-like macrophage is any in vivo or ex vivo macrophage having a subset of the functional or phenotypic characteristics of M2 macrophages.

In some embodiments, the antibodies of the present disclosure have high avidity and specific binding for immunosuppressive myeloid cells, in particular, macrophages, such as M2 and M2-like macrophages. In some embodiments, the antibodies specifically bind to M2 and M2-like macrophages resident in, infiltrating, or recruited to fibrotic tissues. In some embodiments, the antibodies as disclosed herein do not have appreciable binding to M1 or M1-like macrophages. M1-activated macrophages express transcription factors such as Interferon-Regulatory Factor (IRF5), Nuclear Factor of kappa light polypeptide gene enhancer (NF-κB), Activator-Protein (AP-1) and STAT1. M1 macrophages secrete pro-inflammatory cytokines such as IFN-γ, IL-1, IL-6, IL-12, IL-23 and TNFα. M1 macrophages have functions and phenotypes corresponding to M1 macrophages. An M1-like macrophage is any in vivo or ex vivo macrophage having a subset of the functional or phenotypic characteristics of M1 macrophages.

In some embodiments, the antibodies of the present disclosure do not bind to primary human cells. In some embodiments, the antibodies of the present disclosure do not bind to hematopoietic stem cells, leukocytes, T cells, B cells, NK cells, and granulocytes.

Macrophages generally fall into two categories, M1-like proinflammatory and M2-like immunosuppressive macrophages, based on their functional characteristics, including their relationships to T helper cell (CD4⁺) types Th1 and Th2. M1 macrophages are a model of “classical” and can be generated with IFN-γ with either innate immune activators such as pathogen associated molecular patters (PAMP) (e.g., lipopolysaccharide (LPS)) or damage-associated molecular patterns (DAMPs) as well as inflammatory cytokines (e.g., tumor necrosis factor-alpha (TNF-α). In addition, T cell dependent macrophage activation via the CD40-CD40 ligand pathway induce M1 differentiation. M1 macrophages have pro-inflammatory, bactericidal, and cytotoxic functions. These macrophages promote the antigen-dependent induction of Th1 cells and activation of Th1 and CD8⁺ T cells. In some embodiments, M1-like macrophages are characterized by surface marker expression measured by flow cytometry and have either a CD80⁺CD86⁺CD163^{Lo/-} or CD206^{Lo/-} phenotype. M1 macrophages secrete IL-12, and low level of IL-10 and/or TGF-β.

By contrast, M2-like immunosuppressive macrophages are a model of “alternative” or “non-classical” activation, which can be generated with IL-4 or IL-10 in vitro, are anti-inflammatory and promote wound healing and tissue repair. In some embodiments, M2-like immunosuppressive macrophages are polarized from monocyte-derived macrophages and recruited by factors secreted to tissues in need of wound-healing and/or other forms of tissue repair. M2-like immunosuppressive macrophages are the principal macrophage cell type involved in tissue-regeneration, such as activating and stimulating proliferation of fibroblasts. M2-like macrophages express the surface markers CD15, CD23, CD64, CD68, CD163^{Hi}. CD204^{Hi}, CD206^{Hi}, and/or other M2 macrophage markers determined by flow cytometry. M2 macrophages secrete high levels of IL-10 and TGF-beta1, and low levels of IL-12.

Sub-types of M2 macrophages include M2a, M2b, M2c, and M2d subtypes. M2a macrophages are induced by IL-4 and IL-13, which evokes upregulated expression of CD163, arginase-1, mannose receptor MRC1 (CD206), antigen presentation by MHC II system, and production of IL-10 and TGF-β, leading to tissue regeneration and the inhibition of pro-inflammatory molecules to prevent the inflammatory response. M2b macrophages produce IL-1, IL-6, IL-10, and TNF-α as a response to immune complexes. M2c macrophages are induced by IL-10, transforming growth factor beta (TGF-β) and glucocorticoids exposure, and produce IL-10 and TGF-β, leading to suppression of inflammatory response. M2d subtypes are activated as a response to IL-6 and adenosines.

Macrophage populations can be plastic and differentiate into either M1 or M2 phenotypes depending on the environment (e.g., tissue environment), such as the cytokine environments described above. Macrophage populations can also shift phenotypes during a response. For example, an initial tissue injury or insult (e.g., pathogen, auto-immune, or mechanical mediated injury) can first lead to a pro-inflammatory environment promoting an M1 phenotype then switch to an M2 phenotype during a resolution/rehabilitation phase that can include wound-healing and/or tissue regeneration. However, and without wishing to be bound by theory, excessive wound-healing and/or tissue regeneration mediated by M2 or M2-like macrophages may lead to fibrotic pathogenesis (i.e., “fibrosis”), potentially leading to extensive tissue scarring and organ dysfunction. Such excessive wound-healing and/or tissue regeneration may be the result of chronic injury or insult, such as chronic inflammation.

Accordingly, there remains a need to identify compounds and methods to improve immunotherapeutic treatment of treating fibrosis.

CD163 (scavenger receptor cysteine-rich type 1 protein M130; hemoglobin scavenger receptor) is a cell surface protein which acts as a scavenger receptor for the hemoglobin-haptoglobin complex and protects tissues from free hemoglobin-mediated oxidative damage. Four isoforms of CD163 protein, with molecular weights of 125,451, 125,982, 121,609 and 124,958 Da have been reported. Isoform 1 is the most prevalent isoform of CD163, with a molecular weight of 125,451 Da, and consisting of 1,115 amino acid-residue polypeptide comprising an extracellular domain, a transmembrane segment, and a cytoplasmic tail. The extracellular domain comprises nine cysteine-rich repeat domains. Isoform 1 of CD163 protein has four N-linked glycosylation sites, and in M2 macrophages CD163 protein shows two distinct bands, at ˜150 kDa and ˜130 kDa, in SDS-PAGE under reducing conditions.

CD163 mRNA expression is generally restricted to myeloid cells but is also expressed by certain human cancers. CD163 has also been reported to be a macrophage scavenger receptor and promote immunosuppression. In some embodiments, the interaction of the hemoglobin-haptoglobin complex with CD163 induces the secretion of the immunosuppressive cytokine IL-10 and the expression heme-oxygenase-1 (HO-1). HO-1 produces the anti-inflammatory metabolites Fe²⁺, CO, and bilirubin.

Soluble CD163 occurs in humans via ectodomain shedding and is reported to have anti-inflammatory properties, such as downregulating T-cell responses, including lymphocyte proliferation stimulated by phytohemagglutinin (PHA) or 12-O-tetradecanoylphorbol-13-acetate (TPA).

Antibodies targeting CD163 have been shown to modulate the innate immune response of CD163 expressing macrophages. For example, RM3/1 antibody, an antibody against CD163, is a mouse monoclonal IgG1 (kappa light chain) that was raised against human monocytes. The RM3/1 antibody binds to the cysteine-rich domain 9 of human CD163, reduces LPS-induced TNFα, and enhances IL-10 secretion by macrophages.

### Certain Terminology

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a.” “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20 fold, etc., as well as 1.1, 1.2, 1.3, 1.4, 1.5 fold, etc., 2.1, 2.2, 2.3, 2.4, 2.5 fold, etc., and so forth.

“About” a number, as used herein, refers to range including the number and ranging from 10% below that number to 10% above that number. “About” a range refers to 10% below the lower limit of the range, spanning to 10% above the upper limit of the range.

“Percent identity” and “% identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17): 3389-40), BLASTP, BLASTN, or GCG (Devereux et al., Nucleic Acids Res. 1984 Jan. 11; 12(1 Pt 1):387-95).

As used herein “antibody” refers to a protein that binds an antigen. An antibody often comprises a variable domain and a constant domain in each of a heavy chain and a light chain. Accordingly, most antibodies have a heavy chain variable domain (V_{H}) and a light chain variable domain (V_{L}) that together form the portion of the antibody that binds to the antigen, sometimes referred to as the “antigen receptor.” Within each variable domain are three complementarity-determining regions (CDR), which form loops in the heavy chain variable domain (V_{H}) and light chain variable domain (V_{L}) and contact the surface of the antigen. “Antibody” includes, but is not limited to, polyclonal, monoclonal, monospecific, multispecific (e.g., bispecific antibodies), natural, humanized, human, chimeric, synthetic, recombinant, hybrid, mutated, grafted, antibody fragments (e.g., a portion of a full-length antibody, generally the antigen binding or variable region thereof, e.g., Fab, F(ab′), F(ab′)₂, and Fv fragments), and in vitro-generated antibodies having the antigen-binding activity. The term also includes single chain antibodies, e.g., single chain Fv (sFv or scFv) antibodies, in which a variable heavy and a variable light chain are joined together (directly or through a peptide linker) to form a continuous polypeptide.

As used herein, “complementarity-determining regions,” “CDRs.” and “hypervariable regions” refer to the parts of the variable domains in antibodies that determine the antibodies' binding specificities to their specific antigen. As noted, a single variable region of an antibody polypeptide will typically comprise three CDRs, usually designated CDR1, CDR2, and CDR3. More particularly, a heavy chain variable region may contain CDRs designated H1, H2, and H3; likewise, light chain variable region may contain CDRs L1, L2, and L3. Multiple methods may be used to define a CDR. The current art utilizes various numbering schemes with different definitions of CDR lengths and positions. For example, the Kabat numbering scheme is based on sequence alignment and uses “variability parameter” of a given amino acid position (the number of different amino acids at a given position divided by the frequency of the most occurring amino acid at that position) to predict CDRs. The Chothia numbering scheme, on the other hand, is a structure-based numbering scheme where antibody crystal structures are aligned as define the loop structures as CDRs. The Martin numbering scheme focuses on the structure alignment of different framework regions of unconventional lengths. IMGT numbering scheme is a standardized numbering system based on alignments of sequences from a complete reference gene database including the whole immunoglobulin superfamily. Honneger's numbering scheme (AHo's) is based on structural alignments of the 3D structure of the variable regions and uses structurally conserved Cα positions to deduce framework and CDR lengths. One of skill in the art will note that the definition of a CDR will vary based on the method used. Any method of defining a CDR is contemplated with the sequences disclosed herein.

The terms “recipient,” “individual,” “subject,” “host,” and “patient,” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys, etc. In some embodiments, the mammal is a human.

As used herein, the terms “treatment,” “treating,” and the like, in some cases, refer to administering an agent, or carrying out a procedure, for the purposes of obtaining an effect. In some embodiments, the effect is prophylactic in terms of completely or partially preventing a disease or symptom thereof and/or is therapeutic in terms of effecting a partial or complete cure for a disease and/or symptoms of the disease. “Treatment,” as used herein, includes treatment of a disease or disorder (e.g., fibrosis) in a mammal, particularly in a human, and includes: (a) preventing the disease or a symptom of a disease from occurring in a subject which is predisposed to the disease but has not yet been diagnosed as having it (e.g., including diseases that is associated with or caused by a primary disease; (b) inhibiting the disease, i.e., arresting its development; and (c) relieving the disease. i.e., causing regression of the disease. In some embodiments, treating refers to any indicia of success in the treatment or amelioration or prevention of fibrosis, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters, including the results of an examination by a physician. Accordingly, the term “treating” includes the administration of the compounds or agents of the present disclosure to prevent or delay, to alleviate, or to arrest or inhibit development of the symptoms or conditions associated with diseases (e.g., fibrosis). The term “therapeutic effect” refers to the reduction, elimination, or prevention of the disease, symptoms of the disease, or side effects of the disease in the subject. A subject is “treated” for a disease or disorder if, after receiving a therapeutic amount of an antibody of the present disclosure, the patient shows observable and/or measurable change in a parameter or symptom of the disease or disorder.

In some embodiments, “inducing a response” refers to the alleviation or reduction of signs or symptoms of illness in a subject, and specifically includes, without limitation, prolongation of survival.

The term “avidity” refers to the resistance of a complex of two or more agents to dissociation after dilution.

In some embodiments, antibody “effector functions” refers to those biological activities attributable to the Fc region (a native sequence Fc region or amino acid sequence variant Fc region) of an antibody and vary with the antibody isotype.

“Fc receptor” or “FcR” refers to a receptor that binds to the Fc region of an antibody.

“Human effector cells” as used herein refers to leukocytes that express one or more FcRs and perform effector functions. For example, the cells express at least FcγRIII and perform an ADCC effector function. Examples of human leukocytes that mediate ADCC include, but are not limited to, peripheral blood mononuclear cells (PBMC), NK cells, monocytes, macrophages, cytotoxic T cells, and neutrophils.

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass) that are bound to their cognate antigen. To assess complement activation, a CDC assay, for example, is performed.

An antibody that “internalizes” is one that is taken up by (i.e., enters) the cell upon binding to an antigen on a mammalian cell (e.g., a cell surface polypeptide or receptor). The internalizing antibody comprises antibody fragments, human or chimeric antibody, and antibody conjugates. In some cases, internalization of an antibody (e.g., such as disclosed herein) alter the biology of the cell, causing it to change its function.

An “antigen-binding domain,” “antigen-binding region,” or “antigen-binding site” is a portion of an antibody that contains amino acid residues (or other moieties) that interact with an antigen and contribute to the antibody's specificity and affinity for the antigen. For an antibody that specifically binds to its antigen, this will include at least part of at least one of its CDR domains.

The antigen-binding region of an antibody is referred to as a “paratope,” which binds to an antigenic determinant, the “epitope” of an antigen, that is, a portion of the antigen molecule that can be bound by an antibody. In some embodiments, an antigen substance has one or more portions that are recognizable by antibodies, i.e., more than one epitope, and thus a single antigen substance is specifically bound by different antibodies each having specificity for a different epitope. In some embodiments, an epitope comprises non-contiguous portions of the antigen. For example, in a polypeptide, amino acid residues that are not contiguous in the polypeptide's primary sequence but that, in the context of the polypeptide's tertiary and quaternary structure, are near enough to each other to be bound by an antigen-binding protein, constitutes an epitope.

An “antibody fragment” comprises a portion of an intact antibody. In some embodiments, the antibody fragment comprises an antigen-binding or variable region of the intact antibody.

The terms “antigen-binding portion of an antibody,” “antigen-binding fragment,” “antigen-binding domain,” “antibody fragment” are used interchangeably herein to refer to one or more fragments of an antibody that retain the ability to specifically bind to the antigen. Non-limiting examples of antibody fragments included within such terms include, but are not limited to, (i) a Fab fragment, a monovalent fragment consisting of the V_{L}, V_{H}, C_{L}, and C_{H} domains; (ii) a F(ab′)₂ fragment, a bivalent fragment containing two Fab fragments linked by a disulfide bridge at the hinge region; (iii) a Fd fragment consisting of the V_{H} and C_{H} domains; (iv) a Fv fragment containing the V_{L} and V_{H} domains of a single arm of an antibody, (v) a dAb fragment (Ward et al., Nature 341(6242):544-6 (1989)), containing a V_{H} domain; and (vi) an isolated CDR. Also included are “one-half” antibodies comprising a single heavy chain and a single light chain. Other forms of single chain antibodies, such as diabodies are also encompassed herein.

A “functional antibody fragment” as used herein refers in context to an antibody fragment that not only binds the antibody's antigen, but also possesses a functional attribute that characterizes the intact antibody. For example, if an antibody depends for a function on possessing a Fc domain that enables an effector function, such as ADCC, a functional fragment would possess such function. It is hypothesized that antibodies of the disclosure are effective in modulating the functional state of macrophages, such as tissue-resident or infiltrating macrophages, or reorienting or dampening the M2-status macrophages, when they comprise an Fc portion that binds to a macrophage Fc receptor, such as CD16 (FcγRIIIa) or CD64 (FcγRI) in some embodiments.

The phrase “functional fragment or analog” of an antibody is a compound having qualitative biological activity in common with a full-length antibody. For example, a functional fragment or analog of an anti-IgE antibody is one that binds to an IgE immunoglobulin to prevent or substantially reduce the ability of such molecule from having the ability to bind to the high affinity receptor, FcγRI.

An “antigen-binding protein” is a protein comprising a portion that comprises an antigen-binding portion of an antibody, optionally also including a scaffold or framework portion that allows the antigen-binding portion to adopt a conformation that promotes binding of the antigen-binding protein to the antigen.

An “intact” antibody is one that comprises an antigen-binding site as well as a C_{L} and at least heavy chain constant domains, C_{H}1, C_{H}2, and C_{H}3. In some embodiments, the constant domains are native sequence constant domains (e.g., human native sequence constant domains) or amino acid sequence variant thereof.

The term “recombinant antibody” as used herein refers to an antibody comprising an antigen-binding domain of a first antibody, such as, for example, a CDR, a V_{H} region, or an intact light chain, and a domain from one or more other antibodies or proteins. Chimeric, hybrid, and humanized antibodies are examples of recombinant antibodies.

A “CDR-grafted antibody” is an antibody comprising one or more CDRs derived from an antibody of one species or isotype and the framework of another antibody of the same or different species or isotype.

The term “human antibody” includes all antibodies that have one or more variable and constant regions derived from human immunoglobulin sequences. In one embodiment, all of the variable and constant domains of the antibody are derived from human immunoglobulin sequences (referred to as a “fully human antibody”).

As used herein, the term “affinity” refers to the equilibrium constant for the reversible binding of two agents and is expressed as the equilibrium dissociation constant, K_{D}. In one embodiment, the antibodies or antigen-binding fragments thereof exhibit binding affinity as measured by K_{D} for CD163 in the range of 10⁻⁶ M or less, or ranging down to 10⁻¹⁶ M or lower, (e.g., about 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶ M or less). In certain embodiments, antibodies as describe herein specifically bind to a human CD163 (huCD163) polypeptide with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M.

The terms “preferentially binds” or “specifically binds” mean that the antibodies or fragments thereof bind to an epitope with greater affinity than it binds unrelated amino acid sequences, and, if cross-reactive to other polypeptides containing the epitope, are not toxic at the levels at which they are formulated for administration to human use. In some embodiments, such affinity is at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody or fragment thereof for unrelated amino acid sequences.

The term “specific” refers to a situation in which an antibody will preferentially bind to molecules other than the antigen containing the epitope recognized by the antibody. The term is also applicable where for example, an antigen-binding domain is specific for a particular epitope which is carried by a number of antigens, in which case the antibody or antigen-binding fragment thereof carrying the antigen-binding domain will be able to bind to the various antigens carrying the epitope.

As used herein, an antibody is said to be “immunospecific” or “specific” for, or to “specifically bind” to, an antigen if that antibody reacts at a detectable level with the antigen, preferably with an affinity constant (association constant). KA, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁹ M⁻¹.

The term “monospecific,” as used herein, refers to an antibody composition that contains an antibody that displays a preferential affinity for one particular epitope. In some embodiments, monospecific antibody preparations are made up of about 10%, 20%, 30%, 40%, 50%, 60%, 70%, 75%, 80%, 85%, 90%, 95%, 97%, 99%, or 99.9% antibody having specific binding activity for the particular antigen.

The term “polypeptide” is used in its conventional meaning, i.e., as a sequence of amino acids. The polypeptides are not limited to a specific length of the product. Peptides, oligopeptides, and proteins are included within the definition of polypeptide, and such terms are used interchangeably herein unless specifically indicated otherwise. This term also does not refer to or exclude post-expression modifications of the polypeptide, for example, glycosylations, acetylations, phosphorylations, and the like, as well as other modifications known in the art, both naturally occurring and non-naturally occurring. In some embodiments, a polypeptide is an entire protein, or a subsequence thereof. Particular polypeptides of interest in the context of the antibodies of this disclosure are amino acid subsequences comprising CDRs and being capable of binding human M2 macrophages or CD163 protein expressed by such cells.

As used herein, “substantially pure,” and “substantially free” refer to a solution or suspension containing less than, for example, about 20% or less extraneous material, about 10% or less extraneous material, about 5% or less extraneous material, about 4% or less extraneous material, about 3% or less extraneous material, about 2% or less extraneous material, or about 1% or less extraneous material.

The term “isolated” refers to a protein (e.g., an antibody), nucleic acid, or other substance that is substantially free of other cellular material and/or chemicals. In some embodiments, the antibodies, or antigen-binding fragments thereof, and nucleic acids of the disclosure are isolated. In some embodiments, the antibodies, or antigen-binding fragments thereof, and nucleic acids of the disclosure are substantially pure.

When applied to polypeptides, “isolated” generally means a polypeptide that has been separated from other proteins and nucleic acids with which it naturally occurs. Preferably, the polypeptide is also separated from substances such as antibodies or gel matrices (polyacrylamide) which are used to purify it. In some cases, the term means a polypeptide or a portion thereof which, by virtue of its origin or manipulation: (i) is present in a host cell as the expression product of a portion of an expression vector; or (ii) is linked to a protein or other chemical moiety other than that to which it is linked in nature, or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically; or (ii) expressed in a host cell and purified away from associated and contaminating proteins.

The term “effective amount” as used herein, refers to that amount of an antibody, or an antigen-binding portion thereof as described herein, that is sufficient to induce a response, e.g., to effect treatment, prognosis, or diagnosis of a disease associated with macrophage activity, as described herein, when administered to a subject. Therapeutically effective amounts of antibodies provided herein, when used alone or in combination, will vary depending upon the relative activity of the antibodies and combinations (e.g., in treating/reducing/ameliorating fibrosis) and depending upon the subject and disease condition being treated, the weight and age of the subject, the severity of the disease condition, the manner of administration, and the like, which, in some cases, are readily determined by one of ordinary skill in the art.

The term “therapeutically effective amount” generally refers to an amount of an antibody or a drug effective to “treat” a disease or disorder in a subject or mammal. In some embodiments, a composition described herein is administered to a subject in an amount that is effective for producing some desired therapeutic effect by inhibiting a disease or disorder as described herein at a reasonable benefit/risk ratio applicable to any medical treatment. A therapeutically effective amount is an amount that achieves at least partially a desired therapeutic or prophylactic effect in an organ or tissue. The amount of an antibody necessary to bring about prevention and/or therapeutic treatment of a disease or disorder is not fixed per se. In some embodiments, the amount of the antibody administered varies with the type of disease, extensiveness of the disease, and size of the mammal suffering from the disease or disorder. When used in conjunction with therapeutic methods involving administration of a therapeutic agent after the subject presents symptoms of a disease or disorder, the term “therapeutically effective” means that, after treatment, one or more signs or symptoms of the disease or disorder is ameliorated or eliminated.

An effective response of the present disclosure is achieved when the subject experiences partial or total alleviation or reduction of signs or symptoms of illness and, in the case of the treatment of fibrosis, specifically includes, without limitation, amelioration of symptoms, prolongation of progression, cure, remission, prolongation of survival, or other objective responses. In some embodiments, the expected progression-free survival times are measured in months to years, depending on prognostic factors including the number of relapses, stage of disease, and other factors. Prolonging survival includes without limitation times of at least 1 month (mo.), about at least 2 mos., about at least 3 mos., about at least 4 mos., about at least 6 mos., about at least 1 year, about at least 2 years, about at least 3 years, etc. Overall survival is also measured, for example, in months to years. Alternatively, an effective response, in some embodiments, is that a subject's symptoms remain static. Further indications of treatment of indications are described in more detail below.

In some embodiments, administration of a therapeutic agent in a prophylactic method occurs prior to the manifestation of symptoms of an undesired disease or disorder, such that the disease or disorder is prevented or, alternatively, delayed in its progression. Thus, when used in conjunction with prophylactic methods, the term “therapeutically effective” means that, after treatment, a smaller number of subjects (on average) develop the undesired disease or disorder or progress in severity of symptoms.

The phrase “pharmaceutically acceptable” refers to molecular entities and compositions that are physiologically tolerable and do not typically produce an allergic or similar untoward reaction, such as gastric upset, dizziness, and the like, when administered to a human.

The term “contacting” is defined herein as a means of bringing a composition as provided herein in physical proximity with a cell, organ, tissue, or fluid as described herein.

As used herein, the term “primary fibrotic disease” means a disease where the pathology of the disease is driven by fibrosis. Exemplary primary fibrotic diseases include, but are not limited to cystic fibrosis, idiopathic pulmonary fibrosis, hepatic cirrhosis, systemic sclerosis (SSc), sclerodermatous graft vs. host disease (GvHD), nephrogenic systemic fibrosis, and radiation fibrosis.

As used here, the term “secondary fibrotic disease” means a disease in which fibrosis occurs as a sequela of a non-fibrotic disease. In certain instances, the secondary fibrosis is fibrosis associated with cancer. Another example of a secondary fibrotic disease is pulmonary fibrosis associated with paracoccidioidomycosis.

### Antibodies

Disclosed herein, in certain embodiments, are antibodies that specifically bind to a CD163 protein expressed on human CD163⁺ cell. In some embodiments, the CD163⁺ cell is an immunosuppressive myeloid cell. In some embodiments, the immunosuppressive myeloid cell is human macrophage. In some embodiments, the binding of an antibody disclosed herein alters expression of at least one marker on the human macrophage.

In some embodiments, an antibody disclosed herein binds to a huCD163 protein expressed on a human M2 or M2-like immunosuppressive macrophage. In some embodiments, the antibody specifically binds to a CD163 protein that is an approximately 140 kDa glycoform of huCD163. In some embodiments, the antibody specifically binds to extracellular domain 3 of huCD163. In some embodiments, the antibody specifically binds to extracellular domain 4 of huCD163. In some embodiments, the antibody specifically binds to extracellular domain 3 and extracellular domain 4 of huCD163. In some embodiments, the antibody specifically binds to huCD163, resulting in a conformational change of huCD163. In some embodiments, the conformational change to huCD163 exposes extracellular domains 2, 5, and 9 of huCD163. In some embodiments, the antibody does not specifically bind a lower molecular weight (˜115 kDa) glycoform of huCD163.

In some embodiments, an antibody disclosed herein binds to a dendritic cell (DC), such as, e.g., a myeloid DC (mDC)(e.g., a CD14⁻HLA-DR⁺CD11c⁺ mDC). In some embodiments, an antibody disclosed herein binds to a classic monocyte (e.g., a CD14⁺HLA-DR⁺CD16⁻ monocyte).

In some embodiments, an antibody disclosed herein binds to an intermediate monocyte (e.g., a CD14⁺HLA-DR⁺CD16⁺ monocyte). In some embodiments, an antibody disclosed herein binds to a nonclassic monocyte (e.g., a CD14⁻HLA-DR⁺CD16⁺ monocyte). In some embodiments, an antibody disclosed herein binds to a cancer cell. In some embodiments, the cancer cell is a lymphoma cell. In some embodiments, the lymphoma cell constitutively expresses CD163.

In some embodiments, an antibody disclosed herein binds to a human CD163⁺ immunosuppressive myeloid cell and causes an alteration in the expression of certain cell markers that characterize a M2 or M2-like immunosuppressive macrophage (such as a M2c macrophage), indicating a functional differentiation of the macrophages to a non- or less immunosuppressive as well as a less pro-fibrotic state. In some embodiments, an antibody disclosed herein binds to a M2 or M2-like immunosuppressive macrophage and causes a decrease in the expression of certain cell markers that characterize a M2 or M2-like macrophage, indicating a functional differentiation of the macrophages to an altered differentiation state. In some embodiments, an antibody disclosed herein reduces expression of one or more of CD16. CD64, TLR2, and Siglec-15 by the CD163⁺ immunosuppressive myeloid cell.

In some embodiments, the binding of an antibody disclosed herein to a CD163⁺ immunosuppressive myeloid cells results in a functional change in the CD163⁺ immunosuppressive myeloid cell. In some embodiments, the binding of the antibody disclosed herein to the CD163⁺ immunosuppressive myeloid cell results in changes in marker expression in the M2 or M2-like immunosuppressive macrophages.

In some embodiments, an antibody of the present disclosure reduces immunosuppression caused by tumor-associated macrophages in tumor microenvironments. In some embodiments, a reduction in immunosuppression by tumor-associated macrophages in the tumor microenvironment corresponds to an increase in immunostimulation, e.g., production of promotion of T cell activation, T cell proliferation, NK cell activation, NK cell proliferation, or any combination thereof. In some embodiments, T cell activation and/or NK cell activation results in increased production of IFN-γ, TNF-α, perforin, or a combination thereof by T cells and/or NK cells. In some embodiments, the antibodies of the present disclosure increase immunostimulation, e.g., production of promotion of T cell activation, T cell proliferation. NK cell activation, NK cell proliferation, or any combination thereof. In some embodiments, T cell activation and/or NK cell activation results in increased production of IFN-γ, TNF-α, perforin, or a combination thereof by T cells and/or NK cells. In some embodiments, antibodies of the present disclosure specifically bind to a CD163 protein expressed on a human macrophage, wherein the human macrophage has a first immunosuppression activity before binding the antibody and second immunosuppression activity after binding the antibody, and wherein the second immunosuppression activity lower than the first immunosuppression activity. In various embodiments, the first and second immunosuppression activities are each non-zero.

In some embodiments, an antibody of the present disclosure promotes T cell activation and proliferation. In some embodiments, the antibody skews a T cell population towards an anti-tumor T cell phenotype. In some embodiments, the antibody reduces or blocks myeloid cell suppression of T cell activation. In some embodiments, the antibody reduces the ability of TAMs to suppress T-cell activation, leading to greater T-cell stimulation and IL-2 production. In some embodiments, the antibody blocks the ability of TAMs to suppress T-cell activation, leading to greater T-cell stimulation and IL-2 production.

In some embodiments, an antibody disclosed herein reduces myeloid suppression of T cell proliferation. In some embodiments, the antibody reduces the ability of TAMs to suppress both CD4⁺ and CD8⁺ T cell activation and proliferation. In some embodiments, the antibody reduces TAM suppression of Th1 cell proliferation. Proliferated T cells show enhanced expression of activation markers on CD4⁺ T cells.

In some embodiments, an antibody of the present disclosure alters an M2-polarized macrophage such that the macrophage exhibits a M1-like phenotype that alleviates immunosuppressive effects of M2 macrophages. In some embodiments, an antibody described herein influences monocyte-derived macrophages to differentiate to a less immunosuppressive and more anti-tumor differentiation state.

In some embodiments, an antibody of the present disclosure reduces a pro-fibrotic function of a tissue-resident or infiltrating macrophages in a fibrotic tissue. In some embodiments, a reduction in a pro-fibrotic function of a tissue-resident or infiltrating macrophages in a fibrotic tissue corresponds to reduced activation and/or proliferation of fibroblasts. In some embodiments, the antibodies of the present disclosure reduce activation and/or proliferation of fibroblasts. In some embodiments, antibodies of the present disclosure specifically bind to a CD163 protein expressed on a human macrophage, such as an M2 macrophage, wherein the human macrophage has a first pro-fibrotic activity before binding the antibody and second pro-fibrotic activity after binding the antibody, and wherein the second pro-fibrotic activity is lower than the first pro-fibrotic activity. In various embodiments, the first and second pro-fibrotic activities are each non-zero. In some embodiments, the fibrosis is a primary fibrotic disease. In some embodiments, the fibrosis is a secondary fibrotic disease.

In some embodiments, an antibody of the present disclosure alters an M2-polarized macrophage such that the macrophage exhibits a M1-like phenotype that alleviates the immunosuppressive or pro-fibrotic activity of M2 macrophages. In some embodiments, an antibody described herein influences monocyte-derived macrophages to differentiate to a less pro-fibrotic and/or less immunosuppressive differentiation state. For example, an antibody described herein can disrupt the IL-10 signaling axis including disruption of IL-10 mediated polarization of macrophages towards an M2 phenotype and/or disruption of IL-10 production of M2 macrophages. In some embodiments, an antibody of the present disclosure alters an M2-polarized macrophage such that the macrophage produces and/or secretes less immunosuppressive or pro-fibrotic cytokines, including, but not limited to, TGF-β, PDGF, VEGF, IGF-1, IL-10, and Galectin-3. In some embodiments, an antibody of the present disclosure alters an M2-polarized macrophage such that the macrophage produces and/or secretes less TGF-β, PDGF, VEGF, IGF-1, Galectin-3, IL-10, or combinations thereof.

In some embodiments, provided herein are antibodies that specifically bind to huCD163 that is expressed on a human macrophage and reduces expression of at least one of CD16, CD64, TLR2, or Siglec-15 by the macrophage. In some embodiments, the human macrophage is an immunosuppressive macrophage. In some embodiments, the human macrophage is an M2-like immunosuppressive macrophage. In some embodiments, the human macrophage is a tissue-resident macrophage. In some embodiments, the tissue-resident macrophage resides in a lung, a kidney, a heart, or a liver. In some embodiments, the human macrophage is a pulmonary macrophage. In some embodiments, the human macrophage is an alveolar macrophage (AM). In some embodiments, the human macrophage is an interstitial macrophage. In some embodiments, the human macrophage is an infiltrating macrophage.

In some embodiments, an antibody disclosed herein binds to a CD163 protein that is expressed by a macrophage as a component of a complex comprising at least one other protein expressed by the macrophages. In some embodiments, the complex is a cell surface complex. In some embodiments, the complex comprises at least one other protein selected from a galectin-1 protein, a LILRB2 protein, and a casein kinase 11 protein.

In some embodiments, an antibody disclosed herein promotes CD4⁺ T cell activity or proliferation. In some embodiments, the antibody promotes expression of CD69, ICOS, OX40, PD1, LAG3, or CTLA4 by CD4⁺ T cells.

In some embodiments, an antibody disclosed herein promotes CD8⁺ T cell activity or proliferation. In some embodiments, the antibody promotes expression of ICOS, OX40, PD1, LAG3, or CTLA4 by CD8⁺ T cells.

In some embodiments, an antibody disclosed herein promotes tumor cell killing in a tumor microenvironment by promoting CD8⁺ T cell activity or proliferation. In some embodiments, the antibody promotes cytotoxic lymphocyte-mediated killing of cancer cells. In some embodiments, the antibody promotes NK cell-mediated tumor cell killing.

In some embodiments, an antibody disclosed herein promotes expression of IL-2 by T cells. In some embodiments, the binding of antibodies of the present disclosure to CD163 protein increases CD4⁺ T cells, CD196− T cells, CXCR3⁺ T cells, CCR4− T cells, or any combination thereof. In some embodiments, the antibody increases CD4⁺ CD196− CXCR3⁺ CCR4⁻ T cells.

In some embodiments, an antibody disclosed herein has a constant domain that binds to an Fc receptor expressed on a macrophage. In some embodiments, the antibody specifically binds huCD163 and has a constant domain that binds to an Fc receptor. In some embodiments, the antibody has a constant domain that binds to an Fc receptor expressed on CD163⁺ immunosuppressive myeloid cells such as CD16 (FcγRIIIa) or CD64 (FcγRI). In some embodiments, the huCD163 and Fc receptor are expressed on the same cell. In some embodiments, the huCD163 and Fc receptor are expressed on different cells. In some embodiments, the antibody variable domain specifically binds huCD163 and the antibody constant domain binding to an Fc receptor simultaneously.

In some embodiments, an antibody disclosed herein binds to a CD163 protein on a macrophage and is internalized by the macrophage.

In some embodiments, an antibody disclosed herein is not cytotoxic to a macrophage to which it is bound.

In some embodiments, an antibody disclosed herein has a constant domain that binds to an Fc receptor expressed on a macrophage. In some embodiments, the antibody specifically binds huCD163 and has a constant domain that binds to an Fc receptor. In some embodiments, the antibody has a constant domain that binds to an Fc receptor expressed on CD163⁺ immunosuppressive myeloid cells such as CD16 (FcγRIIIa) or CD64 (FcγRI). In some embodiments, the huCD163 and Fc receptor are expressed on the same cell. In some embodiments, the huCD163 and Fc receptor are expressed on different cells. In some embodiments, the antibody variable domain specifically binds huCD163 and the antibody constant domain binding to an Fc receptor simultaneously.

Disclosed herein, in certain embodiments, are antibodies that specifically bind to a CD163 protein expressed on human M2 and M2-like macrophages, wherein said binding results in at least one of the following effects:
(a) reduced expression of at least one marker by the human macrophage, wherein the at least one marker is CD16, CD64, TLR2, or Siglec-15;
(b) internalization of the antibody by the human macrophage;
(c) reduces activation and/or proliferation of fibroblasts; and
(d) reduces secretion by the macrophage of TGF-β, PDGF, VEGF, IGF-1, Galectin-3, IL-10, or combinations thereof.

In some embodiments, an antibody disclosed herein selectively binds to human CD163⁺ immunosuppressive myeloid cells in a tissue-resident macrophage population, in which the antibody specifically binds to a CD163 protein expressed on the M2 macrophages of the tissue-resident population. In some embodiments, an antibody disclosed herein selectively binds to human CD163⁺ immunosuppressive myeloid cells in an infiltrating macrophage population, in which the antibody specifically binds to a CD163 protein expressed on the M2 macrophages and reduces an immunosuppressive activity of the infiltrating population.

In some embodiments, an antibody disclosed herein selectively binds to human CD163⁺ immunosuppressive myeloid cells in a tumor microenvironment or fibrotic tissue, in which the antibody specifically binds to a CD163 protein expressed on the M2 macrophages and reduces a M2 macrophage-mediated pro-fibrotic function or immunosuppression. In some embodiments, an antibody disclosed herein is human, humanized, or chimeric. In some embodiments, an antibody disclosed herein is an antigen-binding fragments thereof that bind as described.

In some embodiments the antibodies of the present disclosure are intact immunoglobulin molecules, such as, for example, a human antibody, as well as those portions of a humanized Ig molecule that contain the antigen-binding site (i.e., paratope) or a single heavy chain and a single light chain, including those portions known in the art as Fab, F(ab′), F(ab′)₂, Fd, scFv, a variable heavy domain, a variable light domain, a variable NAR domain, bi-specific scFv, a bi-specific Fab₂, a tri-specific Fab₃ a single chain binding polypeptide, a dAb fragment, a diabody, and others also referred to as antigen-binding fragments. When constructing an immunoglobulin molecule or fragments thereof, variable regions or portions thereof are, in some embodiments, fused to, connected to, or otherwise joined to one or more constant regions or portions thereof to produce any of the antibodies or fragments thereof described herein. Thus, in some embodiments, the antigen-binding fragment of any one of the antibodies described above is a Fab, a F(ab′), a Fd, a F(ab′)₂, a Fv, a scFv, a single chain binding polypeptide (e.g., a scFv with Fc portion) or any other functional fragment thereof as described herein.

In some embodiments, antibodies of the present disclosure are of any immunoglobulin class, and, therefore, in some embodiments, have a gamma, mu, alpha, delta, or epsilon heavy chain. In some embodiments, the gamma chain is gamma 1, gamma 2, gamma 3, or gamma 4. In some embodiments, the alpha chain is alpha 1 or alpha 2.

In some embodiments, an antibody of the present disclosure is an IgG immunoglobulin. In some embodiments, antibodies of the present disclosure are of any IgG subclass. In some embodiments the antibody is IgG1.

In some embodiments, antibodies of the present disclosure comprise a variable light chain that is either kappa or lambda. In some embodiments, the lambda chain is of any of subtype, including, e.g., lambda 1, lambda 2, lambda 3, and lambda 4. In some embodiments, the light chain is kappa.

In some embodiments, antibodies disclosed herein comprise a human variable framework region and a human constant region. In some embodiments the antibodies comprise a human light chain variable framework region and a human light chain constant region. In some embodiments the antibodies comprise a human heavy chain variable framework region and a human heavy chain constant region. In some embodiments the antibodies comprise a human light chain variable framework region, a human light chain constant region, a human heavy chain variable framework region, and a human heavy chain constant region.

In some embodiments, an antibody of the present disclosure comprises a human variable framework region and a murine constant region. In some embodiments, an antibody of the present disclosure comprises a human heavy chain variable framework region and a murine heavy chain constant region. In some embodiments, an antibody of the present disclosure comprises a human light chain variable framework region, a murine light chain constant region, a human heavy chain variable framework region, and a murine heavy chain constant region.

Binding of an antibody or antigen-binding fragment to a CD163 protein expressed on M2 macrophages are partially (e.g., 5%, 10%, 20%, 30%, 40%, 50%, 60%, 70%, 80%, 90%, 95%, 98%, 99% or any number therein) or completely modulate a biological function of such M2 macrophages in some embodiments. The activity of an antibody or antigen-binding fragment, for example, are determined using an in vitro assay and/or in vivo using art-recognized assays such as those described herein or otherwise known in the art.

In some embodiments, antibodies of the present disclosure are further modified to alter the specific properties of the antibody while retaining the desired functionality, if needed. For example, in one embodiment, an antibody of the present disclosure is modified to alter a pharmacokinetic property of the antibody, including, but not limited to, in vivo stability, solubility, bioavailability, or half-life.

In some embodiments, an antibody described herein has a dissociation constant (K_{D}) of about 1 to about 10 pM, from about 10 to about 20 pM, from about 1 to about 29 pM, from about 30 to about 40 pM, from about 10 to about 100 pM, or from about 20 to about 500 pM.

In some embodiments, an antibody described herein has a dissociation constant (K_{D}) of less than about 500 pM, less than about 400 pM, less than about 300 pM, less than about 200 pM, less than about 100 pM, less than about 75 pM, less than about 50 pM, less than about 30 pM, less than about 25 pM, less than about 20 pM, less than about 18 pM, less than about 15 pM, less than about 10 pM, less than about 75. pM, less than about 5 pM, less than about 2.5 pM, or less than about 1 pM.

In some embodiments, an antibody described herein has an affinity for a huCD163 protein or peptide of from about 10⁻⁹ to about 10⁻¹⁴, from about 10⁻¹⁰ to about 10⁻¹⁴, from about 10⁻¹¹ to about 10⁻¹⁴, from about 10⁻¹² to about 10⁻¹, from about 10⁻¹³ to about 10⁻¹⁴, from about 10⁻¹⁰ to about 10⁻¹¹, from about 10⁻¹¹ to about 10⁻¹², from about 10⁻¹² to about 10⁻¹³, or 10⁻¹³ to about 10⁻¹⁴ M.

In some embodiments, an antibody described herein has more than one binding site. In some embodiments, the binding sites are identical to one another. In some embodiments, the binding sites are different from one another. A naturally occurring human immunoglobulin typically has two identical binding sites, while engineered antibodies, for example, have two or more different binding sites.

In some embodiments, an antibody of the present disclosure is bispecific or multispecific. Bispecific antibodies are antibodies that have binding specificities for at least two different epitopes. Exemplary bispecific antibodies, in some embodiments, bind to two different epitopes of a single antigen. Other such antibodies, in some embodiments, combine a first antigen binding site with a binding site for a second antigen. In some embodiments, the bispecific antibodies bind at least two different epitopes and have constant domains that bind to Fc receptors. In some embodiments, the binding of one or more epitopes of the bispecific antibodies is simultaneous with binding of the constant domains of the bispecific antibodies to Fc receptors.

In some embodiments, an antibody of the present disclosure has two or more valencies, which are also referred to as multivalent. In some embodiments, an antibody of the present disclosure is trispecific. In some embodiments, the multivalent antibody is internalized (and/or catabolized) faster than a bivalent antibody by a cell expressing an antigen to which the antibodies bind. In some embodiments, the antibodies of the present disclosure are multivalent antibodies with three or more antigen binding sites (e.g., tetravalent antibodies). In some embodiments, the multivalent antibodies of the present disclosure are produced by recombinant expression of nucleic acid encoding the polypeptide chains of the antibody. In some embodiments, the multivalent antibody comprises a dimerization domain and three or more antigen binding sites. In some embodiments, the dimerization domain comprises (or consists of) an Fc region or a hinge region. In this scenario, the antibody will comprise an Fc region and three or more antigen binding sites amino-terminal to the Fc region. In some embodiments, the multivalent antibody herein comprises about three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) comprises VD1-(X1)ₙ-VD2-(X2)ₙ-Fc, wherein VD1 is a first variable region, VD2 is a second variable region, Fc is one polypeptide chain of an Fc region. X1 and X2 represent an amino acid or polypeptide, and n is 0 or 1. In some embodiments, the polypeptide chain(s) each independently comprise: V_{H}—C_{H}1-flexible linker-V_{H}-C_{H}1-Fc region chain; or V_{H}-C_{H}1-V_{H}-C_{H}1-Fc region chain. In some embodiments, the multivalent antibody herein further comprises at least two (and preferably four) light chain variable region polypeptides. In some embodiments, the multivalent antibody herein comprises from about two to about eight light chain variable region polypeptides. In some embodiments, the light chain variable region polypeptides described herein comprise a light chain variable region. In some embodiments, the light chain variable region polypeptides described herein further comprise a C_{L} domain.

In some embodiments, an antibody of the present disclosure is constructed to fold into multivalent forms, which, in some embodiments, improves binding affinity, specificity, and/or increased half-life in blood. Multivalent forms of antibodies are prepared, for example, by techniques known in the art.

In some embodiments, an antibody of the present disclosure is an SMIP or binding domain immunoglobulin fusion protein specific for the target protein. These constructs are single-chain polypeptides comprising antigen-binding domains fused to immunoglobulin domains necessary to carry out antibody effector functions.

In some embodiments, an antibody of the present disclosure comprises a single chain binding polypeptide having a heavy chain variable region, and/or a light chain variable region which binds an epitope disclosed herein and has, optionally, an immunoglobulin Fc region. Such a molecule is a single chain variable fragment (scFv) optionally having effector function or increased half-life through the presence of the immunoglobulin Fc region.

### Anti-CD163 Antibodies

Provided herein, in certain embodiments, are antibodies that specifically bind to CD163 proteins. In some embodiments, CD163-binding antibodies comprise at least one heavy chain and at least one light chain. In some embodiments, CD163-binding antibodies comprise at least one heavy chain comprising a heavy chain variable domain (V_{H}) and at least one light chain comprising a light chain variable domain (V_{L}). Each V_{H} and V_{L} comprises three complementarity determining regions (CDR). The amino acid sequences of the V_{H} and V_{L} and the CDRs determine the antigen binding specificity and antigen binding strength of the antibody. The V_{H} and V_{L} domains are summarized in Table 1. The amino acid sequences of the CDRs are summarized in Table 2 and Table 3.

In some embodiments, an antibody disclosed herein is a monoclonal antibody. In some embodiments, an antibody disclosed herein is an antigen binding fragment. In some embodiments, an antibody disclosed herein is selected from a whole immunoglobulin, an scFv, a Fab, a F(ab′)₂, or a disulfide linked Fv. In some embodiments, an antibody disclosed herein is an IgG or an IgM. In some embodiments, an antibody disclosed herein is humanized. In some embodiments, an antibody disclosed herein is chimeric.

### Anti-CD163 Antibody Variable Domains

TABLE 1
Anti-CD163 Variable Domain Sequences.

| | | SEQ |
|---|---|---|
| | SEQUENCE | ID NO: |
| V1 Light Chain | DIQMTQSPSSLSASVGDRVTITCRASQSISRYLNWYQQKPGKA | 28 |
| Variable | PKLLIYAASSLQSGVPSRFSGSGSGTDFTLTISSLQPEDFATY | |
| domain | YCQQSYSTQRGSFGQGTKVEIKR | |
| V1 Heavy | EVQLVESGGGVVQPGRSLRLSCAASGFTFSSYDMHWVRQAPGK | 29 |
| Chain Variable | GLEWVAVISEDGSNKYNADSVKGRFTISRDNSKNTLYLQMNSL | |
| domain | RAEDTAVYYCARENVRPYYDFWSGYSSEYYYYGLDVWGQGTTV | |
| | TVS | |
| V2 Light Chain | DIQMTQSPSSLSASVGDRVTITCRASQSISRYLNWYQQKPGKA | 30 |
| Variable | PKLLIYAASSLQNGVPSRFSGSGSGTDFTLTISSLQPEDFATY | |
| domain | YCQQSYSTTRGSFGQGTKVEIKR | |
| V2 Heavy | EVQLVESGGGVVQPGRSLRLSCAASGFTFSSETMHWVRQAPGK | 31 |
| Chain Variable | GLEWVAVISEDGSNKYHADSVKGRFTISRDNSKNTLYLQMNSL | |
| domain | RAEDTAVYYCARENVRPYYDFWSGYNSEYYYYGMDVWGQGTTV | |
| | TVSS | |
| V3 Light Chain | DIQMTQSPSSLSASVGDRVTITCRASQSISRYLNWYQQKPGKA | 32 |
| Variable | PKLLIYAASSLQSGVPSRFSGSGSGTDFTLTISSLQPEDFATY | |
| domain | YCQQSYSTQRGAFGQGTKVEIKR | |
| V3 Heavy | EVQLVESGGGVVQPGRSLRLSCAASGFTFSSYVMHWVRQAPGK | 33 |
| Chain Variable | GLEWVAVISEDGSNKYEADSVKGRFTISRDNSKNTLYLQMNSL | |
| domain | RAEDTAVYYCARENVRPYYDFWRGYNSEYYYYGLDVWGQGTTV | |
| | TVSS | |
| V4 Light Chain | DIQMTQSPSSLSASVGDRVTITCRASQSISRYLNWYQQKPGKA | 34 |
| Variable | PKLLIYAASSLQSGVPSRFSGSGSGTDFTLTISSLQPEDFATY | |
| domain | YCQQSYSTQRGSFGQGTKVEIKR | |
| V4 Heavy | EVQLVESGGGVVQPGRSLRLSCAASGFTFSSYVMHWVRQAPGK | 35 |
| Chain Variable | GLEWVAVISEDGSNKYNADSVKGRFTISRDNSKNTLYLQMNSL | |
| domain | RAEDTAVYYCARENVRPYYDFWSGYSSEYYYYGLDVWGQGTTV | |
| | TVSS | |
| V5 Light Chain | DIQMTQSPSSLSASVGDRVTITCRASQSISRYLNWYQQKPGKA | 36 |
| Variable | PKLLIYAASSLQSGVPSRFSGSGSGTDFTLTISSLQPEDFATY | |
| domain | YCQQSYSTTRGSFGQGTKVEIKR | |
| V5 Heavy | EVQLVESGGGVVQPGRSLRLSCAASGFTFSSYDMHWVRQAPGK | 37 |
| Chain Variable | GLEWVAVISEDGSNKYNADSVKGRFTISRDNSKNTLYLQMNSL | |
| domain | RAEDTAVYYCARENVRPYYDFWRGYNSEYYYYGLDVWGQGTTV | |
| | TVSS | |
| V6 Light Chain | DIQMTQSPSSLSASVGDRVTITCRASQSISRYLNWYQQKPGKA | 38 |
| Variable | PKLLIYAASSLQSGVPSRFSGSGSGTDFTLTISSLQPEDFATY | |
| domain | YCQQSYSTTGGTFGQGTKVEIKR | |
| V6 Heavy | EVQLVESGGGVVQPGRSLRLSCAASGFTFSSETMHWVRQAPGK | 39 |
| Chain Variable | GLEWVAVISEDGSNKYNADSVKGRFTISRDNSKNTLYLQMNSL | |
| domain | RAEDTAVYYCARENVRPYYDFWSGYSSEYYYYGLDVWGQGTTV | |
| | TVSS | |
| AB101 Light | DIQMTQSPSSLSASVGDRVTITCRASQSISSYLNWYQQKPGKA | 40 |
| Chain Variable | PKLLIYAASSLQSGVPSRFSGSGSGTDFTLTISSLQPEDFATY | |
| domain | YCQQSYSTPRGTFGQGTKVEIKR | |
| AB101 Heavy | EVQLVESGGGVVQPGRSLRLSCAASGFTFSSYAMHWVRQAPGK | 41 |
| Chain Variable | GLEWVAVISYDGSNKYYADSVKGRFTISRDNSKNTLYLQMNSL | |
| domain | RAEDTAVYYCARENVRPYYDFWSGYYSEYYYYGMDVWGQGTTV | |
| | TVSSASTKGPSVFPLAPSSKSTSGGTAALGCLVKDYF | |

*Underlined text in Table 1 indicates CDRs, with domain boundary annotations based on the IMGT database and the CDR region annotations based on the Honegger (AHo) numbering scheme.

Disclosed herein, in certain embodiments, are antibodies comprising a light chain variable domain (V_{L}) having an amino acid sequence at least about 70% identical to an amino acid sequence set forth as SEQ ID NO: 28. In some embodiments, the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 28. In some embodiments, the V_{L} has an amino acid sequence I100% identical to an amino acid sequence set forth as SEQ ID NO: 28.

Disclosed herein, in certain embodiments, are antibodies comprising a light chain variable domain (V_{L}) having an amino acid sequence at least about 70% identical to an amino acid sequence set forth as SEQ ID NO: 30. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 30. In some embodiments, the V_{L} has an amino acid sequence I100% identical to an amino acid sequence set forth as SEQ ID NO: 30.

Disclosed herein, in certain embodiments, are antibodies comprising a light chain variable domain (V_{L}) having an amino acid sequence at least about 70% identical to an amino acid sequence set forth as SEQ ID NO: 32. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 32. In some embodiments, the V_{L} has an amino acid sequence 100% identical to an amino acid sequence set forth as SEQ ID NO: 32.

Disclosed herein, in certain embodiments, are antibodies comprising a light chain variable domain (V_{L}) having an amino acid sequence at least about 70% identical to an amino acid sequence set forth as SEQ ID NO: 34. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 34. In some embodiments, the V_{L} has an amino acid sequence 100% identical to an amino acid sequence set forth as SEQ ID NO: 34.

Disclosed herein, in certain embodiments, are antibodies comprising a light chain variable domain (V_{L}) having an amino acid sequence at least about 70% identical to an amino acid sequence set forth as SEQ ID NO: 36. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 36. In some embodiments, the V_{L} has an amino acid sequence 100% identical to an amino acid sequence set forth as SEQ ID NO: 36.

Disclosed herein, in certain embodiments, are antibodies comprising a light chain variable domain (V_{L}) having an amino acid sequence at least about 70% identical to an amino acid sequence set forth as SEQ ID NO: 38. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 38. In some embodiments, the VL has an amino acid sequence 100% identical to an amino acid sequence set forth as SEQ ID NO: 38.

Disclosed herein, in certain embodiments, are antibodies comprising a heavy chain variable domain (V_{H}) having an amino acid sequence at least about 70% identical to an amino acid sequence set forth as SEQ ID NO: 29. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 29. In some embodiments, the V_{H} has an amino acid sequence 100% identical to an amino acid sequence set forth as SEQ ID NO: 29.

Disclosed herein, in certain embodiments, are antibodies comprising a heavy chain variable domain (V_{H}) having an amino acid sequence at least about 70% identical to an amino acid sequence set forth as SEQ ID NO: 31. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 31. In some embodiments, the V_{H} has an amino acid sequence 100% identical to an amino acid sequence set forth as SEQ ID NO: 31.

Disclosed herein, in certain embodiments, are antibodies comprising a heavy chain variable domain (V_{H}) having an amino acid sequence at least about 70% identical to an amino acid sequence set forth as SEQ ID NO: 33. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 33. In some embodiments, the V_{H} has an amino acid sequence 100% identical to an amino acid sequence set forth as SEQ ID NO: 33.

Disclosed herein, in certain embodiments, are antibodies comprising a heavy chain variable domain (V_{H}) having an amino acid sequence at least about 70% identical to an amino acid sequence set forth as SEQ ID NO: 35. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 35. In some embodiments, the V_{H} has an amino acid sequence 100% identical to an amino acid sequence set forth as SEQ ID NO: 35.

Disclosed herein, in certain embodiments, are antibodies comprising a heavy chain variable domain (V_{H}) having an amino acid sequence at least about 70% identical to an amino acid sequence set forth as SEQ ID NO: 37. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 37. In some embodiments, the V_{H} has an amino acid sequence 100% identical to an amino acid sequence set forth as SEQ ID NO: 37.

Disclosed herein, in certain embodiments, are antibodies comprising a heavy chain variable domain (V_{H}) having an amino acid sequence at least about 70% identical to an amino acid sequence set forth as SEQ ID NO: 39. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 39. In some embodiments, the V_{H} has an amino acid sequence 100% identical to an amino acid sequence set forth as SEQ ID NO: 39.

Disclosed herein, in certain embodiments, are antibodies comprising a light chain variable domain (V_{L}) having an amino acid sequence at least about at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence set forth in the group consisting of: SEQ ID NO: 28, SEQ ID NO: 30, SEQ ID NO: 32, SEQ ID NO: 34, SEQ ID NO: 36, and SEQ ID NO: 38; and a heavy chain variable domain (V_{H}) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence set forth as in the group consisting of: SEQ ID NO: 29, SEQ ID NO: 31, SEQ ID NO: 33, SEQ ID NO: 35, SEQ ID NO: 37, and SEQ ID NO: 39, provided that the antibody does not comprise the light chain variable sequence of SEQ ID NO: 40 and heavy chain variable sequence of SEQ ID NO: 41. In some embodiments, the sequence of the antibody is 100% identical at CDR H1, CDR H2, CDR H2, CDR L1, CDR L2, and CDR L3.

Disclosed herein, in certain embodiments, are antibodies comprising a light chain variable domain (V_{L}) having an amino acid sequence at least about at least about 80% identical to an amino acid sequence set forth in the group consisting of: SEQ ID NO: 28, SEQ ID NO: 30, SEQ ID NO: 32, SEQ ID NO: 34, SEQ ID NO: 36, and SEQ ID NO: 38, and a heavy chain variable domain (V_{H}) having an amino acid sequence at least about 80% identical to an amino acid sequence set forth as in the group consisting of: SEQ ID NO: 29, SEQ ID NO: 31, SEQ ID NO: 33, SEQ ID NO: 35, SEQ ID NO: 37, and SEQ ID NO: 39; provided that the antibody does not comprise the light chain variable sequence of SEQ ID NO: 40 and the heavy chain variable sequence of SEQ ID NO: 41. In some embodiments, the sequence of the antibody is 100% identical at CDR H1, CDR H2, CDR H2, CDR L1, CDR L2, and CDR L3.

### Exemplary Anti-CD163 Complementarity Determining Regions

TABLE 2
anti-CD163 Light Chain CDR Sequences

| | CDR L1 | CDR L2 | CDR L3 |
|---|---|---|---|
| AB101 | RASQSISSYLN | AASSLQS | QQSYSTPRGT |
| | (SEQ ID NO: 1) | (SEQ ID NO: 2) | (SEQ ID NO: 3) |
| V1 | RASQSISRYLN | AASSLQS | QQSYSTQRGS |
| | (SEQ ID NO: 7) | (SEQ ID NO: 2) | (SEQ ID NO: 8) |
| V2 | RASQSISRYLN | AASSLQN | QQSYSTTRGS |
| | (SEQ ID NO: 7) | (SEQ ID NO: 9) | (SEQ ID NO: 10) |
| V3 | RASQSISRYLN | AASSLQS | QQSYSTQRGA |
| | (SEQ ID NO: 7) | (SEQ ID NO: 2) | (SEQ ID NO: 11) |
| V4 | RASQSISRYLN | AASSLQS | QQSYSTQRGS |
| | (SEQ ID NO: 7) | (SEQ ID NO: 2) | (SEQ ID NO: 8) |
| V5 | RASQSISRYLN | AASSLQS | QQSYSTTRGS |
| | (SEQ ID NO: 7) | (SEQ ID NO: 2) | (SEQ ID NO: 10) |
| V6 | RASQSISRYLN | AASSLQS | QQSYSTTGGT |
| | (SEQ ID NO: 7) | (SEQ ID NO: 2) | (SEQ ID NO: 12) |
| | RASQSISX₈YLN | AASSLQX₉ | QQSYSTX₁₀X₁₁GX₁₂ |
| | (SEQ ID NO: 13) | (SEQ ID NO: 14) | (SEQ ID NO: 15) |
| | X₈ = S, R, K, H | X₉ = S, N, Q, T | X₁₀ = P, Q, T, |
| | | | S, N, A, G |
| | | | X₁₁ = R, G, A, |
| | | | S |
| | | | X₁₂ = T, S, A, |
| | | | G, N |

TABLE 3
anti-CD163 Heavy Chain CDR Sequences

| | CDR H1 | CDR H2 | CDR H3 |
|---|---|---|---|
| AB101 | SYAMH | VISYDGSNKYYADSV | ENVRPYYDFWSGYYS |
| | (SEQ ID | KG | EYYYYGMDV |
| | NO: 4) | (SEQ ID NO: 5) | (SEQ ID NO: 6) |
| V1 | SYDMH | VISEDGSNKYNADSV | ENVRPYYDFWSGYSS |
| | (SEQ ID | KG | EYYYYGLDV |
| | NO: 16) | (SEQ ID NO: 17) | (SEQ ID NO: 18) |
| V2 | SETMH | VISEDGSNKYHADSV | ENVRPYYDFWSGYNS |
| | (SEQ ID | KG | EYYYYGMDV |
| | NO: 19) | (SEQ ID NO: 20) | (SEQ ID NO: 21) |
| V3 | SYVMH | VISEDGSNKYEADSV | ENVRPYYDFWRGYNS |
| | (SEQ ID | KG | EYYYYGLDV |
| | NO: 22) | (SEQ ID NO: 23) | (SEQ ID NO: 24) |
| V4 | SYVMH | VISEDGSNKYNADSV | ENVRPYYDFWSGYSS |
| | (SEQ ID | KG | EYYYYGLDV |
| | NO: 22) | (SEQ ID NO: 17) | (SEQ ID NO: 18) |
| V5 | SYDMH | VISEDGSNKYNADSV | ENVRPYYDFWRGYNS |
| | (SEQ ID | KG | EYYYYGLDV |
| | NO: 16) | (SEQ ID NO: 17) | (SEQ ID NO: 24) |
| V6 | SETMH | VISEDGSNKYNADSV | ENVRPYYDFWSGYSS |
| | (SEQ ID | KG | EYYYYGLDV |
| | NO: 19) | (SEQ ID NO: 17) | (SEQ ID NO: 18) |
| | SX₁X₂MH | VISX₃DGSNKYX₄ADS | ENVRPYYDFWX₅GYX₆ |
| | (SEQ ID | VKG | SEYYYYGX₇DV |
| | NO: 25) | (SEQ ID NO: 26) | (SEQ ID NO: 27) |
| | X₁ = Y, E, | X₃ = Y, E, Q, D | X₅ = S, R, K, H |
| | Q, D | X₄ = Y, N, H, E, | X₆ = Y, S, N, T, |
| | X₂ = A, D, | D, K, Q, R | A, Q |
| | T, V, S, | | X₇ = M, L, I, V |
| | G, E | | |

Disclosed herein, in certain embodiments, are antibodies binding to CD163 comprising a light chain CDR1 (CDR L1) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 (CDR L2) having an amino acid sequence at least about 70%, 75%, 80%, 81%, 82%, 83%, 84%, 85%, 86%, 87%, 88%, 89%. 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 (CDR L3) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; provided that the antibody does not comprise a sequence as set forth in SEQ ID NO: 1, SEQ ID NO: 2, and SEQ ID NO: 3.

In some embodiments, antibodies binding to CD163 comprise a light chain CDR1 having an amino acid sequence 100% identical to an amino acid sequence set forth in the group consisting of: SEQ ID NO: 1, SEQ ID NO: 7, and SEQ ID NO: 13; a light chain CDR2 having an amino acid sequence 100% identical to an amino acid sequence set forth in the group consisting of: SEQ ID NO: 2, SEQ ID NO: 9, and SEQ ID NO: 14; and a light chain CDR3 having an amino acid sequence 100% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; provided that the antibody does not comprise a sequence as set forth in SEQ ID NO: 1, SEQ ID NO: 2, and SEQ ID NO: 3.

Disclosed herein, in certain embodiments, are antibodies binding to CD163 comprising a heavy chain CDR1 (CDR H1) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 (CDR H2) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 (CDR H3) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6. SEQ ID NO: 18, SEQ ID NO: 21, SEQ ID NO: 24, and SEQ ID NO: 27; provided that the antibody does not comprise a sequence as set forth in SEQ ID NO: 4, SEQ ID NO: 5, and SEQ ID NO: 6.

In some embodiments, antibodies binding to CD163 comprise a heavy chain CDR1 having an amino acid sequence 100% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence 100% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence 100% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence at least about 100% identical to a sequence as set forth in SEQ ID NO: 4, SEQ ID NO: 5, and SEQ ID NO: 6.

Disclosed herein, in certain embodiments, are antibodies comprising (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 8(0%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 1, SEQ ID NO: 2, SEQ ID NO: 3, SEQ ID NO: 4, SEQ ID NO: 5, and SEQ ID NO: 6.

In some embodiments, antibodies binding to CD163 comprise (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence s set forth in SEQ ID NO: 1, SEQ ID NO: 2. SEQ ID NO: 3, SEQ ID NO: 4, SEQ ID NO: 5, and SEQ ID NO: 6.

### Exemplary Antibodies

In some embodiments, the antibody is V1 and comprises a light chain variable domain (V_{L}) having an amino acid sequence as set forth as SEQ ID NO: 28, and a heavy chain variable domain (V_{H}) having an amino acid sequence as set forth as SEQ ID NO: 29.

In some embodiments, the antibody is V2 and comprises a light chain variable domain (V_{L}) having an amino acid sequence as set forth as SEQ ID NO: 30, and a heavy chain variable domain (V_{H}) having an amino acid sequence as set forth as SEQ ID NO: 31.

In some embodiments, the antibody is V3 and comprises a light chain variable domain (V_{L}) having an amino acid sequence as set forth as SEQ ID NO: 32, and a heavy chain variable domain (V_{H}) having an amino acid sequence as set forth as SEQ ID NO: 33.

In some embodiments, the antibody is V4 and comprises a light chain variable domain (V_{L}) having an amino acid sequence as set forth as SEQ ID NO: 34, and a heavy chain variable domain (V_{H}) having an amino acid sequence as set forth as SEQ ID NO: 35.

In some embodiments, the antibody is V5 and comprises a light chain variable domain (V_{L}) having an amino acid sequence as set forth as SEQ ID NO: 36, and a heavy chain variable domain (V_{H}) having an amino acid sequence as set forth as SEQ ID NO: 37.

In some embodiments, the antibody is V6 and comprises a light chain variable domain (V_{L}) having an amino acid sequence as set forth as SEQ ID NO: 38, and a heavy chain variable domain (V_{H}) having an amino acid sequence as set forth as SEQ ID NO: 39.

In some embodiments, the antibody is V1 and comprises a CDR L1 having an amino acid sequence as set forth as SEQ ID NO: 7. CDR L2 having an amino acid sequence as set forth as SEQ ID NO: 2, and CDR L3 having an amino acid sequence as set forth as SEQ ID NO: 8; and a CDR H1 having an amino acid sequence as set forth as SEQ ID NO: 16, CDR H2 having an amino acid sequence as set forth as SEQ ID NO: 17, and CDR H3 having an amino acid sequence as set forth as SEQ ID NO: 18.

In some embodiments, the antibody is V2 and comprises a CDR L1 having an amino acid sequence as set forth as SEQ ID NO: 7, CDR L2 having an amino acid sequence as set forth as SEQ ID NO: 9, and CDR L3 having an amino acid sequence as set forth as SEQ ID NO: 10; and a CDR H1 having an amino acid sequence as set forth as SEQ ID NO: 19, CDR H2 having an amino acid sequence as set forth as SEQ ID NO: 20, and CDR H3 having an amino acid sequence as set forth as SEQ ID NO: 21.

In some embodiments, the antibody is V3 and comprises a CDR L1 having an amino acid sequence as set forth as SEQ ID NO: 7, CDR L2 having an amino acid sequence as set forth as SEQ ID NO: 2, and CDR L3 having an amino acid sequence as set forth as SEQ ID NO: 11; and a CDR H1 having an amino acid sequence as set forth as SEQ ID NO: 22, CDR H2 having an amino acid sequence as set forth as SEQ ID NO: 23, and CDR H3 having an amino acid sequence as set forth as SEQ ID NO: 24.

In some embodiments, the antibody is V4 and comprises a CDR L1 having an amino acid sequence as set forth as SEQ ID NO: 7, CDR L2 having an amino acid sequence as set forth as SEQ ID NO: 2, and CDR L3 having an amino acid sequence as set forth as SEQ ID NO: 8; and a CDR H1 having an amino acid sequence as set forth as SEQ ID NO: 22, CDR H2 having an amino acid sequence as set forth as SEQ ID NO: 17, and CDR H3 having an amino acid sequence as set forth as SEQ ID NO: 18.

In some embodiments, the antibody is V5 and comprises a CDR L1 having an amino acid sequence as set forth as SEQ ID NO: 7, CDR L2 having an amino acid sequence as set forth as SEQ ID NO: 2, and CDR L3 having an amino acid sequence as set forth as SEQ ID NO: 10; and a CDR H1 having an amino acid sequence as set forth as SEQ ID NO: 16. CDR H2 having an amino acid sequence as set forth as SEQ ID NO: 17, and CDR H3 having an amino acid sequence as set forth as SEQ ID NO: 24.

In some embodiments, the antibody is V6 and comprises a CDR L1 having an amino acid sequence as set forth as SEQ ID NO: 7, CDR L2 having an amino acid sequence as set forth as SEQ ID NO: 2, and CDR L3 having an amino acid sequence as set forth as SEQ ID NO: 12; and a CDR H1 having an amino acid sequence as set forth as SEQ ID NO: 19, CDR H2 having an amino acid sequence as set forth as SEQ ID NO: 17, and CDR H3 having an amino acid sequence as set forth as SEQ ID NO: 18.

In some embodiments, the antibody comprises a CDR L1 having an amino acid sequence as set forth as SEQ ID NO: 13, CDR L2 having an amino acid sequence as set forth as SEQ ID NO: 14, and CDR L3 having an amino acid sequence as set forth as SEQ ID NO: 15, and a CDR H1 having an amino acid sequence as set forth as SEQ ID NO: 25, CDR H2 having an amino acid sequence as set forth as SEQ ID NO: 26, and CDR H3 having an amino acid sequence as set forth as SEQ ID NO: 27; provided that the antibody does not comprise a CDR L1 having an amino acid sequence as set forth as SEQ ID NO: 1, CDR L2 having an amino acid sequence as set forth as SEQ ID NO: 2, a CDR L3 having an amino acid sequence as set forth as SEQ ID NO: 3, CDR H1 having an amino acid sequence as set forth as SEQ ID NO: 4, CDR H2 having an amino acid sequence as set forth as SEQ ID NO: 5, and CDR H3 having an amino acid sequence as set forth as SEQ ID NO: 6.

### Binding Affinity and Immunoreactivity

Binding affinity and/or avidity of antibodies or antigen-binding fragments thereof are improved by modifying framework regions. Any suitable methods for modifications of framework regions are known in the art and are contemplated herein. Selection of one or more relevant framework amino acid positions to alter depends on a variety of criteria. One criterion for selecting relevant framework amino acids to change is, for example, the relative differences in amino acid framework residues between the donor and acceptor molecules. Selection of relevant framework positions to alter using this approach has the advantage of avoiding any subjective bias in residue determination or any bias in CDR binding affinity contribution by the residue.

Binding interactions are manifested as an intermolecular contact with one or more amino acid residues of one or more CDRs in some embodiments. Antigen-binding involves, for example, a CDR or a CDR pair or, in some cases, interactions of up to all six CDRs of the V_{H} and V_{L} chains.

Binding affinity and avidity of antibodies or antigen-binding fragments can be measured by surface plasmon resonance (SPR) measurements, AlphaLisa assays or flow cytometry of the equilibrium dissociation constant (K_{D}).

Disclosed herein are antibodies that specifically bind to human CD163 with a K_{D} from 0.1 nM to 1000 nM. In some embodiments, the antibodies specifically bind to human CD163 with a K_{D} from about 0.1 to about 500 nM, from about 0.1 to about 100 nM, from about 0.1 to about 50 nM, from about 0.1 to about 20 nM, from about 0.1 to about 10 nM, from about 0.1 to about 5 nM, from about 0.1 to about 2 nM, from about 0.1 to about 1 nM, from about 0.1 to about 0.5 nM, from about 0.5 to about 1000 nM, from about 0.5 to about 500 nM, from about 0.5 to about 100 nM, from about 0.5 to about 50 nM, from about 0.5 to about 20 nM, from about 0.5 to about 10 nM, from about 0.5 to about 5 nM, from about 0.5 to about 2 nM, from about 0.5 to about 1 nM, from about 1 to about 1000 nM, from about 1 to about 500 nM, from about 1 to about 100 nM, from about 1 to about 50 nM, from about 1 to about 20 nM, from about 1 to about 10 nM, from about 1 to about 5 nM, from about 1 to about 2 nM, from about 2 to about 1000 nM, from about 2 to about 500 nM, from about 2 to about 100 nM, from about 2 to about 50 nM, from about 2 to about 20 nM, from about 2 to about 10 nM, from about 2 to about 5 nM, from about 5 to about 1000 nM, from about 5 to about 500 nM, from about 5 to about 100 nM, from about 5 to about 50 nM, from about 5 to about 20 nM, from about 5 to about 10 nM, from about 10 to about 1000 nM, from about 10 to about 500 nM, from about 10 to about 100 nM, from about 10 to about 50 nM, from about 10 to about 20 nM, from about 20 to about 1000 nM, from about 20 to about 500 nM, from about 20 to about 100 nM, from about 20 to about 50 nM, from about 50 to about 1000 nM, from about 50 to about 500 nM, from about 50 to about 100 nM, from about 100 to about 500 nM, from about 100 to about 1000 nM, from about 500 to about 1000 nM. In some embodiments, the antibodies specifically bind to human CD163 with a K_{D} of 1.8 nM, 12 nM, 45 nM or 89 nM.

In some embodiments, an antibody disclosed herein specifically bind to human CD163 with a K_{D} of 0.824 nM. In some embodiments, an antibody disclosed herein specifically bind to human CD163 with a K_{D} of 0.937 nM. In some embodiments, an antibody disclosed herein specifically bind to human CD163 with a K_{D} of 0.964 nM. In some embodiments, an antibody disclosed herein specifically bind to human CD163 with a K_{D} of 0.991 nM. In some embodiments, an antibody disclosed herein specifically bind to human CD163 with a K_{D} of 1.03 nM. In some embodiments, an antibody disclosed herein specifically bind to human CD163 with a K_{D} of 1.25 nM.

The antibodies disclosed herein binds to the myeloid scavenger receptor CD163, which is highly expressed on M2 macrophages. The binding affinity between the antibodies disclosed herein and IL-10 polarized M2c macrophages are measured by flow cytometry assays.

Disclosed herein are antibodies that specifically binds to M2c macrophages with a K_{D} from 0.1 nM to 1000 nM. In some embodiments, the antibodies specifically bind to M2c macrophages with a K_{D} from about 0.1 to about 500 nM, from about 0.1 to about 100 nM, from about 0.1 to about 50 nM, from about 0.1 to about 20 nM, from about 0.1 to about 10 nM, from about 0.1 to about 5 nM, from about 0.1 to about 2 nM, from about 0.1 to about 1 nM, from about 0.1 to about 0.5 nM, from about 0.5 to about 1000 nM, from about 0.5 to about 500 nM, from about 0.5 to about 100 nM, from about 0.5 to about 50 nM, from about 0.5 to about 20 nM, from about 0.5 to about 10 nM, from about 0.5 to about 5 nM, from about 0.5 to about 2 nM, from about 0.5 to about 1 nM, from about 1 to about 1000 nM, from about 1 to about 500 nM, from about 1 to about 100 nM, from about 1 to about 50 nM, from about 1 to about 20 nM, from about 1 to about 10 nM, from about 1 to about 5 nM, from about 1 to about 2 nM, from about 2 to about 1000 nM, from about 2 to about 500 nM, from about 2 to about 100 nM, from about 2 to about 50 nM, from about 2 to about 20 nM, from about 2 to about 10 nM, from about 2 to about 5 nM, from about 5 to about 1000 nM, from about 5 to about 500 nM, from about 5 to about 100 nM, from about 5 to about 50 nM, from about 5 to about 20 nM, from about 5 to about 10 nM, from about 10 to about 1000 nM, from about 10 to about 500 nM, from about 10 to about 100 nM, from about 10 to about 50 nM, from about 10 to about 20 nM, from about 20 to about 1000 nM, from about 20 to about 500 nM, from about 20 to about 100 nM, from about 20 to about 50 nM, from about 50 to about 1000 nM, from about 50 to about 500 nM, from about 50 to about 100 nM, from about 100 to about 500 nM, from about 100 to about 1000 nM, from about 500 to about 1000 nM. In some embodiments, the antibodies specifically bind to M2c macrophages with a K_{D} of 7.7 nM.

### Binding Epitopes

Antibody epitopes may be a linear peptide sequence (i.e., “continuous”) or may be composed of noncontiguous amino acid sequences (i.e., “conformational” or “discontinuous”). In some embodiments, an antibody recognizes one or more amino acid sequences; therefore, an epitope defines more than one distinct amino acid sequence. Epitopes recognized by antibodies are determined, for example, by peptide mapping and sequence analysis techniques well known to one of skill in the art. Binding interactions are manifested as intermolecular contacts with one or more amino acid residues of a CDR.

Human CD163 protein is a protein that in humans is encoded by the CD163 gene. The amino acid sequence of human CD163 is:

(SEQ ID NO: 42)
MSKLRMVLLEDSGSADFRRHFVNLSPFTITVVLLLSACFVTSSLGGTDK
ELRLVDGENKCSGRVEVKVQEEWGTVCNNGWSMEAVSVICNQLGCPTAI
KAPGWANSSAGSGRIWMDHVSCRGNESALWDCKHDGWGKHSNCTHQQDA
GVTCSDGSNLEMRLTRGGNMCSGRIEIKFQGRWGTVCDDNFNIDHASVI
CRQLECGSAVSFSGSSNFGEGSGPIWFDDLICNGNESALWNCKHQGWGK
HNCDHAEDAGVICSKGADLSLRLVDGVTECSGRLEVRFQGEWGTICDDG
WDSYDAAVACKQLGCPTAVTAIGRVNASKGFGHIWLDSVSCQGHEPAIW
QCKHHEWGKHYCNHNEDAGVTCSDGSDLELRLRGGGSRCAGTVEVEIQR
LLGKVCDRGWGLKEADVVCRQLGCGSALKTSYQVYSKIQATNTWLFLSS
CNGNETSLWDCKNWQWGGLTCDHYEEAKITCSAHREPRLVGGDIPCSGR
VEVKHGDTWGSICDSDFSLEAASVLCRELQCGTVVSILGGAHFGEGNGQ
IWAEEFQCEGHESHLSLCPVAPRPEGTCSHSRDVGVVCSRYTEIRLVNG
KTPCEGRVELKTLGAWGSLCNSHWDIEDAHVLCQQLKCGVALSTPGGAR
FGKGNGQIWRHMFHCTGTEQHMGDCPVTALGASLCPSEQVASVICSGNQ
SQTLSSCNSSSLGPTRPTIPEESAVACIESGQLRLVNGGGRCAGRVEIY
HEGSWGTICDDSWDLSDAHVVCRQLGCGEAINATGSAHFGEGTGPIWLD
EMKCNGKESRIWQCHSHGWGQQNCRHKEDAGVICSEFMSLRLTSEASRE
ACAGRLEVFYNGAWGTVGKSSMSETTVGVVCRQLGCADKGKINPASLDK
AMSIPMWVDNVQCPKGPDTLWQCPSSPWEKRLASPSEETWITCDNKIRL
QEGPTSCSGRVEIWHGGSWGTVCDDSWDLDDAQVVCQQLGCGPALKAFK
EAEFGQGTGPIWLNEVKCKGNESSLWDCPARRWGHSECGHKEDAAVNCT
DISVQKTPQKATTGRSSRQSSFIAVGILGVVLLAIFVALFFLTKKRRQR
QRLAVSSRGENLVHQIQYREMNSCLNADDLDLMNSSGGHSEPH.

Disclosed herein are antibodies that specifically bind to an epitope in human CD163. In some embodiments, an antibody disclosed herein binds to an epitope comprising noncontiguous amino acid sequences. In some embodiments, the antibody binds to an epitope of human CD163 comprising the amino acid sequence IGRVNASKGFGHIWLDSVSCQGHEPAI (SEQ ID NO: 43). In some embodiments, the antibody binds to an epitope of human CD163 comprising the amino acid sequence VVCRQLGCGSA (SEQ ID NO: 44). In some embodiments, the antibody binds to an epitope of human CD163 comprising the amino acid sequence WDCKNWQWGGLTCD (SEQ ID NO: 45). In some embodiments, the antibody binds to an epitope of human CD163 comprising the amino acid sequences of SEQ ID NOs:43-45.

Also disclosed herein are additional antibodies that specifically bind to the epitope disclosed herein. These additional antibodies, or antigen-binding fragments thereof that specifically bind to the epitope disclosed herein can be identified using techniques known in the art. For example, a computational approach is used to design epitope-specific antibodies. Nimrod et al., Computational Design of Epitope-Specific Functional Antibodies, Cell Reports 25, 2121-2131, Nov. 20, 2018, (incorporated herein by reference). Another approach can be used to identify antibodies that bind to specific epitopes from a library of antibodies that bind to the antigen, such as the following: first incorporate noncanonical amino acids (ncAAs) p-benzoyl-L-phenylalanine (pBpa) and p-azido-L-phenylalanine (pAzF) into the target epitope and then select the antibodies that cross-link with the ncAA incorporated epitope after UV irradiation. Because cross-linking only occurs when the distance between the antibody and the epitope is close enough, this method can efficiently select antibodies that specifically bind to the target epitope. Chen et al. Epitope-directed antibody selection by site-specific photocrosslinking, Science Advances, 6(14), eaaz7825, 1 Apr. 2020 (incorporated herein by reference).

### Modifications of Antibodies

Antibodies, or antigen-binding fragments thereof, are modified, in some cases, using techniques known in the art for various purposes such as, for example, by addition of polyethylene glycol (PEG). In some embodiments, PEG modification (PEGylation) leads to one or more of improved circulation time, improved solubility, improved resistance to proteolysis, reduced antigenicity and immunogenicity, improved bioavailability, reduced toxicity, improved stability, and easier formulation.

In some cases when an antigen-binding fragment does not contain an Fc portion, an Fc portion is added to (e.g., recombinantly) the fragment, for example, to increase half-life of the antigen-binding fragment in circulation in blood when administered to a subject. Choice of an appropriate Fc region and methods of to incorporate such fragments are known in the art. Incorporating an Fc region of an IgG into a polypeptide of interest so as to increase its circulatory half-life, but so as not to lose its biological activity is accomplished, for example, by using conventional techniques known in the art. In some embodiments, Fc portions of antibodies are further modified to increase half-life of the antigen-binding fragment in circulation in blood when administered to a subject. Modifications are, for example, determined using conventional means in the art.

Additionally, in some embodiments, antibodies and antigen-binding fragments thereof are produced or expressed so that they do not contain fucose on their complex N-glycoside-linked sugar chains. The removal of the fucose from the complex N-glycoside-linked sugar chains is known to increase effector functions of the antibodies and antigen-binding fragments, including but not limited to, antibody dependent cell-mediated cytotoxicity (ADCC) and complement dependent cytotoxicity (CDC). Similarly, antibodies or antigen-binding fragments thereof that bind an epitope are, in some cases, attached at their C-terminal end to all or part of an immunoglobulin heavy chain derived from any antibody isotype, e.g., IgG, IgA, IgE, IgD, and IgM, and any of the isotype sub-classes, particularly IgG1, IgG2, IgG3, and IgG4.

Additionally, the antibodies or antigen-binding fragments described herein are also modified so that they are able to cross the blood-brain barrier in some embodiments. Such modification of the antibodies or antigen-binding fragments described herein allows for the treatment of brain diseases such as glioblastoma multiforme (GBM). Exemplary modifications to allow proteins such as antibodies or antigen-binding fragments to cross the blood-brain barrier are described in US Pat. Publ. 2007/0082380.

Glycosylation of immunoglobulins has been shown to have significant effects on their effector functions, structural stability, and rate of secretion from antibody-producing cells. The carbohydrate groups responsible for these properties are generally attached to the constant (C) regions of the antibodies. For example, glycosylation of IgG at asparagine 297 in the C_{H}2 domain is required for full capacity of IgG to activate the classical pathway of complement-dependent cytolysis (Tao and Morrison, J Immunol 143:2595 (1989)). Glycosylation of IgM at asparagine 402 in the C_{H}3 domain is necessary for proper assembly and cytolytic activity of the antibody (Muraoka and Shulman, J Immunol 142:695 (1989)). Removal of glycosylation sites as positions 162 and 419 in the CHI and C_{H}3 domains of an IgA antibody led to intracellular degradation and at least 90% inhibition of secretion (Taylor and Wall, Mol Cell Biol 8:4197 (1988)). Additionally, in some embodiments, antibodies and antigen-binding fragments thereof are produced or expressed so that they do not contain fucose on their complex N-glycoside-linked sugar chains. The removal of the fucose from the complex N-glycoside-linked sugar chains is known to increase effector functions of the antibodies and antigen-binding fragments, including but not limited to, antibody dependent cell-mediated cytotoxicity (ADCC) and complement dependent cytotoxicity (CDC). These “defucosylated” antibodies and antigen-binding fragments are produced, in some embodiments, through a variety of systems utilizing molecular cloning techniques known in the art, including but not limited to, transgenic animals, transgenic plants, or cell-lines that have been genetically engineered so that they no longer contain the enzymes and biochemical pathways necessary for the inclusion of a fucose in the complex N-glycoside-linked sugar chains (also known as fucosyltransferase knock-out animals, plants, or cells). Non-limiting examples of cells that are engineered to be fucosyltransferase knock-out cells include CHO cells, SP2/0 cells, NS0 cells, and YB2/0 cells.

Glycosylation of immunoglobulins in the variable (V) region has also been observed. Sox and Hood reported that about 20% of human antibodies are glycosylated in the V region (Proc Natl Acad Sci USA 66:975 (1970)). Glycosylation of the V domain is believed to arise from fortuitous occurrences of the N-linked glycosylation signal Asn-Xaa-Ser/Thr in the V region sequence and has not been recognized in the art as playing a role in immunoglobulin function.

Glycosylation at a variable domain framework residue, in some cases, alters the binding interaction of the antibody with antigen. The present disclosure includes criteria by which a limited number of amino acids in the framework or CDRs of a humanized immunoglobulin chain are chosen to be mutated (e.g., by substitution, deletion, or addition of residues) to increase the affinity of an antibody.

In some embodiments, cysteine residue(s) are removed or introduced in the Fc region of an antibody or Fc-containing polypeptide, thereby eliminating or increasing interchain disulfide bond formation in this region. A homodimeric specific binding agent or antibody generated using such methods, in some embodiments, exhibit improved internalization capability and/or increased complement-mediated cell killing and antibody-dependent cellular cytotoxicity (ADCC).

It has been shown that sequences within the CDR cause an antibody to bind to MHC Class II and trigger an unwanted helper T-cell response in some cases. In some embodiments, a conservative substitution allows the antibody to retain binding activity yet reduce its ability to trigger an unwanted T-cell response. In one embodiment, one or more of the N-terminal 20 amino acids of the heavy or light chain is removed.

In some embodiments, antibody molecules are produced with altered carbohydrate structure resulting in altered effector activity, including antibody molecules with absent or reduced fucosylation that exhibit improved ADCC activity. A variety of ways are known in the art to accomplish this. For example, ADCC effector activity is mediated by binding of the antibody molecule to the FcγRIII receptor, which has been shown to be dependent on the carbohydrate structure of the N-linked glycosylation at the Asn-297 of the C_{H}2 domain. Non-fucosylated antibodies bind this receptor with increased affinity and trigger FcγRIII-mediated effector functions more efficiently than native, fucosylated antibodies. Some host cell strains, e.g., Lec13 or rat hybridoma YB2/0 cell line naturally produce antibodies with lower fucosylation levels. An increase in the level of bisected carbohydrate, e.g., through recombinantly producing antibody in cells that overexpress GnTIII enzyme, has also been determined to increase ADCC activity. In some embodiments, the absence of only one of the two fucose residues are sufficient to increase ADCC activity.

Covalent modifications of an antibody are also included herein. In some embodiments, they are made by chemical synthesis or by enzymatic or chemical cleavage of the antibody, if applicable. In some embodiments, other types of covalent modifications are introduced by reacting targeted amino acid residues with an organic derivatizing agent that is capable of reacting with selected side chains or the N- or C-terminal residues.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

In some embodiments, histidyl residues are derivatized by reaction with diethylpyrocarbonate at pH 5.5-7.0 because this agent is relatively specific for the histidyl side chain. In some embodiments, para-bromophenacyl bromide also is useful; the reaction, in some embodiments, is performed in 0.1 M sodium cacodylate at pH 6.0.

In some embodiments, lysinyl and amino-terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

In some embodiments, arginyl residues are modified by reaction with one or several conventional reagents, such as phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents, in some embodiments, react with the groups of lysine as well as the arginine epsilon-amino group.

In some embodiments, the specific modification of tyrosyl residues are made, with particular interest in introducing spectral labels into tyrosyl residues by reaction with aromatic diazonium compounds or tetranitromethane. Most commonly. N-acetylimidazole and tetranitromethane are used to form O-acetyl tyrosyl species and 3-nitro derivatives, respectively, in some embodiments. Tyrosyl residues are iodinated using ¹²⁵I or ¹³¹I to prepare labeled proteins for use in radioimmunoassay.

Carboxyl side groups (aspartyl or glutamyl) are specifically modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

In some embodiments, glutaminyl and asparaginyl residues are deamidated to the corresponding glutamyl and aspartyl residues, respectively. These residues are deamidated under neutral or basic conditions.

Other modifications include hydroxylation of proline and lysine, phosphorylation of hydroxyl groups of seryl or threonyl residues, methylation of the alpha-amino groups of lysine, arginine, and histidine side chains, acetylation of the N-terminal amine, and amidation of any C-terminal carboxyl group.

Another type of covalent modification involves chemically or enzymatically coupling glycosides to the specific binding agent or antibody. These procedures do not require production of the polypeptide or antibody in a host cell that has glycosylation capabilities for N- or O-linked glycosylation. Depending on the coupling mode used, in some embodiments, the sugar(s) are attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Removal of any carbohydrate moieties present on the polypeptide or antibody are, in some embodiments, accomplished chemically or enzymatically. Chemical deglycosylation involves exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Enzymatic cleavage of carbohydrate moieties on an antibody is achieved by the use of a variety of endo- and exo-glycosidases in some embodiments.

Another type of covalent modification comprises linking an antibody to one of a variety of nonproteinaceous polymers, e.g., polyethylene glycol, polypropylene glycol, polyoxyethylated polyols, polyoxyethylated sorbitol, polyoxyethylated glucose, polyoxyethylated glycerol, polyoxyalkylenes, or polysaccharide polymers such as dextran. Such methods are known in the art.

Affinity for binding a pre-determined polypeptide antigen, generally, is modulated by introducing one or more mutations into the V region framework, typically in areas adjacent to one or more CDRs and/or in one or more framework regions. Typically, such mutations involve the introduction of conservative amino acid substitutions that either destroy or create the glycosylation site sequences but do not substantially affect the hydropathic structural properties of the polypeptide. Typically, mutations that introduce a proline residue are avoided.

### Effector Functions

Examples of antibody effector functions include: C1q binding and complement dependent cytotoxicity; Fc receptor binding; antibody-dependent cell-mediated cytotoxicity (ADCC); phagocytosis; down-regulation of cell surface receptors (e.g., B cell receptor); and B cell activation. Typically, the Fc-mediated functions involve binding of the Fc portion of the antibody by specialized receptor molecules, “Fc receptors” or “FcR,” expressed by the cell whose function is to be affected.

IgG is considered the most versatile immunoglobulin because it carries out all of the functions of immunoglobulin molecules in some embodiments. IgG is the major Ig in serum, and the only class of Ig that crosses the placenta. IgG also fixes complement, although the IgG4 subclass does not. Macrophages, monocytes, polymorphonuclear leukocytes (PMNs), and some lymphocytes have receptors for the Fc region of IgG. Not all subclasses bind equally well; IgG2 and IgG4 do not bind to Fc receptors. A consequence of binding to the Fc receptors on PMNs, monocytes, and macrophages is that the cell now internalizes the antigen better in some cases. IgG is an opsonin that enhances phagocytosis. Binding of IgG to Fc receptors on other types of cells results in the activation of other functions.

In certain embodiments, the FcR is a native sequence human FcR. Moreover, a preferred FcR is one that binds an IgG antibody (a gamma (“y”) receptor) and includes receptors of the FcγRI, FcγRII, and FcγRIII subclasses, including allelic variants and alternatively spliced forms of these receptors FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI. FcγRII, and FcγRIII. To assess ADCC activity of a molecule of interest, an in vitro ADCC assay is performed in some embodiments. Useful effector cells for such assays include peripheral blood mononuclear cells (PBMC) and Natural Killer (NK) cells.

Alternatively, or additionally, in some embodiments, ADCC activity of the molecule of interest is assessed in vivo, e.g., in an animal model.

In some embodiments, the antibodies of the disclosure bind to a surface membrane protein of and are internalized by M2-like macrophages. This internalization process is believed to be involved in the observed alteration of the functional immunosuppressive characteristics of these cells, i.e., the differentiation of the cells from M2 status to subtly activated state, without killing them or inhibiting their proliferation. In some embodiments, upon internalization, the antibodies decrease the expression of immunosuppressive soluble factors while increasing expression of soluble factors that stimulate or promote the activity or proliferation of T cells, including CD4⁺ helper T cells and cytotoxic lymphocytes.

For certain therapeutic applications, the internalization process is employed for purposes of killing or decreasing the activity or proliferation of a target cell that expresses a CD163 protein. The number of antibody molecules internalized will be sufficient or adequate to kill a cell or inhibit its growth. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the targeted cell.

In some embodiments, the antibody or antigen-binding fragment provided herein is conjugated or linked to a therapeutic moiety, an imaging or detectable moiety, or an affinity tag. Methods for conjugating or linking polypeptides are well known in the art. Associations (binding) between compounds and labels include any means known in the art including, but not limited to, covalent and non-covalent interactions, chemical conjugation, as well as recombinant techniques. An antibody or antigen-binding fragment thereof is conjugated to, or recombinantly engineered with, an affinity tag (e.g., a purification tag), in some embodiments. Affinity tags such as, for example, poly-histidine (e.g., His6; SEQ ID NO: 46) tags are conventional in the art.

In some embodiments, the antibody or antigen-binding fragment further comprises a detectable moiety. Detections accomplished, for example, in vitro, in vivo or ex vivo. In vitro assays for the detection and/or determination (quantification, qualification, etc.) of, e.g., huCD163 protein expressed by macrophages using the antibodies or antigen-binding fragments thereof include but are not limited to, for example, ELISAs, RIAs. and western blots. In some embodiments, in vitro detection, diagnosis, or monitoring of the antigen of the antibodies occurs by obtaining a sample (e.g., a blood sample) from a subject and testing the sample in, for example, a standard ELISA assay.

### Methods of Treating Cancer

Disclosed herein, in certain embodiments, are methods of treating a cancer in an individual in need thereof, comprising administering to the individual an antibody disclosed herein. In some embodiments, the disclosure provides a use of an antibody as described herein, for the manufacture of a medicament for treating cancer in a human subject. In some embodiments, the antibody specifically binds to a CD163 protein expressed on human tumor associated macrophages and reduces expression of at least one of CD16, CD64, TLR2, or Siglec-15 by the macrophages.

Disclosed herein, in certain embodiments, are methods of modulating immune activity in a subject in need thereof, comprising administering to said subject an antibody described herein. In some embodiments, the antibody specifically binds to a CD163 protein expressed on human tumor associated macrophages and reduces expression of at least one of CD16, CD64, TLR2, or Siglec-15 by the macrophages.

Disclosed herein, in certain embodiments, are methods of treating a subject with pathologically or inappropriately elevated levels of M2 macrophages (e.g., inappropriately elevated relative to the level useful for promoting immune-mediated tumor cell killing in the subject), comprising administering to said subject an antibody described herein. In some embodiments, the antibody specifically binds to a CD163 protein expressed on human tumor associated macrophages and reduces expression of at least one of CD16, CD64, TLR2, or Siglec-15 by the macrophages.

Disclosed herein, in certain embodiments, are methods of modulating an activity of a tumor-associated macrophage in a tumor microenvironment, the method comprising contacting the tumor-associated macrophage with an antibody disclosed herein, wherein the method results in at least one of the following effects:
(a) reduced expression of at least one marker by the human macrophage, wherein the at least one marker is CD16. CD64. TLR2, or Siglec-15;
(b) internalization of the antibody by the human macrophage;
(c) activation of a CD4⁺ T cell, CD8⁺ T cell, NK cell, or any combination thereof;
(d) proliferation of a CD4⁺ T cell, CD8⁺ T cell, NK cell, or any combination thereof; and (e) promotion of tumor cell killing in a tumor microenvironment.

Disclosed herein, in certain embodiments, are methods of modulating an activity of a tumor-associated macrophage in a tumor microenvironment, the method comprising contacting the tumor-associated macrophage with an antibody disclosed herein, wherein the method results in at least two of the following effects:
(a) reduced expression of at least one marker by the human macrophage, wherein the at least one marker is CD16, CD64, TLR2, or Siglec-15;
(b) internalization of the antibody by the human macrophage;
(c) activation of a CD4⁺ T cell, CD8⁺ T cell, NK cell, or any combination thereof;
(d) proliferation of a CD4⁺ T cell, CD8⁺ T cell, NK cell, or any combination thereof; and
(e) promotion of tumor cell killing in a tumor microenvironment.

Disclosed herein, in certain embodiments, are methods of modulating an activity of a tumor-associated macrophage in a tumor microenvironment, the method comprising contacting the tumor-associated macrophage with an antibody disclosed herein, wherein the method results in at least three of the following effects:
(a) reduced expression of at least one marker by the human macrophage, wherein the at least one marker is CD16, CD64, TLR2, or Siglec-15;
(b) internalization of the antibody by the human macrophage;
(c) activation of a CD4⁺ T cell, CD8+ T cell, NK cell, or any combination thereof;
(d) proliferation of a CD4⁺ T cell, CD8⁺ T cell, NK cell, or any combination thereof; and
(e) promotion of tumor cell killing in a tumor microenvironment.

Disclosed herein, in certain embodiments, are methods of modulating an activity of a tumor-associated macrophage in a tumor microenvironment, the method comprising contacting the tumor-associated macrophage with an antibody disclosed herein, wherein the method results in at least four of the following effects:
(a) reduced expression of at least one marker by the human macrophage, wherein the at least one marker is CD16, CD64, TLR2, or Siglec-15;
(b) internalization of the antibody by the human macrophage;
(c) activation of a CD4⁺ T cell, CD8⁺ T cell, NK cell, or any combination thereof;
(d) proliferation of a CD4+ T cell, CD8+ T cell, NK cell, or any combination thereof; and
(e) promotion of tumor cell killing in a tumor microenvironment.

Disclosed herein, in certain embodiments, are methods of modulating an activity of a tumor-associated macrophage in a tumor microenvironment, the method comprising contacting the tumor-associated macrophage with an antibody disclosed herein, wherein the method results in at least five of the following effects:
(a) reduced expression of at least one marker by the human macrophage, wherein the at least one marker is CD16, CD64, TLR2, or Siglec-15;
(b) internalization of the antibody by the human macrophage;
(c) activation of a CD4⁺ T cell, CD8⁺ T cell, NK cell, or any combination thereof;
(d) proliferation of a CD4⁺ T cell, CD8⁺ T cell, NK cell, or any combination thereof; and
(e) promotion of tumor cell killing in a tumor microenvironment.

Disclosed herein, in certain embodiments, are methods of functionally reorienting tumor-associated macrophages to reduce immunosuppression in a patient having cancer, comprising administering to the patient an amount of a pharmaceutical composition comprising an antibody as described herein that is effective to improve CD4⁺ or CD8⁺ T cell activity or proliferation in the tumor microenvironment.

Disclosed herein, in certain embodiments, are methods of promoting lymphocyte-mediated tumor cell killing in a human subject in need thereof, comprising administering to the subject an effective amount of a pharmaceutical composition comprising an antibody as described herein.

In some embodiments, the antibody comprises: a light chain variable domain (V_{L}) having an amino acid sequence at least about 70% identical to an amino acid sequence set forth as SEQ ID NO: 28. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 28. In some embodiments, the V_{L} has an amino acid sequence 100% identical to an amino acid sequence set forth as SEQ ID NO: 28.

In some embodiments, the antibody comprises: a light chain variable domain (V_{L}) having an amino acid sequence at least about 70% identical to an amino acid sequence set forth as SEQ ID NO: 30. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 30. In some embodiments, the V_{L} has an amino acid sequence 100% identical to an amino acid sequence set forth as SEQ ID NO: 30.

In some embodiments, the antibody comprises: a light chain variable domain (V_{L}) having an amino acid sequence at least about 70% identical to an amino acid sequence set forth as SEQ ID NO: 32. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 32. In some embodiments, the V_{L} has an amino acid sequence 100% identical to an amino acid sequence set forth as SEQ ID NO: 32.

In some embodiments, the antibody comprises: a light chain variable domain (V_{L}) having an amino acid sequence at least about 70% identical to an amino acid sequence set forth as SEQ ID NO: 34. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 34. In some embodiments, the V_{L} has an amino acid sequence 100% identical to an amino acid sequence set forth as SEQ ID NO: 34.

In some embodiments, the antibody comprises: a light chain variable domain (V_{L}) having an amino acid sequence at least about 70% identical to an amino acid sequence set forth as SEQ ID NO: 36. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 36. In some embodiments, the V_{L} has an amino acid sequence 100% identical to an amino acid sequence set forth as SEQ ID NO: 36.

In some embodiments, the antibody comprises: a light chain variable domain (V_{L}) having an amino acid sequence at least about 70% identical to an amino acid sequence set forth as SEQ ID NO: 38. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 38. In some embodiments, the V_{L} has an amino acid sequence 100% identical to an amino acid sequence set forth as SEQ ID NO: 38.

In some embodiments, the antibody comprises: a heavy chain variable domain (V_{H}) having an amino acid sequence at least about 70% identical to an amino acid sequence set forth as SEQ ID NO: 29. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 29. In some embodiments, the V_{H} has an amino acid sequence 100% identical to an amino acid sequence set forth as SEQ ID NO: 29.

In some embodiments, the antibody comprises: a heavy chain variable domain (V_{H}) having an amino acid sequence at least about 70% identical to an amino acid sequence set forth as SEQ ID NO: 31. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 31. In some embodiments, the V_{H} has an amino acid sequence 1000% identical to an amino acid sequence set forth as SEQ ID NO: 31.

In some embodiments, the antibody comprises: a heavy chain variable domain (V_{H}) having an amino acid sequence at least about 70% identical to an amino acid sequence set forth as SEQ ID NO: 33. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 33. In some embodiments, the V_{H} has an amino acid sequence 100% identical to an amino acid sequence set forth as SEQ ID NO: 33.

In some embodiments, the antibody comprises: a heavy chain variable domain (V_{H}) having an amino acid sequence at least about 70% identical to an amino acid sequence set forth as SEQ ID NO: 35. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 35. In some embodiments, the V_{H} has an amino acid sequence 100% identical to an amino acid sequence set forth as SEQ ID NO: 35.

In some embodiments, the antibody comprises: a heavy chain variable domain (V_{H}) having an amino acid sequence at least about 70% identical to an amino acid sequence set forth as SEQ ID NO: 37. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 37. In some embodiments, the V_{H} has an amino acid sequence 100% identical to an amino acid sequence set forth as SEQ ID NO: 37.

In some embodiments, the antibody comprises: a heavy chain variable domain (V_{H}) having an amino acid sequence at least about 70% identical to an amino acid sequence set forth as SEQ ID NO: 39. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 39. In some embodiments, the V_{H} has an amino acid sequence 100% identical to an amino acid sequence set forth as SEQ ID NO: 39.

In some embodiments, the antibody comprises: a light chain variable domain (V_{L}) having an amino acid sequence at least about at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence set forth in the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40; and a heavy chain variable domain (V_{H}) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence set forth as in the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; provided that the antibody does not comprise the light chain variable sequence of SEQ ID NO: 40 and heavy chain variable sequence of SEQ ID NO: 41. In some embodiments, the sequence of the antibody is 100% identical at CDR H1, CDR H2, CDR H2, CDR L1, CDR L2, and CDR L3.

In some embodiments, the antibody comprises: a light chain variable domain (V_{L}) having an amino acid sequence at least about at least about 80% identical to an amino acid sequence set forth in the group consisting of: SEQ ID NO: 28, SEQ ID NO: 30, SEQ ID NO: 32, SEQ ID NO: 34, SEQ ID NO: 36, SEQ ID NO: 38, SEQ ID NO: 40; and a heavy chain variable domain (V_{H}) having an amino acid sequence at least about 80% identical to an amino acid sequence set forth as in the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; provided that the antibody does not comprise the light chain variable sequence of SEQ ID NO: 40 and the heavy chain variable sequence of SEQ ID NO: 41. In some embodiments, the sequence of the antibody is 100% identical at CDR H1, CDR H2, CDR H2, CDR L1, CDR L2, and CDR L3.

In some embodiments, the antibody comprises: a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; provided that the antibody does not comprise a sequence as set forth in SEQ ID NO: 1, SEQ ID NO: 2, and SEQ ID NO: 3.

In some embodiments, the antibody comprises: a light chain CDR1 having an amino acid sequence 100% identical to an amino acid sequence set forth in the group consisting of: SEQ ID NO: 1, SEQ ID NO: 7, and SEQ ID NO: 13; a light chain CDR2 having an amino acid sequence 100% identical to an amino acid sequence set forth in the group consisting of: SEQ ID NO: 2, SEQ ID NO: 9, and SEQ ID NO: 14; and a light chain CDR3 having an amino acid sequence 100% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; provided that the antibody does not comprise a sequence as set forth in SEQ ID NO: 1, SEQ ID NO: 2, and SEQ ID NO: 3.

In some embodiments, the antibody comprises: a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 4, SEQ ID NO: 5, and SEQ ID NO: 6.

In some embodiments, the antibody comprises: a heavy chain CDR1 having an amino acid sequence 100% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence 100% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence 100% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 4, SEQ ID NO: 5, and SEQ ID NO: 6.

In some embodiments, the antibody comprises: (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 1, SEQ ID NO: 2, SEQ ID NO: 3, SEQ ID NO: 4, SEQ ID NO: 5, and SEQ ID NO: 6.

In some embodiments, the antibody comprises: (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 1, SEQ ID NO: 2, SEQ ID NO: 3. SEQ ID NO: 4, SEQ ID NO: 5, and SEQ ID NO: 6.

In some embodiments, the disclosure provides a use of an antibody as described herein, for the manufacture of a medicament that reduces immunosuppression by tumor-associated macrophages in a human subject having a cancer.

In some embodiments, the disclosure provides a use of an antibody as described herein, for the manufacture of a medicament that promotes T cell-mediated tumor cell killing in a human subject having a cancer.

In some embodiments, the disclosure provides a method of treating a human subject having a cancer, comprising administering to the subject a therapeutically effective amount of an antibody as described herein, whereby immunosuppression by tumor-associated macrophages in the subject is reduced.

In some embodiments, the disclosure provides a method of treating a human subject having a cancer, comprising administering to the subject a therapeutically effective amount of an antibody as described herein, whereby T cell-mediated tumor cell killing in the subject is increased.

In some embodiments, a method disclosed herein reduces myeloid cell suppression of CD8⁺ T cell activation and proliferation.

In some embodiments, the antibodies reduce myeloid cell suppression of CAR T-cell-mediated killing of cancer cells.

In some embodiments, the antibodies reduce myeloid cell suppression of NK cell-mediated killing of cancer cells by ADCC.

In some embodiments, the cancer is a lung carcinoma or sarcoma. In some embodiments, the lung cancer is a lung adenocarcinoma. In some embodiments, the lung cancer is non-small cell lung cancer.

Any of the methods disclosed herein, in some instances, further comprise administering to said subject an additional anticancer therapy. Anticancer therapies include, but are not limited to, surgical therapy, chemotherapy, radiation therapy, cryotherapy, hormonal therapy, immunotherapy, and cytokine therapy, and combinations thereof. In one embodiment, the antibody or antigen-binding fragment thereof and the anticancer therapy are administered concurrently or sequentially.

In some embodiments, the additional anticancer therapy is an immunotherapy. In some embodiments, the immunotherapy is a composition comprising a checkpoint inhibitor. In some embodiments, the additional anticancer therapy is an immune checkpoint inhibitor.

### Methods of Treating Fibrosis

Disclosed herein, in certain embodiments, are methods of treating fibrosis, such as fibrosis associated with a presence of M2-macrophages, in a subject in need thereof, comprising administering to the subject an antibody disclosed herein. In some embodiments, the fibrosis is a primary fibrotic disease. In some embodiments, the fibrosis is a secondary fibrotic disease. In some embodiments, the disclosure provides a use of an antibody as described herein, for the manufacture of a medicament for treating fibrosis in a human subject. In some embodiments, the antibody specifically binds to a CD163 protein expressed on human macrophages, such as tissue-resident or infiltrating macrophages, and reduces expression of at least one of CD16, CD64, TLR2, or Siglec-15 by the macrophages.

Disclosed herein, in certain embodiments, are methods of modulating immune activity in a subject in need thereof, comprising administering to said subject an antibody described herein. In some embodiments, the antibody specifically binds to a CD163 protein expressed on human macrophages, such as tissue-resident or infiltrating macrophages, and reduces expression of at least one of CD16, CD64, TLR2, or Siglec-15 by the macrophages.

Disclosed herein, in certain embodiments, are methods of treating a subject with pathologically or inappropriately elevated levels of M2 macrophages (e.g., inappropriately elevated relative to the level useful for promoting appropriate wound-healing and/or tissue-regeneration in the subject), comprising administering to said subject an antibody described herein. In some embodiments, the antibody specifically binds to a CD163 protein expressed on human macrophages, such as tissue-resident or infiltrating macrophages, and reduces expression of at least one of CD16, CD64, TLR2, or Siglec-15 by the macrophages.

Disclosed herein, in certain embodiments, are methods of functionally reorienting macrophages, such as tissue-resident or infiltrating M2 macrophages, to reduce pro-fibrotic functions of macrophages in a patient having fibrosis, such as fibrosis associated with a presence of M2-macrophages, comprising administering to the patient an amount of a pharmaceutical composition comprising an antibody as described herein that is effective to reduce activation and/or proliferation of fibroblasts in a fibrotic tissue. In some embodiments, the fibrosis is a primary fibrotic disease. In some embodiments, the fibrosis is a secondary fibrotic disease.

Disclosed herein, in certain embodiments, are methods of reducing secretion of TGF-β, PDGF, VEGF, IGF-1, Galectin-3, IL-10, or combinations thereof by macrophages, such as tissue-resident or infiltrating M2 macrophages, in a human subject in need thereof, comprising administering to the subject an effective amount of a pharmaceutical composition comprising an antibody as described herein.

Disclosed herein, in certain embodiments, are methods of modulating an activity of a macrophage, such as an M2 macrophage, in a fibrotic tissue, the method comprising contacting the macrophage with an antibody disclosed herein, wherein the method results in at least one of the following effects:
(a) reduced expression of at least one marker by the human macrophage, wherein the at least one marker is CD16, CD64, TLR2, or Siglec-15; or
(b) internalization of the antibody by the human macrophage.
   In some embodiments, the fibrosis is a primary fibrotic disease. In some embodiments, the fibrosis is a secondary fibrotic disease.

In some embodiments, the antibody comprises: a light chain variable domain (V_{L}) having an amino acid sequence at least about 70% identical to an amino acid sequence set forth as SEQ ID NO: 28. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 28. In some embodiments, the V_{L} has an amino acid sequence 100% identical to an amino acid sequence set forth as SEQ ID NO: 28.

In some embodiments, the antibody comprises: a light chain variable domain (V_{L}) having an amino acid sequence at least about 70% identical to an amino acid sequence set forth as SEQ ID NO: 30. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 30. In some embodiments, the V_{L} has an amino acid sequence 100% identical to an amino acid sequence set forth as SEQ ID NO: 30.

In some embodiments, the antibody comprises: a light chain variable domain (V_{L}) having an amino acid sequence at least about 70% identical to an amino acid sequence set forth as SEQ ID NO: 32. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 32. In some embodiments, the V_{L} has an amino acid sequence 100% identical to an amino acid sequence set forth as SEQ ID NO: 32.

In some embodiments, the antibody comprises: a light chain variable domain (V_{L}) having an amino acid sequence at least about 70% identical to an amino acid sequence set forth as SEQ ID NO: 34. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 34. In some embodiments, the V_{L} has an amino acid sequence 100% identical to an amino acid sequence set forth as SEQ ID NO: 34.

In some embodiments, the antibody comprises: a light chain variable domain (V_{L}) having an amino acid sequence at least about 70% identical to an amino acid sequence set forth as SEQ ID NO: 36. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 36. In some embodiments, the V_{L} has an amino acid sequence 100% identical to an amino acid sequence set forth as SEQ ID NO: 36.

In some embodiments, the antibody comprises: a light chain variable domain (V_{L}) having an amino acid sequence at least about 70% identical to an amino acid sequence set forth as SEQ ID NO: 38. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 38. In some embodiments, the V_{L} has an amino acid sequence 100% identical to an amino acid sequence set forth as SEQ ID NO: 38.

In some embodiments, the antibody comprises: a heavy chain variable domain (V_{H}) having an amino acid sequence at least about 70% identical to an amino acid sequence set forth as SEQ ID NO: 29. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 29. In some embodiments, the V_{H} has an amino acid sequence 100% identical to an amino acid sequence set forth as SEQ ID NO: 29.

In some embodiments, the antibody comprises: a heavy chain variable domain (V_{H}) having an amino acid sequence at least about 70% identical to an amino acid sequence set forth as SEQ ID NO: 31. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 31. In some embodiments, the V_{H} has an amino acid sequence 1000% identical to an amino acid sequence set forth as SEQ ID NO: 31.

In some embodiments, the antibody comprises: a heavy chain variable domain (V_{H}) having an amino acid sequence at least about 70% identical to an amino acid sequence set forth as SEQ ID NO: 33. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 33. In some embodiments, the V_{H} has an amino acid sequence 1000% identical to an amino acid sequence set forth as SEQ ID NO: 33.

In some embodiments, the antibody comprises: a heavy chain variable domain (V_{H}) having an amino acid sequence at least about 70% identical to an amino acid sequence set forth as SEQ ID NO: 35. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 35. In some embodiments, the V_{H} has an amino acid sequence 100% identical to an amino acid sequence set forth as SEQ ID NO: 35.

In some embodiments, the antibody comprises: a heavy chain variable domain (V_{H}) having an amino acid sequence at least about 70% identical to an amino acid sequence set forth as SEQ ID NO: 37. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 37. In some embodiments, the V_{H} has an amino acid sequence 100% identical to an amino acid sequence set forth as SEQ ID NO: 37.

In some embodiments, the antibody comprises: a heavy chain variable domain (V_{H}) having an amino acid sequence at least about 70% identical to an amino acid sequence set forth as SEQ ID NO: 39. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 39. In some embodiments, the V_{H} has an amino acid sequence 100% identical to an amino acid sequence set forth as SEQ ID NO: 39.

In some embodiments, the antibody comprises: a light chain variable domain (V_{L}) having an amino acid sequence at least about at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence set forth in the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40; and a heavy chain variable domain (V_{H}) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence set forth as in the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; provided that the antibody does not comprise the light chain variable sequence of SEQ ID NO: 40 and heavy chain variable sequence of SEQ ID NO. 41. In some embodiments, the sequence of the antibody is 100% identical at CDR H1, CDR H2, CDR H2, CDR L1, CDR L2, and CDR L3.

In some embodiments, the antibody comprises: a light chain variable domain (V_{L}) having an amino acid sequence at least about at least about 80% identical to an amino acid sequence set forth in the group consisting of: SEQ ID NO: 28, SEQ ID NO: 30, SEQ ID NO: 32, SEQ ID NO: 34, SEQ ID NO: 36, SEQ ID NO: 38, SEQ ID NO: 40; and a heavy chain variable domain (V_{H}) having an amino acid sequence at least about 80% identical to an amino acid sequence set forth as in the group consisting of: SEQ ID NO: 29, SEQ ID NO: 31, SEQ ID NO: 33, SEQ ID NO. 35, SEQ ID NO: 37, SEQ ID NO: 39, and SEQ ID NO: 41; provided that the antibody does not comprise the light chain variable sequence of SEQ ID NO: 40 and the heavy chain variable sequence of SEQ ID NO: 41. In some embodiments, the sequence of the antibody is 100% identical at CDR H1, CDR H2, CDR H2, CDR L1, CDR L2, and CDR L3.

In some embodiments, the antibody comprises: a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2. SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90/o, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; provided that the antibody does not comprise a sequence as set forth in SEQ ID NO: 1, SEQ ID NO: 2, and SEQ ID NO: 3.

In some embodiments, the antibody comprises: a light chain CDR1 having an amino acid sequence 100% identical to an amino acid sequence set forth in the group consisting of: SEQ ID NO: 1, SEQ ID NO: 7, and SEQ ID NO: 13; a light chain CDR2 having an amino acid sequence 100% identical to an amino acid sequence set forth in the group consisting of: SEQ ID NO: 2, SEQ ID NO: 9, and SEQ ID NO: 14; and a light chain CDR3 having an amino acid sequence 100% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; provided that the antibody does not comprise a sequence as set forth in SEQ ID NO: 1, SEQ ID NO: 2, and SEQ ID NO: 3.

In some embodiments, the antibody comprises: a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 4, SEQ ID NO: 5, and SEQ ID NO: 6.

In some embodiments, the antibody comprises: a heavy chain CDR1 having an amino acid sequence 100% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence 100% identical to an amino acid sequence set forth in the group consisting of: SEQ ID NO: 5, SEQ ID NO: 17, SEQ ID NO: 20, SEQ ID NO; 23, and SEQ ID NO: 26; a heavy chain CDR3 having an amino acid sequence 100% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 4, SEQ ID NO: 5, and SEQ ID NO: 6.

In some embodiments, the antibody comprises: (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13, a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91% 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO; 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO; 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO; 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 1, SEQ ID NO: 2, SEQ ID NO: 3, SEQ ID NO: 4, SEQ ID NO: 5, and SEQ ID NO: 6.

In some embodiments, the antibody comprises: (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 1, SEQ ID NO: 2, SEQ ID NO: 3. SEQ ID NO: 4, SEQ ID NO: 5, and SEQ ID NO: 6.

In some embodiments, the disclosure provides a use of an antibody as described herein, for the manufacture of a medicament that reduces pro-fibrotic functions by macrophages, such as tissue-resident or infiltrating M2 macrophages, in a human subject having fibrosis, such as fibrosis associated with a presence of M2-macrophages. In some embodiments, the fibrosis is a primary fibrotic disease. In some embodiments, the fibrosis is a secondary fibrotic disease.

In some embodiments, the disclosure provides a use of an antibody as described herein, for the manufacture of a medicament that reduces activation and/or proliferation of fibroblasts in a human subject having fibrosis, such as fibrosis associated with a presence of M2-macrophages. In some embodiments, the fibrosis is a primary fibrotic disease. In some embodiments, the fibrosis is a secondary fibrotic disease.

In some embodiments, the disclosure provides a method of treating a human subject having fibrosis, such as fibrosis associated with a presence of M2-macrophages, comprising administering to the subject a therapeutically effective amount of an antibody as described herein, whereby a pro-fibrotic function of macrophages, such as tissue-resident or infiltrating M2 macrophages, in the subject is reduced. In some embodiments, the fibrosis is a primary fibrotic disease. In some embodiments, the fibrosis is a secondary fibrotic disease.

In some embodiments, the disclosure provides a method of treating a human subject having fibrosis, such as fibrosis associated with a presence of M2-macrophages, comprising administering to the subject a therapeutically effective amount of an antibody as described herein, whereby activation and/or proliferation of fibroblasts is reduced. In some embodiments, the fibrosis is a primary fibrotic disease. In some embodiments, the fibrosis is a secondary fibrotic disease.

In some embodiments, the disclosure provides a method of treating a human patient having fibrosis, such as fibrosis associated with a presence of M2-macrophages, comprising administering an effective amount of an antibody as described herein to the patient.

In some embodiments, the fibrosis is a primary fibrotic disease. In some embodiments, the primary fibrotic disease is cystic fibrosis, idiopathic pulmonary fibrosis, hepatic cirrhosis, systemic sclerosis (SSc), sclerodermatous graft vs. host disease (GVHD), nephrogenic systemic fibrosis, and radiation fibrosis. Exemplary radiation fibrosis includes, for example, lung fibrosis resulting from the tissue repair that follows inflammation of the lungs (pneumonitis) caused by radiation therapy. Other soft tissues subjected to radiation therapy, e.g., for treatment of cancer, can also exhibit radiation fibrosis, e.g., breast tissue, head and neck tissue, nerves, heart tissue, blood vessels, bones, as well as muscles, tendons, and ligaments. “Radiation fibrosis syndrome” may be used to refer to clinical manifestations of progressive fibrotic tissue sclerosis resulting from radiation treatment.

In some embodiments, the fibrosis is a secondary fibrotic disease. In some embodiments, the fibrosis is a sequela of an acute disease or a chronic disease. In some embodiments, the disease or disorder for which fibrosis is a sequela is a cancer, a viral infection, or an autoimmune or inflammatory disease. Exemplary infections associated with fibrosis are sepsis, an HIV infection, a SARS-CoV-2 infection, and malaria. Exemplary autoimmune and inflammatory diseases and disorders associated with fibrosis are sickle cell disease, Type I diabetes mellitus, Type 2 diabetes mellitus, Crohn's disease, celiac disease, asthma, sarcoidosis, glomerulonephritis, lupus nephritis, systemic lupus erythematosus, rheumatoid arthritis, Sjögren's Syndrome, scleroderma, cystic fibrosis (CF), graft-versus-host disease, allograft rejection, sarcoidosis, hemophagocytic lymphohistiocytosis (HLH), inflammatory arthritis, chronic obstructive pulmonary disease (COPD), asthma, osteoarthritis, and multiple sclerosis.

In some embodiments, the fibrosis is pulmonary fibrosis. Pulmonary fibrosis can be associated with a lung disease or disorder. An exemplary lung disease is idiopathic pulmonary fibrosis (IPF). Other exemplary lung diseases and disorders include, but are not limited to, interstitial lung disease (ILD), diffuse interstitial lung disease, pulmonary sarcoidosis, acute lung injury (ALI), acute respiratory distress syndrome (ARDS), Covid-19, and hypersensitivity pneumonitis.

In some embodiments, the fibrosis is cardiac fibrosis. Cardiac fibrosis can be associated with a cardiac disease or disorder. Exemplary heart diseases and disorders include, but are not limited to, atherosclerosis, atrial fibrillation, chronic heart failure, peripheral artery disease, and acute coronary syndromes.

In some embodiments, the fibrosis is hepatic fibrosis. Hepatic fibrosis can be associated with a liver disease or disorder. Exemplary liver diseases and disorders include, but are not limited to liver fibrosis, non-alcoholic fatty liver disease (NAFLD), acute-on-chronic liver failure, acute liver failure, alcoholic hepatitis, non-alcoholic steatohepatitis, cirrhosis, and viral hepatitis.

In some embodiments, the fibrosis is renal fibrosis. Renal fibrosis can be associated with a kidney disease or disorder. Exemplary kidney diseases and disorders include, but are not limited to, an acute kidney injury, acute tubular necrosis, chronic kidney disease, and kidney allograft rejection.

In some embodiments, the fibrosis is retinal fibrosis.

In some embodiments, any of the methods disclosed herein further comprise administering to said subject an additional anti-fibrosis therapy. Anti-fibrosis therapies include, but are not limited to, nintedanib, pirfenidone, corticosteroids (e.g., prednisone), mycophenolate mofetil/mycophenolic acid, azathioprine, ACE inhibitors (e.g., benazepril, Lisinopril, and ramipril), Angiotensin II receptor blockers (ARBs), anti-viral agents (e.g., hepatitis C therapies), and TGF-β inhibitors.

In some embodiments, any of the methods disclosed herein further comprise administering to said subject an additional anti-inflammatory therapy. Anti-inflammatory therapies include, but are not limited to, corticosteroids (e.g., prednisone), nonsteroidal anti-inflammatory drugs (NSAIDs, e.g., ibuprofen), pirfenidone, and other immunomodulatory agents.

In some embodiments, other antibodies, small molecule therapeutics, and/or other agents are combined in separate compositions for simultaneous or sequential administration. In one embodiment, simultaneous administration comprises one or more compositions that are administered at the same time, or within 30 minutes of each other. In some embodiments, administration occurs at the same or different sites.

Toxicity and therapeutic efficacy of such ingredient are, in some embodiments, determined by standard pharmaceutical procedures in cell cultures or experimental animals, e.g., for determining the LD₅₀ (the dose lethal to 50% of the population) and the ED₅₀ (the dose therapeutically effective in 50% of the population). In some embodiments, the dose ratio between toxic and therapeutic effects is the therapeutic index and it are expressed as the ratio LD₅₀/ED₅₀. While compounds that exhibit toxic side effects are used in some embodiments, care should be taken to design a delivery system that targets such compounds to the site of affected tissue to minimize potential damage to healthy cells and, thereby, reduce side effects.

Data obtained from cell culture assays and animal studies is used in formulating a range of dosage for use in humans in some embodiments. The dosage of such compounds lies preferably within a range of circulating concentrations that include the ED₅₀ with little or no toxicity. In some embodiments, the dosage varies within this range depending upon the dosage form employed and the route of administration utilized. For any compound used in the method of the disclosure, the therapeutically effective dose is estimated initially from cell culture assays in some embodiments. In some embodiments, a dose is formulated in animal models to achieve a circulating plasma concentration arrange that includes the IC₅₀ (i.e., the concentration of the test compound which achieves a half-maximal inhibition) as determined in cell culture. Levels in plasma are measured, for example, by high performance liquid chromatography. Such information is, in some cases, used to more accurately determine useful doses in humans.

In some embodiments the disclosure provides a method of treating a patient having fibrosis, such as fibrosis associated with a presence of M2-macrophages, comprising administering to the patient a therapeutically effective amount of an antibody as described herein and further comprising treating the subject with fibrosis therapy selected from surgical therapy or cytokine therapy. In some embodiments, the fibrosis is a primary fibrotic disease. In some embodiments, the fibrosis is a secondary fibrotic disease. In some embodiments, the antibody, or antigen-binding fragment thereof, and another fibrosis therapy (e.g., an anti-inflammatory) are administered concurrently or sequentially.

### Diagnostic Products and Methods

In some embodiments, disclosed herein, are methods of detection of a huCD163 protein or M2 macrophages in a sample or a subject to assess a treatment state of a patient or diagnose a disease or disorder associated or correlated with the activity of M2 macrophages or tissue-resident macrophages, or infiltrating macrophages, such as those fibrotic diseases and disorders described herein.

In the in vivo detection, diagnosis or monitoring of soluble huCD163 protein, expression of a huCD163 protein by cells or tissues, or presence or activity of M2 macrophages, a subject is administered an antibody or antigen-binding fragment as described herein, which antibody or antigen-binding fragment is bound to a detectable moiety. The detectable moiety is visualized, in some embodiments, using art-recognized methods such as, but not limited to, magnetic resonance imaging (MRI), fluorescence, radioimaging, light sources supplied by endoscopes, laparoscopes, or intravascular catheter (i.e., via detection of photoactive agents), photoscanning, positron emission tomography (PET) scanning, whole body nuclear magnetic resonance (NMR), radioscintigraphy, single photon emission computed tomography (SPECT), targeted near infrared region (NIR) scanning, X-ray, ultrasound. Labels for detecting compounds using such methods are also known in the art. Visualization of the detectable moiety allows, in some embodiments, for detection, diagnosis, and/or monitoring of a condition or disease associated with M2 macrophage activity or activity of another cell that expresses a huCD163 protein. Additional diagnostic assays that utilize antibodies specific to the desired target protein, i.e., a huCD163 protein, are known in the art and are also contemplated herein.

For in vitro detection methods, samples to be obtained from a subject include, but are not limited to, blood, tissue biopsy samples, and fluid therefrom.

Thus, the disclosure provides antibodies and antigen-binding fragments thereof that are useful for detecting or diagnosing levels of M2 macrophages, tissue-resident macrophages, or infiltrating macrophages associated with a fibrotic disease or disorder, potentially indicating need for therapeutic treatment. In other embodiments the antibody further comprises a second agent. Such an agent, in some embodiments, is a molecule or moiety such as, for example, a reporter molecule or a detectable label. Detectable labels/moieties for such detection methods are known in the art and are described in more detail below. Reporter molecules are any moiety which are detected using an assay, for example. Non-limiting examples of reporter molecules which have been conjugated to polypeptides include enzymes, radiolabels, haptens, fluorescent labels, phosphorescent molecules, chemiluminescent molecules, chromophores, luminescent molecules, photoaffinity molecules, colored particles or ligands, such as biotin. In some embodiments, detectable labels include compounds and/or elements that are detected due to their specific functional properties, and/or chemical characteristics, the use of which allows the polypeptide to which they are attached to be detected, and/or further quantified if desired. Many appropriate detectable (imaging) agents are known in the art, as are methods for their attachment to polypeptides.

Polypeptides are conjugated to a wide variety of fluorescent dyes, quenchers, and haptens such as fluorescein, R-phycoerythrin, and biotin in some embodiments. In some embodiments, conjugation occurs either during polypeptide synthesis or after the polypeptide has been synthesized and purified.

Alternatively, an antibody, antigen-binding fragment or binding protein is conjugated with a fluorescent moiety in some embodiments. Conjugating polypeptides with fluorescent moieties (e.g., R-Phycoerythrin, fluorescein isothiocyanate (FITC), etc.) is, for example, accomplished using art-recognized techniques. Numerous commercially available fluorescent dyes and dye-conjugation kits are commercially available for particular applications, such as fluorescence microscopy, flow cytometry, fluorescence-activated cell sorting (FACS), etc.

In one non-limiting embodiment, an antibody antigen-binding fragment is associated with (conjugated to) a detectable label, such as a radionuclide, a dye, an imaging agent or a fluorescent agent for immunodetection of binding to antigen which is used to visualize binding of the antibodies to M2 macrophages or soluble or bound huCD163 protein in vitro and/or in vivo.

Non-limiting examples of radiolabels include, for example, ³²P, ³³P, ⁴³K, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81m}Kr, ^{87m}Sr, ⁹⁰Y, ⁹⁷Ru, ⁹⁹Tc, ^{99m}Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir ¹⁹⁷Hg, ¹⁹⁹Au. ²⁰³Pb, ²¹¹At, ²¹²Pb, ²¹²Bi, and ²¹³Bi. In some embodiments, radiolabels are attached to compounds using conventional chemistry known in the art of antibody imaging. Radiolabeled compounds are useful in in vitro diagnostics techniques and in in vivo radioimaging techniques and in radioimmunotherapy.

Compositions of antibodies and antigen-binding fragments described herein are also used as non-therapeutic agents (e.g., as affinity purification agents) in some embodiments.

### Pharmaceutical Compositions and Administration

Disclosed herein, in certain embodiments, are pharmaceutical compositions comprising an antibody as disclosed herein and a pharmaceutically acceptable excipient.

In some embodiments, the antibody comprises: a light chain variable domain (V_{L}) having an amino acid sequence at least about 70% identical to an amino acid sequence set forth as SEQ ID NO: 28. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 28. In some embodiments, the V_{L} has an amino acid sequence 100% identical to an amino acid sequence set forth as SEQ ID NO; 28.

In some embodiments, the antibody comprises: a light chain variable domain (V_{L}) having an amino acid sequence at least about 70% identical to an amino acid sequence set forth as SEQ ID NO: 30. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 30. In some embodiments, the V_{L} has an amino acid sequence 100% identical to an amino acid sequence set forth as SEQ ID NO: 30.

In some embodiments, the antibody comprises: a light chain variable domain (V_{L}) having an amino acid sequence at least about 70% identical to an amino acid sequence set forth as SEQ ID NO: 32. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 32. In some embodiments, the V_{L} has an amino acid sequence 100% identical to an amino acid sequence set forth as SEQ ID NO: 32.

In some embodiments, the antibody comprises: a light chain variable domain (V_{L}) having an amino acid sequence at least about 70% identical to an amino acid sequence set forth as SEQ ID NO: 34. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 34. In some embodiments, the V_{L} has an amino acid sequence 100% identical to an amino acid sequence set forth as SEQ ID NO: 34.

In some embodiments, the antibody comprises: a light chain variable domain (V_{L}) having an amino acid sequence at least about 70% identical to an amino acid sequence set forth as SEQ ID NO: 36. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 36. In some embodiments, the V_{L} has an amino acid sequence 100% identical to an amino acid sequence set forth as SEQ ID NO: 36.

In some embodiments, the antibody comprises: a light chain variable domain (V_{L}) having an amino acid sequence at least about 70% identical to an amino acid sequence set forth as SEQ ID NO: 38. In some embodiments the V_{L} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 38. In some embodiments, the V_{L} has an amino acid sequence 100% identical to an amino acid sequence set forth as SEQ ID NO: 38.

In some embodiments, the antibody comprises: a heavy chain variable domain (V_{H}) having an amino acid sequence at least about 70% identical to an amino acid sequence set forth as SEQ ID NO: 29. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 29. In some embodiments, the V_{H} has an amino acid sequence 100% identical to an amino acid sequence set forth as SEQ ID NO: 29.

In some embodiments, the antibody comprises: a heavy chain variable domain (V_{H}) having an amino acid sequence at least about 70% identical to an amino acid sequence set forth as SEQ ID NO: 31. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 31. In some embodiments, the V_{H} has an amino acid sequence 100% identical to an amino acid sequence set forth as SEQ ID NO: 31.

In some embodiments, the antibody comprises: a heavy chain variable domain (V_{H}) having an amino acid sequence at least about 70% identical to an amino acid sequence set forth as SEQ ID NO: 33. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 33. In some embodiments, the V_{H} has an amino acid sequence 100% identical to an amino acid sequence set forth as SEQ ID NO: 33.

In some embodiments, the antibody comprises: a heavy chain variable domain (V_{H}) having an amino acid sequence at least about 70% identical to an amino acid sequence set forth as SEQ ID NO: 35. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 35. In some embodiments, the V_{H} has an amino acid sequence 100% identical to an amino acid sequence set forth as SEQ ID NO: 35.

In some embodiments, the antibody comprises: a heavy chain variable domain (V_{H}) having an amino acid sequence at least about 70% identical to an amino acid sequence set forth as SEQ ID NO: 37. In some embodiments the V_{H} has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 37. In some embodiments, the V_{H} has an amino acid sequence 100% identical to an amino acid sequence set forth as SEQ ID NO: 37.

In some embodiments, the antibody comprises: a heavy chain variable domain (V_{H}) having an amino acid sequence at least about 70% identical to an amino acid sequence set forth as SEQ ID NO: 39. In some embodiments the V_{H} has an amino acid sequence at least about 75%, 80%, 81%, 82%, 83%, 84%, 85%, 86%, 87%, 88%, 89%, 90, 91%, 92%, 93%, 94%, 95%, 96%, 97%, 98%, or 99% identical to an amino acid sequence set forth as SEQ ID NO: 39. In some embodiments, the V_{H} has an amino acid sequence 1000% identical to an amino acid sequence set forth as SEQ ID NO: 39.

In some embodiments, the antibody comprises: a light chain variable domain (V_{L}) having an amino acid sequence at least about at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence set forth in the group consisting of: SEQ ID NO: 28, SEQ ID NO: 30, SEQ ID NO: 32, SEQ ID NO: 34, SEQ ID NO: 36, SEQ ID NO: 38, and SEQ ID NO: 40; and a heavy chain variable domain (V_{H}) having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence set forth as in the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; provided that the antibody does not comprise the light chain variable sequence of SEQ ID NO: 40 and heavy chain variable sequence of SEQ ID NO: 41. In some embodiments, the sequence of the antibody is 100% identical at CDR H1. CDR H2, CDR H2. CDR L1, CDR L2, and CDR L3.

In some embodiments, the antibody comprises: a light chain variable domain (V_{L}) having an amino acid sequence at least about at least about 80% identical to an amino acid sequence set forth in the group consisting of: SEQ ID NO: 28, SEQ ID NO: 30, SEQ ID NO: 32, SEQ ID NO: 34, SEQ ID NO: 36, SEQ ID NO: 38, SEQ ID NO: 40; and a heavy chain variable domain (VII) having an amino acid sequence at least about 80% identical to an amino acid sequence set forth as in the group consisting of: SEQ ID NO: 29, SEQ ID NO: 31, SEQ ID NO: 33, SEQ ID NO: 35, SEQ ID NO: 37, SEQ ID NO: 39, and SEQ ID NO: 41; provided that the antibody does not comprise the light chain variable sequence of SEQ ID NO; 40 and the heavy chain variable sequence of SEQ ID NO: 41. In some embodiments, the sequence of the antibody is 100% identical at CDR H1, CDR H2, CDR H2, CDR L1, CDR L2, and CDR L3.

In some embodiments, the antibody comprises: a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; provided that the antibody does not comprise a sequence as set forth in SEQ ID NO: 1, SEQ ID NO: 2, and SEQ ID NO: 3.

In some embodiments, the antibody comprises: a light chain CDR1 having an amino acid sequence 100% identical to an amino acid sequence set forth in the group consisting of: SEQ ID NO: 1, SEQ ID NO: 7, and SEQ ID NO: 13; a light chain CDR2 having an amino acid sequence 100% identical to an amino acid sequence set forth in the group consisting of: SEQ ID NO; 2, SEQ ID NO: 9, and SEQ ID NO: 14; and a light chain CDR3 having an amino acid sequence 100% identical to an amino acid sequence set forth in the group consisting of: SEQ ID NO: 3, SEQ ID NO: 8, SEQ ID NO; 10, SEQ ID NO: 11, SEQ ID NO; 12, and SEQ ID NO: 15; provided that the antibody does not comprise a sequence as set forth in SEQ ID NO: 1, SEQ ID NO: 2, and SEQ ID NO: 3.

In some embodiments, the antibody comprises: a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%. 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 4, SEQ ID NO: 5, and SEQ ID NO: 6.

In some embodiments, the antibody comprises: a heavy chain CDR1 having an amino acid sequence 100% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence 100% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence 100% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 4, SEQ ID NO: 5, and SEQ ID NO: 6.

In some embodiments, the antibody comprises: (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%. 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6, SEQ ID NO: 18, SEQ ID NO: 21, SEQ ID NO: 24, and SEQ ID NO: 27; provided that the antibody does not comprise a sequence as set forth in SEQ ID NO: 1, SEQ ID NO: 2, SEQ ID NO: 3, SEQ ID NO: 4, SEQ ID NO: 5, and SEQ ID NO: 6.

In some embodiments, the antibody comprises: (a) a light chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 1, SEQ ID NO: 7, and SEQ ID NO: 13; a light chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 2, SEQ ID NO: 9, and SEQ ID NO: 14; and a light chain CDR3 having an amino acid sequence at least about 70%, 75%, 80%/o, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and (b) a heavy chain CDR1 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the group consisting of: SEQ ID NO: 6. SEQ ID NO: 18, SEQ ID NO: 21, SEQ ID NO: 24, and SEQ ID NO: 27; provided that the antibody does not comprise a sequence as set forth in SEQ ID NO: 1, SEQ ID NO: 2, SEQ ID NO: 3, SEQ ID NO: 4, SEQ ID NO: 5, and SEQ ID NO: 6.

Such compositions are useful for in vitro or in vivo analysis or, in the case of pharmaceutical compositions, for administration to a subject in vivo or ex vivo for treating a subject with the disclosed antibodies.

In some embodiments, the excipient is a carrier, buffer, stabilizer or other suitable materials known to those skilled in the art. Such materials should be non-toxic and should not interfere with the efficacy of the active ingredient. The precise nature of the carrier or other material will depend on the route of administration.

Pharmaceutical formulations comprising an antibody or antigen-binding fragment, identified by the methods described herein are prepared for storage by mixing the protein having the desired degree of purity with optional physiologically acceptable carriers, excipients or stabilizers (see, e.g., Remington's Pharmaceutical Sciences, 16^{th} edition. Osol, A. Ed. (1980)), in the form of lyophilized formulations or aqueous solutions in some embodiments. Acceptable carriers, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG). In certain embodiments, the pharmaceutical composition comprises the antibody at a concentration of between 5-200 mg/mL; preferably between 10-100 mg/mL.

Acceptable carriers are physiologically acceptable to the administered subject and retain the therapeutic properties of the compounds with/in which it is administered. Acceptable carriers and their formulations are and generally described in, for example, Remington's Pharmaceutical Sciences, supra. One exemplary carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier is acceptable in the sense of being compatible with the other ingredients of the formulation and not injurious to a subject to whom it is administered. Nor should an acceptable carrier alter the specific activity of the subject compounds.

In another embodiment, a pharmaceutical composition disclosed herein further comprises an acceptable additive to improve the stability of the compounds in composition and/or to control the release rate of the composition. Acceptable additives do not alter the specific activity of the subject compounds. Exemplary acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives are combined with acceptable carriers and/or excipients such as dextrose in some embodiments. Alternatively, exemplary acceptable additives include, but are not limited to, a surfactant such as polysorbate 20 or polysorbate 80 to increase stability of the peptide and decrease gelling of the solution. In some embodiments, the surfactant is added to the composition in an amount of 0.01% to 5% of the solution. Addition of such acceptable additives increases the stability and half-life of the composition in storage.

In one embodiment, a pharmaceutical composition disclosed herein contains an isotonic buffer such as a phosphate, acetate, or TRIS buffer in combination with a tonicity agent such as a polyol, Sorbitol, sucrose or sodium chloride, which tonicifies and stabilizes. In some embodiments, a tonicity agent is present in the composition in an amount of about 5%.

In another embodiment, a pharmaceutical composition disclosed herein includes a surfactant such as to prevent aggregation and for stabilization at 0.01 to 0.02% wt/vol.

In another embodiment, the pH of a pharmaceutical composition disclosed herein ranges from 4.5-6.5 or 4.5-5.5.

In some embodiments, a pharmaceutical composition disclosed herein also contains more than one active compound as necessary for the indication being treated, such as those with complementary activities that do not adversely affect each other. For example, a method of treatment further provides an immunosuppressive agent. Such molecules are suitably present in combination in amounts that are effective for the purpose intended.

In some embodiments, active ingredients are entrapped in microcapsule prepared, for example, by coacervation techniques or by interfacial polymerization, for example, hydroxy methylcellulose or gelatin-microcapsule and poly-(methylmethacrylate) microcapsule, respectively, in colloidal drug delivery systems (for example, liposomes, albumin microspheres, microemulsions, nanoparticles, and nanocapsules) or in macroemulsions. Such techniques are disclosed in Remington's Pharmaceutical Sciences, supra.

Suspensions and crystal forms of antibodies are also contemplated herein; methods to make suspensions and crystal forms are known to one of skill in the art.

In some embodiments, a pharmaceutical composition disclosed herein is sterile. In some embodiments, a pharmaceutical composition disclosed herein is sterilized by conventional, well known sterilization techniques. For example, sterilization is readily accomplished by filtration through sterile filtration membranes. In some embodiments, the resulting solutions is packaged for use or filtered under aseptic conditions and lyophilized, the lyophilized preparation being combined with a sterile solution prior to administration.

Freeze-drying is employed to stabilize polypeptides for long-term storage, such as when a polypeptide is relatively unstable in liquid compositions, in some embodiments.

In some embodiments, some excipients such as, for example, polyols (including mannitol, sorbitol, and glycerol); sugars (including glucose and sucrose); and amino acids (including alanine, glycine, and glutamic acid), act as stabilizers for freeze-dried products. Polyols and sugars are also used to protect polypeptides from freezing and drying-induced damage and to enhance the stability during storage in the dried state in some embodiments. Sugars are, in some embodiments, effective in both the freeze-drying process and during storage. Other classes of molecules, including mono- and disaccharides and polymers such as PVP, have also been reported as stabilizers of lyophilized products.

For injection, in some embodiments, a pharmaceutical composition disclosed herein is a powder suitable for reconstitution with an appropriate solution as described above. Examples of these include, but are not limited to, freeze dried, rotary dried or spray dried powders, amorphous powders, granules, precipitates, or particulates. For injection, the compositions optionally contain stabilizers, pH modifiers, surfactants, bioavailability modifiers and combinations of these.

Sustained-release preparations is prepared in some embodiments. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (see, e.g., U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. In some embodiments, while encapsulated antibodies remain in the body for a long time, they denature or aggregate as a result of exposure to moisture at 37° C., resulting in a loss of biological activity and possible changes in immunogenicity. Rational strategies devised for stabilization are, in some cases, dependent on the mechanism involved. For example, if the aggregation mechanism is discovered to be intermolecular S—S bond formation through thio-disulfide interchange, stabilization is achieved, in some cases, by modifying sulfhydryl residues, lyophilizing from acidic solutions, controlling moisture content, using appropriate additives, and developing specific polymer matrix compositions.

In some embodiments, a pharmaceutical composition disclosed herein is designed to be short-acting, fast-releasing, long-acting, or sustained-releasing as described herein. In one embodiment, a pharmaceutical composition disclosed herein is formulated for controlled release or for slow release.

The pharmaceutical composition is administered, for example, by injection, including, but not limited to, subcutaneous, intravitreal, intradermal, intravenous, intra-arterial, intraperitoneal, intracerebrospinal, or intramuscular injection. Excipients and carriers for use in formulation of compositions for each type of injection are contemplated herein. The following descriptions are by example only and are not meant to limit the scope of the compositions. Compositions for injection include, but are not limited to, aqueous solutions (where water soluble) or dispersions, as well as sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In some embodiments, the carrier is a solvent or dispersion medium containing, for example, water, ethanol, polyol (for example, glycerol, propylene glycol, and liquid polyethylene glycol, and the like), and suitable mixtures thereof. Fluidity is maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid, and thimerosal. Isotonic agents, for example, sugars, polyalcohols such as mannitol, sorbitol, and sodium chloride is included in the composition in some embodiments. In some embodiments, the resulting solutions are packaged for use as is, or lyophilized; the lyophilized preparation is later be combined with a sterile solution prior to administration in some embodiments. For intravenous, injection, or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, and Lactated Ringer's Injection. Preservatives, stabilizers, buffers, antioxidants, and/or other additives are included, as needed, in some embodiments. Sterile injectable solutions are prepared by incorporating an active ingredient in the required amount in an appropriate solvent with one or a combination of ingredients enumerated above, as required, followed by filtered sterilization, in some embodiments. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

Compositions are conventionally administered intravenously in some embodiments, such as by injection of a unit dose, for example. For injection, in some embodiments, an active ingredient is in the form of a parenterally acceptable aqueous solution which is substantially pyrogen-free and has suitable pH, isotonicity, and stability. In some embodiments, one prepares suitable solutions using, for example, isotonic vehicles such as Sodium Chloride Injection, Ringer's Injection, Lactated Ringer's Injection. Preservatives, stabilizers, buffers, antioxidants, and/or other additives are included, as required, in some embodiments. Additionally, compositions are administered via aerosolization in some embodiments. (Lahn et al., Int Arch Allergy Immunol 134:49-55 (2004)).

For parenteral administration, the antibodies are formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate are also used. Liposomes are used as carriers. The vehicle contains minor amounts of additives such as substances that enhance isotonicity and chemical stability, e.g., buffers and preservatives. The antibodies are typically formulated in such vehicles at concentrations of about 1 mg/mL to 10 mg/mL.

In one embodiment, a pharmaceutical composition disclosed herein is lyophilized, for example, to increase shelf-life in storage. When the compositions are considered for use in medicaments or any of the methods provided herein, in some embodiments, it is contemplated that the composition are substantially free of pyrogens such that the composition will not cause an inflammatory reaction or an unsafe allergic reaction when administered to a human subject. Testing compositions for pyrogens and preparing compositions substantially free of pyrogens are well understood to one or ordinary skill of the art and are accomplished using commercially available kits in some embodiments.

In some embodiments, acceptable carriers contain a compound that stabilizes, increases or delays absorption or clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. In some embodiments, detergents also be used to stabilize or to increase or decrease the absorption of the pharmaceutical composition, including liposomal carriers. To protect from digestion the compound, in some embodiments, is complexed with a composition to render it resistant to acidic and enzymatic hydrolysis, or the compound is, in some embodiments, complexed in an appropriately resistant carrier such as a liposome. Means of protecting compounds from digestion are known in the art.

The compositions are administered, in some embodiments, in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable, but are typified by an initial administration followed by repeated doses at one or more hour intervals by a subsequent injection or other administration. Alternatively, continuous intravenous infusion that is sufficient to maintain concentrations in the blood are contemplated.

In some embodiments, the disclosure provides a use of the compositions described herein to make a medicament for treating a condition, disease, or disorder described herein. In some embodiments, medicaments are formulated based on the physical characteristics of the subject needing treatment, and are formulated in single or multiple formulations based on the stage of the condition, disease or disorder. Medicaments are packaged in a suitable package with appropriate labels for the distribution to hospitals and clinics in which the label is for the indication of treating a subject having a disease described herein in some embodiments. Medicaments are packaged as a single or multiple units in some embodiments. Instructions for the dosage and administration of the compositions are included with the packages as described below in some embodiments. The disclosure is further directed to medicaments comprising an antibody or antigen-binding fragment thereof described herein and a pharmaceutically acceptable carrier.

In some embodiments, amounts of the active ingredients in the compositions, the composition formulation, and the mode of administration, are among the factors that are varied to provide an amount of the active ingredient that is effective to achieve the desired therapeutic response for each subject, without being unduly toxic to the subject. The selected dosage level will depend upon a variety of factors including the activity of the particular compound employed, the route of administration, the time of administration, the rate of excretion or metabolism of the particular compound being employed, the duration of the treatment, other drugs, compounds and/or materials used in combination with the particular composition employed, the age, sex, weight, condition, general health, diet and prior medical history of the subject being treated, and like factors well known in the medical arts.

In some embodiments, the antibodies and antigen-binding fragments described herein are administered to a subject in various dosing amounts and over various time frames. Non-limiting doses include about 0.01 mg/kg, about 0.05 mg/kg, about 0.1 mg/kg, about 0.5 mg/kg, about 1 mg/kg, about 5 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or any integer in between. Additionally, the dose(s) of an antibody or antigen-binding fragment are administered, in some embodiments, twice a week, weekly, every two weeks, every three weeks, every 4 weeks, every 6 weeks, every 8 weeks, every 12 weeks, or any combination of weeks therein. Dosing cycles are also contemplated such as, for example, administering antibodies or antigen-binding fragments thereof once or twice a week for 4 weeks, followed by two weeks without therapy. Additional dosing cycles including, for example, different combinations of the doses and weekly cycles described herein are also contemplated within the disclosure.

Therapeutically effective amounts of a composition, in some embodiments, varies and depends on the severity of the disease and the weight and general state of the subject being treated, but generally range from about 1.0 pg/kg to about 100 mg/kg body weight, or about 10 pg/kg to about 30 mg/kg, or about 0.1 mg/kg to about 10 mg/kg or about 1 mg/kg to about 10 mg/kg per application. Administration can be daily, on alternating days, weekly, twice a month, monthly or more or less frequently, as necessary depending on the response to the disorder or condition and the subject's tolerance of the therapy. In some embodiments, maintenance dosages over a longer period of time, such as 4, 5, 6, 7, 8, 10, or 12 weeks or longer is needed until a desired suppression of disorder symptoms occurs, and dosages are adjusted as necessary. The progress of this therapy is easily monitored by conventional techniques and assays.

In some embodiments, the antibody of the disclosure is administered intravenously in a physiological solution at a dose ranging between 0.01 mg/kg to 100 mg/kg at a frequency ranging from daily to weekly to monthly (e.g., every day, every other day, every third day, or 2, 3, 4, 5, or 6 times per week), preferably a dose ranging from 0.1 to 45 mg/kg, 0.1 to 15 mg/kg or 0.1 to 10 mg/kg at a frequency of 2 or 3 times per week, or up to 45 mg/kg once a month.

A response is achieved when the subject experiences partial or total alleviation, or reduction of signs or symptoms of illness, and specifically includes, without limitation, prolongation of survival. The expected progression-free survival times are measured, for example, in months to years, depending on prognostic factors including the number of relapses, stage of disease, and other factors. Prolonging survival includes without limitation times of at least 1 month (mo), about at least 2 months (mos.), about at least 3 mos., about at least 4 mos., about at least 6 mos., about at least 1 year, about at least 2 years, about at least 3 years, or more. Overall survival is also be measured in months to years in some embodiments. The subject's symptoms remain static or decrease in some embodiments.

A physician or veterinarian having ordinary skill in the art, in some cases, readily determines and prescribes the effective amount (ED₅₀) of the composition required. For example, the physician or veterinarian could start doses of the compounds employed in the composition at levels lower than that required to achieve the desired therapeutic effect and gradually increase the dosage until the desired effect is achieved. Alternatively, a dose remains constant in some embodiments.

In some embodiments, a composition (an antibody or an antigen-binding fragment described herein) is administered alone or in combination with a second composition either simultaneously or sequentially dependent upon the condition to be treated. In one embodiment, a second therapeutic treatment is fibrosis therapy or fibrosis therapeutic. In one embodiment, a second therapeutic treatment is a treatment of a disease or disorder associated with fibrosis, such as an infection, an autoimmune disease or disorder, an inflammatory disease or disorder, or mechanical injury. When two or more compositions are administered, the compositions are, for example, administered in combination (either sequentially or simultaneously). A composition is administered in a single dose or multiple doses in some embodiments.

In some embodiments, when formulated for administration to human subjects, the compositions are formulated to be free of pyrogens. Testing compositions for pyrogens and preparing pharmaceutical compositions free of pyrogens are well understood to one of ordinary skill in the art.

Antibodies, or antigen-binding fragments thereof, are formulated for any suitable route of administration to a subject including, but not limited to injection, in some embodiments. Injection includes, for example, subcutaneous, peritoneal, intravenous injection, intramuscular injection, or spinal injection into the cerebrospinal fluid (CSF). In some embodiments, administration are in one, two, three, four, five, six, seven, or more injection sites. In one embodiment, administration is via six injection sites.

For in vivo applications, contacting occurs, for example, via administration of a composition (such as are described herein) to a subject by any suitable means. An antibody described herein, in some embodiments, is administered by any suitable means, either systemically or locally, including via parenteral, subcutaneous, intraperitoneal, intracerebrospinal, intrapulmonary, and intranasal administration, and, if desired for local treatment, intralesional administration. Parenteral routes include, for example, intravenous, intraarterial, intraperitoneal, epidural, intramuscular, and intrathecal administration. Such administration, in some embodiments, is as a bolus, continuous infusion, or pulse infusion. In some embodiments, compositions are administered by injection depending in part on whether the administration is brief or chronic. Other modes of administration methods are contemplated, including topical, particularly transdermal, transmucosal, rectal, oral or local administration e.g., through a catheter placed close to the desired site.

### Antibody Technology

As will be understood by the skilled artisan, general description of antibodies herein and methods of preparing and using the same also apply to individual antibody polypeptide constituents and antibody fragments.

The antibodies of the present disclosure are polyclonal or monoclonal antibodies. However, in preferred embodiments, they are monoclonal. In particular embodiments, antibodies of the present disclosure are human antibodies. Methods of producing polyclonal and monoclonal antibodies are known in the art.

Antibodies, antigen-binding fragments, and other proteins that bind huCD163 expressed by M2 macrophages are generated using such methods are tested for one or more of their binding affinity, avidity, and modulating capabilities in some embodiments.

Conventional methods, in some embodiments, are utilized to identify antibodies or antigen-binding fragments thereof that bind to a huCD163 protein. In some embodiments, antibodies and antigen-binding fragments are evaluated for one or more of binding affinity, association rates, disassociation rates, and avidity. Measurement of such parameters is, for example, accomplished using assays including, but not limited to, an enzyme-linked-immunosorbent assays (ELISA), ELISpot assays, Scatchard analysis, surface plasmon resonance (e.g., BIACORE) analysis, etc., competitive binding assays, and the like. In one non-limiting embodiment, an ELISA assay is used to measure the binding capability of specific antibodies or antigen-binding fragments that bind to a huCD163 protein. A surface plasmon resonance technique is described in Liljeblad et al., Glyco J 17:323-9 (2000).

In some embodiments, antibodies according to the disclosure are produced recombinantly, using vectors and methods available in the art, as described further below. In some embodiments, human antibodies are also be generated by in vitro activated B cells (see U.S. Pat. Nos. 5,567,610 and 5,229,275).

In some embodiments, human antibodies are produced in transgenic animals (e.g., mice) that are capable of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice results in the production of human antibodies upon antigen challenge. See, e.g., Jakobovits et al., Proc Natl Acad Sci USA, 90:2551 (1993); Jakobovits et al., Nature 362:255-58 (1993); Bruggemann et al., Year in Immunol., 7:33 (1993); U.S. Pat. Nos. 5,545,806, 5,569,825, 5,591,669; 5,545,807; and WO 97/17852. In some embodiments, such animals are genetically engineered to produce human antibodies comprising a polypeptide of the present disclosure.

The antibodies are, for example, isolated and purified from a culture supernatant or ascites (if produced in an animal) using methods known in the art, such as by saturated ammonium sulfate precipitation, euglobulin precipitation method, caproic acid method, caprylic acid method, ion exchange chromatography (DEAE or DE52), or affinity chromatography using anti-Ig column or a protein A, G, or L column.

As noted above, the disclosure further provides antibody fragments. In certain circumstances there are advantages of using antibody fragments, rather than whole antibodies. For example, the smaller size of the fragments allows for rapid clearance, and leads to improved access to certain tissues, such as organs (e.g., lung, kidney, liver, or heart), in some embodiments. Examples of antibody fragments include: Fab, F(ab′), F(ab′)₂, and Fv fragments, diabodies: linear antibodies; single-chain antibodies; and multispecific antibodies formed from antibody fragments.

Various techniques have been developed to produce antibody fragments. Traditionally, these fragments were derived via proteolytic digestion of intact antibodies (see, e.g., Morimoto et al., J Biochem Biophys Methods 1992 24(1-2):107-17; and Brennan et al., Science 1985 229:81).

However, these fragments are now be produced directly by recombinant host cells in some embodiments. In some embodiments, Fab, Fv, and ScFv antibody fragments all are expressed in and secreted from E coli, thus allowing the facile production of large amounts of these fragments. In some embodiments, F(ab′)-SH fragments are directly recovered from E. coli and chemically coupled to form F(ab′)₂ fragments (Carter et al., Bio/Technology 10:163-167 (1992)). According to another approach, in some embodiments. F(ab′)₂ fragments are isolated directly from recombinant host cell culture. Fab and F(ab′)₂ fragment with increased in vivo half-life comprising a salvage receptor binding epitope taken from two loops of a C_{H}2 domain of an Fc region of an IgG are described in U.S. Pat. Nos. 5,869,046 and 6,121,022. Other techniques for producing antibody fragments will be apparent to the skilled practitioner.

In other embodiments, the antibody of choice is a single chain Fv fragment (scFv). See WO 93/16185; U.S. Pat. Nos. 5,571,894; and 5,587,458. Fv and sFv are the only species with intact combining sites that are devoid of constant regions. Thus, they are suitable for reduced nonspecific binding during in vivo use. In some embodiments, sFv fusion proteins are constructed to yield fusion of an effector protein at either the amino or the carboxy terminus of an sFv. See Antibody Engineering, ed. Borrebaeck, supra. In some embodiments, the antibody fragment is a “linear antibody,” e.g., as described in U.S. Pat. No. 5,641,870 for example. In some embodiments, such linear antibody fragments are monospecific or bispecific.

Methods for making bispecific or other multispecific antibodies are known in the art and include chemical cross-linking, use of leucine zippers (Kostelny et al., J Imnunol 148:1547-53 (1992)); diabody technology (Hollinger et al., Proc Natl Acad Sci USA 90:6444-8 (1993)); scFv dimers (Gruber et al., J Immunol 152:5368 (1994)), linear antibodies (Zapata et al., Protein Eng 8:1057-62 (1995)); and chelating recombinant antibodies (Neri et al., J Mol Biol 246:367-73 (1995)).

Traditional production of full-length bispecific antibodies is based on the co-expression of two immunoglobulin heavy chain-light chain pairs, where the two chains have different specificities (Millstein et al., Nature 305:537-9 (1983)). Because of the random assortment of immunoglobulin heavy and light chains, these hybridomas (quadromas) produce a potential mixture of ten different antibody molecules, of which only one has the correct bispecific structure. Purification of the correct molecule are done, for example, by affinity chromatography. Similar procedures are disclosed in WO 93/08829, and in Traunecker et al., EMBO J 10:3655-9 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred that the first heavy-chain constant region (C_{H}1) containing the site necessary for light chain bonding, be present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the four polypeptide fragments in embodiments when unequal ratios of the four polypeptide chains used in the construction provide the optimum yield of the desired bispecific antibody. It is, however, possible to insert the coding sequences for two or all four polypeptide chains into a single expression vector w % ben the expression of at least two polypeptide chains in equal ratios results in high yields or when the ratios have no significant effect on the yield of the desired chain combination.

Bispecific antibodies are composed of, for example, a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods Enzymol 121:210 (1986).

According to another approach described in U.S. Pat. No. 5,731,168, the interface between a pair of antibody molecules are engineered to maximize the percentage of heterodimers that are recovered from recombinant cell culture in some embodiments. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate are coupled to avidin, the other to biotin. Such antibodies have, for example, been proposed to target immune system cells to unwanted cells (U.S. Pat. No. 4,676,980), and for treatment of HIV infection (WO 91/00360, WO 92/20373, and EP 03089). In some embodiments, heteroconjugate antibodies are made using any convenient cross-linking methods. Suitable cross-linking agents are well known in the art, and are disclosed in U.S. Pat. No. 4,676,980, along with a number of cross-linking techniques. Another method is designed to make tetramers by adding a streptavidin-coding sequence at the C-terminus of the scFv. Streptavidin is composed of four subunits, so when the scFv-streptavidin is folded, four subunits associate to form a tetramer (Kipriyanov et al., Hum Antibodies Hybridomas 6(3):93-101 (1995)).

According to another approach for making bispecific antibodies, the interface between a pair of antibody molecules are engineered to maximize the percentage of heterodimers which are recovered from recombinant cell culture in some embodiments. One interface comprises at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. See WO 96/27011.

Techniques for generating bispecific antibodies from antibody fragments have also been described in the literature. For example, bispecific antibodies are prepared using chemical linkage. Brennan et al., Science 229; 81 (1985) describes a procedure wherein intact antibodies are proteolytically cleaved to generate F(ab′)₂ fragments. These fragments are reduced in the presence of the dithiol complexing agent, sodium arsenite, to stabilize vicinal dithiols, and prevent intermolecular disulfide formation. The F(ab′) fragments generated are then converted to thionitrobenzoate (TNB) derivatives. One of the F(ab′)-TNB derivatives is then reconverted to the F(ab′)-thiol by reduction with mercaptoethylamine and is mixed with an equimolar amount of the other F(ab′)-TNB derivative to form the bispecific antibody. In some embodiments, the bispecific antibodies produced are used as agents for the selective immobilization of enzymes.

Recent progress has facilitated the direct recovery of F(ab′)-SH fragments from E. coli, which are, for example, chemically coupled to form bispecific antibodies. Shalaby et al., J Exp Med 175: 217-25 (1992) describes the production of a humanized bispecific antibody F(ab′)₂ molecule. Each F(ab′) fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J Immunol 148(5):1547-53 (1992). The leucine zipper peptides from the Fos and Jun proteins were linked to the F(ab′) portions of two different antibodies by gene fusion. The antibody homodimers were reduced at the hinge region to form monomers and then re-oxidized to form the antibody heterodimers. This method is be used to produce antibody homodimers in some embodiments.

### Identification and Preparation of Antibodies

Polynucleotide sequences encoding the antibodies, variable regions thereof, or antigen-binding fragments thereof are, in some embodiments, determined using conventional sequencing techniques, and subcloned into expression vectors for the recombinant production of the antibodies. This was accomplished by obtaining mononuclear cells from the blood of a subject; producing B cell clones from the mononuclear cells; inducing the B cells to become antibody-producing plasma cells; and screening the supernatants produced by the plasma cells to determine if it contains an antibody. Identification of other antibodies having the specificity of the antibodies of the disclosure are accomplished using a similar method in some embodiments. For example, once a B cell clone that produces an antibody is identified, reverse-transcription polymerase chain reaction (RT-PCR) is performed to clone the DNAs encoding the variable regions or portions thereof of the antibody. These sequences are then subcloned into expression vectors suitable for the recombinant production of human antibodies. The binding specificity is confirmed, in some embodiments, by determining the antibody's ability to bind M2 cells or other cells expressing a human CD163 polypeptide that is expressed by M2 cells.

In particular embodiments of the methods described herein, B cells isolated from peripheral blood or lymph nodes are sorted, e.g., based on their being CD19 positive, and plated, e.g., as low as a single cell specificity per well, e.g., in 96-, 384-, or 1536-well configurations. The cells are induced to differentiate into antibody-producing cells, e.g., plasma cells, and the culture supernatants are harvested and tested for binding to cells expressing the target polypeptide on their surface using, e.g., FMAT or FACS analysis. Positive wells are then subjected to w % bole well RT-PCR to amplify heavy and light chain variable regions of the IgG molecule expressed by the clonal daughter plasma cells. The resulting PCR products encoding the heavy and light chain variable regions, or portions thereof, are subcloned into human antibody expression vectors for recombinant expression. The resulting recombinant antibodies are then tested to confirm their original binding specificity and are further tested, in some embodiments, for cross-reactivity against other cells or proteins.

Thus, in one embodiment, a method of identifying antibodies is practiced as follows. First, full-length or approximately full-length CD163 cDNAs are transfected into a cell line for expression of CD163 polypeptides. Secondly, individual human plasma or sera samples are tested for antibodies that bind the cell-expressed polypeptides. And lastly, MAbs derived from plasma- or serum-positive individuals are characterized for binding to the same cell-expressed CD163 polypeptides. Further definition of the fine specificities of the MAbs are performed at this point in some embodiments.

Polynucleotides that encode the antibodies or portions thereof of the present disclosure are isolated from cells expressing the antibodies, according to methods available in the art and described herein, including amplification by polymerase chain reaction using primers specific for conserved regions of human antibody polypeptides, in some embodiments. For example, light chain and heavy chain variable regions is cloned from the B cell according to molecular biology techniques described in WO 92/02551; U.S. Pat. No. 5,627,052; or Babcook et al., Proc Natl Acad Sci USA 93.7843-48 (1996). In certain embodiments, polynucleotides encoding all or a region of both the heavy and light chain variable regions of the IgG molecule expressed by the clonal daughter plasma cells expressing the antibody are subcloned and sequenced. In some embodiments, the sequence of the encoded polypeptide is readily determined from the polynucleotide sequence.

Isolated polynucleotides encoding a polypeptide of the present disclosure is subcloned into an expression vector to recombinantly produce antibodies and polypeptides of the present disclosure, using procedures known in the art and described herein.

In some embodiments, binding properties of an antibody (or fragment thereof) to CD163 polypeptides or M2 cells are generally determined and assessed using immunodetection methods including, for example, immunofluorescence-based assays, such as immuno-histochemistry (IHC) and/or fluorescence-activated cell sorting (FACS). Immunoassay methods include, in some embodiments, controls and procedures to determine whether antibodies bind specifically to CD163 polypeptides or to M2 macrophages, and do not recognize or cross-react with control cells, e.g., M1 cells, or host cells transfected to express a control protein.

Following pre-screening of serum to identify patients that produce antibodies to a CD163 polypeptide or to M2 macrophages, the methods of the present disclosure typically include the isolation or purification of B cells from a biological sample previously obtained from a patient or subject. In some embodiments, the patient or subject are currently or have previously been diagnosed with or suspect or having fibrosis, or the patient or subject is considered free of fibrosis. Typically, the patient or subject is a mammal and, in particular embodiments, a human. In some embodiments, the biological sample is any sample that contains B cells, including but not limited to, fibrotic tissue, lymph node or lymph node tissue, pleural effusions, peripheral blood, ascites, or cerebrospinal fluid (CSF). In some embodiments, B cells are isolated from different types of biological samples, such as a biopsy of fibrotic tissue or other biological sample affected by fibrosis. However, in some embodiments, it is understood that any biological sample comprising B cells is used for any of the embodiments of the present disclosure.

Once isolated, the B cells are induced to produce antibodies, e.g., by culturing the B cells under conditions that support B cell proliferation or development into a plasmacyte, plasmablast, or plasma cell. The antibodies are then screened, typically using high throughput techniques, to identify an antibody that specifically binds to a target antigen, e.g., a particular tissue, cell, or polypeptide. In certain embodiments, the specific antigen, e.g., cell surface polypeptide bound by the antibody is not known, while in other embodiments, the antigen specifically bound by the antibody is known.

According to the present disclosure, B cells are, in some embodiments, isolated from a biological sample, e.g., tissue, peripheral blood or lymph node sample, or fibrotic tissue by any means known and available in the art. B cells are typically sorted by FACS based on the presence on their surface of a B cell-specific marker, e.g., CD19, CD138, and/or surface IgG. However, other methods known in the art are employed in some embodiments, such as, e.g., column purification using CD19 magnetic beads or IgG-specific magnetic beads, followed by elution from the column. However, magnetic isolation of B cells utilizing any marker results in loss of certain B cells in some embodiments. Therefore, in certain embodiments, the isolated cells are not sorted but, instead, Ficoll-purified mononuclear cells isolated from fibrotic tissue that are directly plated to the appropriate or desired number of specificities per well.

To identify B cells that produce an antibody, the B cells are typically plated at low density (e.g., a single cell specificity per well, 1-10 cells per well, 10-100 cells per well, 1-100 cells per well, less than 10 cells per well, or less than 100 cells per well) in multi-well or microtiter plates. e.g., in 96, 384, or 1536 well configurations. When the B cells are initially plated at a density greater than one cell per well, then the methods of the present disclosure include the step of subsequently diluting cells in a well identified as producing an antigen-specific antibody, until a single cell specificity per well is achieved, thereby facilitating the identification of the B cell that produces the antigen-specific antibody in some embodiments. In some embodiments, cell supernatants or a portion thereof and/or cells are frozen and stored for future testing and later recovery of antibody polynucleotides.

In certain embodiments, the B cells are cultured under conditions that favor the production of antibodies by the B cells. For example, the B cells are cultured under conditions favorable for B cell proliferation and differentiation to yield antibody-producing plasmablasts, plasmacytes, or plasma cells. In particular embodiments, the B cells are cultured in the presence of a B cell mitogen, such as lipopolysaccharide (LPS) or CD40 ligand. In one specific embodiment. B cells are differentiated to antibody-producing cells by culturing them with feed cells and/or other B cell activators, such as CD40 ligand.

Cell culture supernatants or antibodies obtained therefrom are tested for their ability to bind to a target antigen, using routine methods available in the art, including those described herein, in some embodiments. In particular embodiments, culture supernatants are tested for the presence of antibodies that bind to a target antigen using high-throughput methods. For example, B cells are cultured in multi-well microtiter dishes, such that robotic plate handlers are used to simultaneously sample multiple cell supernatants and test for the presence of antibodies that bind to a target antigen. In particular embodiments, antigens are bound to beads, e.g., paramagnetic or latex beads) to facilitate the capture of antibody/antigen complexes. In other embodiments, antigens and antibodies are fluorescently labeled (with different labels) and FACS analysis is performed to identify the presence of antibodies that bind to target antigen. In one embodiment, antibody binding is determined using FMAT™ analysis and instrumentation (Applied Biosystems, Foster City, Calif.). FMAT is a fluorescence macro-confocal platform for high-throughput screening, which enables mix-and-read, non-radioactive assays using live cells or beads.

In comparing the binding of an antibody to a particular target antigen (e.g., a biological sample such as diseased tissue or cells, fibrotic tissue or cells, or infectious agents) to the antibody's binding to a control sample (e.g., a biological sample such as normal cells, comparator cells from another species, a different fibrotic tissue or cell, a different tissue or cell, or different infectious agent), in some embodiments, the antibody is considered to preferentially bind a particular target antigen if at least two-fold, at least three-fold, at least five-fold, or at least ten-fold more antibody binds to the particular target antigen as compared to the amount that binds a control sample.

Polynucleotides encoding antibody chains, variable regions thereof, or fragments thereof, are isolated from cells utilizing any means available in the art in some embodiments. In one embodiment, polynucleotides are isolated using polymerase chain reaction (PCR), e.g., reverse transcription-PCR (RT-PCR) using oligonucleotide primers that specifically bind to heavy or light chain encoding polynucleotide sequences or complements thereof using routine procedures available in the art. In one embodiment, positive wells are subjected to whole well RT-PCR to amplify the heavy and light chain variable regions of the IgG molecule expressed by the clonal daughter plasma cells. These PCR products, in some embodiments, are sequenced, and products encoding the heavy and light chain variable regions or portions thereof are then subcloned into human antibody expression vectors and recombinantly expressed according to routine procedures in the art (see, e.g., U.S. Pat. No. 7,112,439). The nucleic acid molecules encoding a M2 macrophage-specific antibody or fragment thereof as described herein are, in some embodiments, propagated and expressed according to any of a variety of well-known procedures for nucleic acid excision, ligation, transformation, and transfection. Thus, in certain embodiments expression of an antibody fragment are preferred in a prokaryotic host cell, such as E. coli (see, e.g., Pluckthun et al., Methods Enzymol 178:497-515 (1989)). In certain other embodiments, expression of the antibody or an antigen-binding fragment thereof are preferred in a eukaryotic host cell, such as yeast (e.g., Saccharomyces cerevistae, S. pombe, Pichia pastoris); animal cells (including mammalian cells); or plant cells. Examples of suitable animal cells include, but are not limited to, myeloma, COS, CHO, or hybridoma cells. Examples of plant cells include tobacco, corn, soybean, and rice cells. By methods known to those having ordinary skill in the art and based on the present disclosure, a nucleic acid vector is designed for expressing foreign sequences in a particular host system, and then polynucleotide sequences encoding the M2 macrophage-specific antibody (or fragment thereof) is inserted, in some embodiments. The regulatory elements will vary according to the particular host.

One or more replicable expression vectors containing a polynucleotide encoding a variable and/or constant region is, in some embodiments, prepared and used to transform an appropriate cell line, for example, a non-producing myeloma cell line, such as a mouse NSO line or a bacterium, such as E. coli, in which production of the antibody will occur. In order to obtain efficient transcription and translation, the polynucleotide sequence in each vector should include appropriate regulatory sequences, particularly a promoter and leader sequence operatively linked to the variable region sequence.

Particular methods for producing antibodies in this way are generally well known and routinely used. For example, molecular biology procedures are described by Sambrook et al., Molecular Cloning, A Laboratory Manual, 2nd ed. Cold Spring Harbor Laboratory, New York, 1989; see also Sambrook et al., 3rd ed., Cold Spring Harbor Laboratory, New York, (2001)). While not required, in certain embodiments, regions of polynucleotides encoding the recombinant antibodies are sequenced. DNA sequencing are performed, for example, in any manner or using any systems known in the art. Basic sequencing technology is described for example, in Sanger et al., Proc Natl Acad Sci USA 74:5463 (1977)) and the Amersham International plc sequencing handbook and including improvements thereto.

In particular embodiments, the resulting recombinant antibodies or fragments thereof are then tested to confirm their original specificity, and are further tested for cross-reactivity, e.g., with related polypeptides, in some embodiments. In particular embodiments, an antibody identified or produced according to methods described herein is tested for ability to internalize or other effector function using conventional methods.

### Packages, Kits, and Pre-Filled Containers

Also provided herein are kits containing one or more compounds described above. The kit comprises, in some embodiments, an antibody or antigen-binding fragment thereof as described herein in suitable container means.

In some embodiments, there is provided is a container means comprising a composition described herein. In some embodiments, the container means is any suitable container which houses, for example, a liquid or lyophilized composition including, but not limited to, a vial, syringe, bottle, an in intravenous (IV) bag or ampoule. A syringe holds any volume of liquid suitable for injection into a subject, in some embodiments, including, but not limited to, 0.5 cc, 1 cc, 2 cc, 5 cc, 10 cc, or more.

Provided herein are kits, comprising a composition or compositions described herein. In some embodiments, provided herein is a kit for treating a subject having a fibrosis, comprising an antibody as described herein and a fibrosis therapy.

In some embodiments, provided herein is a kit for treating a fibrosis, comprising an antibody as described herein, and a label attached to or packaged with the container, the label describing use of the antibody in combination with a fibrosis therapy.

In some embodiments, provided herein is a kit for treating a fibrosis, comprising a fibrosis therapy and a label attached to or packaged with the container, the label describing use of the fibrosis therapy (e.g., an anti-inflammatory) with an antibody as described herein.

In some embodiments, the container means of the kits will generally include at least one vial, test tube, flask, bottle, ampoule, syringe an intravenous (IV) bag, and/or other container means, into which the at least one polypeptide are placed, and/or preferably, suitably aliquoted. Provided herein is a container means comprising a composition described herein.

The kits, in some embodiments, include a means for containing at least one fusion protein, detectable moiety, reporter molecule, and/or any other reagent containers in close confinement for commercial sale. In some embodiments, such containers include injection and/or blow-molded plastic containers into which the desired vials are retained. In some embodiments, kits also include printed material for use of the materials in the kit.

Packages and kits additionally include a buffering agent, a preservative, and/or a stabilizing agent in a pharmaceutical formulation in some embodiments. In some embodiments, each component of the kit is enclosed within an individual container and all of the various containers can be within a single package. In some embodiments, disclosure kits are designed for cold storage or room temperature storage.

Additionally, in some embodiments, the preparations contain stabilizers to increase the shelf-life of the kits and include, for example, bovine serum albumin (BSA). Where the compositions are lyophilized, the kit contains, in some embodiments, further preparations of solutions to reconstitute the lyophilized preparations. Acceptable reconstitution solutions are well known in the art and include, for example, pharmaceutically acceptable phosphate buffered saline (PBS).

In some embodiments, packages and kits further include one or more components for an assay, such as, for example, an ELISA assay. Samples to be tested in this application include, for example, blood, plasma, tissue sections and secretions, urine, lymph, and products thereof. In some embodiments, packages and kits further include one or more components for collection of a sample (e.g., a syringe, a cup, a swab, etc.).

In some embodiments, packages and kits further include a label specifying information required by US FDA or similar regulatory authority, for example, a product description, amount and mode of administration, and/or indication of treatment. Packages provided herein can include any of the compositions as described herein.

The term “packaging material” refers to a physical structure housing the components of the kit. In some embodiments, the packaging material maintains the components sterilely and are made of material commonly used for such purposes (e.g., paper, corrugated fiber, glass, plastic, foil, ampules, etc.). In some embodiments, the label or packaging insert includes appropriate written instructions. Kits, therefore, additionally includes, in some embodiments, labels or instructions for using the kit components in any method of the disclosure. In some embodiments, a kit includes a compound in a pack, or dispenser together with instructions for administering the compound in a method described herein.

In still further embodiments, a kit further comprises a container means for fibrosis therapy.

Instructions include instructions for practicing any of the methods described herein including treatment methods in some embodiments. Instructions additionally include indications of a satisfactory clinical endpoint or any adverse symptoms that occur, or additional information required by regulatory agencies such as the Food and Drug Administration for use on a human subject in some embodiments.

The instructions are, in some embodiments, on “printed matter,” e.g., on paper or cardboard within or affixed to the kit, or on a label affixed to the kit or packaging material, or attached to a vial or tube containing a component of the kit. Instructions are additionally included on a computer readable medium, such as, for example, CD-ROMs, DVDs, flash memory devices, solid state memory, magnetic disks and disk devices, magnetic tapes, cloud computing systems and services, and the like, in some embodiments. In some cases, the program and instructions are permanently, substantially permanently, semi-permanently, or non-transitorily encoded on the media.

Provided herein is a container means comprising a composition described herein. In some embodiments, the container means is any suitable container which houses a liquid or lyophilized composition including, but not limited to, a vial, syringe, bottle, intravenous (IV) bag, or ampoule. A syringe, in some embodiments, holds any volume of liquid suitable for injection into a subject including, but not limited to, 0.5 cc, 1 cc, 2 cc, 5 cc, 10 cc or more.

Provided herein are kits comprising a composition described herein. In some embodiments, provided herein is a kit for treating fibrosis, comprising an antibody as described herein in combination with a fibrosis therapy agent.

In some embodiments, provided herein is a kit for treating fibrosis, such as fibrosis associated with a presence of M2-macrophages, comprising an antibody as described herein, and a label attached to or packaged with the container, the label describing use of the antibody, or an antigen-binding fragment thereof, with an additional anti-fibrosis therapy or anti-inflammatory therapy. In some embodiments, the fibrosis is a primary fibrotic disease. In some embodiments, the fibrosis is a secondary fibrotic disease.

In some embodiments, provided herein is a kit for treating fibrosis, such as fibrosis associated with a presence of M2-macrophages, comprising an additional anti-fibrosis therapy or anti-inflammatory therapy and a label attached to or packaged with the container, the label describing use of the additional anti-fibrosis therapy or anti-inflammatory therapy with an antibody as described herein. In some embodiments, the fibrosis is a primary fibrotic disease. In some embodiments, the fibrosis is a secondary fibrotic disease.

### EXAMPLES

The present disclosure will be further illustrated in the following Examples which are given for illustration purposes only and are not intended to limit the disclosure in any way.

### Example 1—Affinity Maturation

The results of phage ELISA showed that AB101-scFv-HL can be displayed at the N-terminal of pill protein at the tail of the phage through the E. coli TG1/pCantab 5E phagemid display system. The EC₅₀ value of AB101-E and AB101-C binding to huCD163 was 4.34 nM and 5.44 nM, respectively.

V_{H} and V_{L} mutated phage display scFv libraries were designed and constructed via error-prone PCR (marked as scFv-AB101 VHmu-VLwt library and VHwt-VLmu library).

scFv-AB101 VHmu-VLwt Library

| Library | | Volume of | | Dilution | No. of |
|---|---|---|---|---|---|
| Name | Capacity | transformation | Round | ratio | clones |
| scFv-AB101 | 1.13 × 10¹⁰ | 24 mL | 1^{st} | 10⁴ | N/A |
| VHmu-VLwt | | | | 10⁵ | 1200 |
| library | | | | 10⁶ | 169 |
| | | 24 mL | 2^{nd} | 10⁴ | N/A |
| | | | | 10⁵ | 1200 |
| | | | | 10⁶ | 174 |
| | | 24 mL | 3^{rd} | 10⁴ | N/A |
| | | | | 10⁵ | 1200 |
| | | | | 10⁶ | 189 |

scFv-AB101 VHwt-VLmu Library′

| Library | | Volume of | | Dilution | No. of |
|---|---|---|---|---|---|
| Name | Capacity | transformation | Round | ratio | clones |
| scFv-AB101 | 1.03 × 10¹⁰ | 26 mL | 1^{st} | 10⁴ | N/A |
| VHwt-VLmu | | | | 10⁵ | 996 |
| library | | | | 10⁶ | 138 |
| | | 24 mL | 2^{nd} | 10⁴ | N/A |
| | | | | 10⁵ | 1100 |
| | | | | 10⁶ | 141 |
| | | 24 mL | 3^{rd} | 10⁴ | N/A |
| | | | | 10⁵ | 1200 |
| | | | | 10⁶ | 179 |

According to the sequence alignment analysis based on the antibody database and structural modeling analysis of Discovers' Studio, a Hotspot mutation phage display scFv library was designed and constructed via NNK Primers (marked as scFv-AB101 Hotspot mutation library).

scFv-AB101 Hotspot Mutation Library

| Library | | Volume of | | Dilution | No. of |
|---|---|---|---|---|---|
| Name | Capacity | transformation | Round | ratio | clones |
| scFv-AB101 | 1.0 × 10⁹ | 10 mL | 1^{st} | 10⁴ | N/A |
| Hotspot | | | | 10⁵ | 852 |
| mutation | | | | 10⁶ | 106 |
| library | | | | | |

The capacity of the three mutant libraries was 1.13×10¹⁰ (VHmu-VLwt library), 1.03×10¹⁰ (VHwt-VLmu library), and 1.0×10⁹ (Hotspot mutation library) respectively. The quality of each library was tested by sequencing with S1 primer. The correction rate of each mutant library is 77.8% (VHmu-VLwt library), 70% (VHwt-VLmu library), and 75% (Hotspot mutation library). These results indicated that three libraries were constructed with high quality.

Library screenings were performed on the three mutant libraries, marked as scFv-AB101 VHmu-VLwt library, VHwt-VLmu library and Hotspot mutation library.

A SDS-PAGE analysis of target antigen, human CD163 was performed. The molecular weight of human CD163 was about 140 K_{D}, the purity was above 90% without any degradation. Biotin-labeled human CD163 (marked as b-huCD163) was generated, and a QC test was conducted. In detail, SDS-PAGE associated with Streptavidin was used to evaluate the biotin labeling efficiency, and the result showed the efficiency was above 95%. All the results indicated that the quality of human CD163 and b-huCD163 were of a high level.

Four rounds of library screening were performed based on the three mutant libraries against b-huCD163. Enriching effects were found for all the three libraries, i.e., scFv-AB101 VHmu-VLwt library, VHwt-VLmu library and Hotspot mutation library.

### VHmu-VLwt Library

138 clones were randomly selected from the 4th round output of VHmu-VLwt library screening and placed in 96-well plates (marked as 1-46, 137-182, and 273-318) for QC monoclonal phage ELISA using AB101 parental phage as a control. According to phage ELISA results, 40 highly positive clones were selected from Batch 1-3 for DNA sequencing (clone 178 and 181 failed to be sequenced).

VHwt-VLmu Library

138 clones were randomly selected from the 4th round output of VHwt-VLmu library screening and placed in 96-well plates (marked as 47-92, 183-228, and 319-364) for QC monoclonal phage ELISA using AB101 parental phage as a control. According to phage ELISA results, 42 highly positive clones were selected from Batch 1-3 for DNA sequencing.

Hotspot Mutation Library

138 clones were randomly selected from the 4th round output of Hotspot mutation library screening and placed in 96-well plates (marked as 93-136, 229-272, 365-408) for QC monoclonal phage ELISA using AB101 parental phage as a control. According to phage ELISA results, 17 highly positive clones were selected from Batch 1-3 for DNA sequencing. In total, 97 highly positive clones were selected for DNA sequencing.

Sub-Library Construction and Screening

The VHm (containing mutations in VH) sequences from the 4th round output pool of VHm-VLwt library, and VLm (containing mutations in VL) sequences from the 4th round output pool of VHwt-VLm library were amplified, respectively, and randomly assembled to scFvs via overlap PCR to construct a sub-library. The end sub-library was constructed, and the capacity of the library is 3.8×10⁹. Two rounds of library screening were performed based on the sub-library against the antigen. After that, 180 clones (marked as clone 409-588) were randomly selected out from the 2nd round output of the sub-library to perform monoclonal phage ELISA. Based on the monoclonal phage ELISA results, 60 clones with the highest positive signal were selected out for sequencing. According to the DNA sequencing result, 56 clones have been sequenced successfully and 4 clones (clone 411, 462, 520, and 556) failed to obtain the correct sequence. Based on the characteristic of sequence, 5 sequences were selected for further IgG antibody eukaryotic expression and affinity evaluation.

### Example 2—Determination of Antibody Binding Affinity

BLI affinity detection was performed with one Ab concentration for the 6 clones by using AB101 parental Ab as a control. All clones had lower K_{D} value, indicating they had enhanced affinity when compared to AB101 parental Ab.

Materials

Antigen: Human CD163 Protein (biotinylated)

The following antibodies were tested: Variant 1 (V1)-Variant 6 (V6). The molecular weight (MW) and concentrations are as described in Table 4:

TABLE 4

| Antibody | M.W. (KDa) | Concentration |
|---|---|---|
| AB101 parental Ab | 149 | 1.2 mg/mL |
| Variant 1 (V1) | 149 | 2.3 mg/mL |
| Variant 2 (V2) | 149 | 2.2 mg/mL |
| Variant 3 (V3) | 149 | 1.4 mg/mL |
| Variant 4 (V4) | 149 | 1.2 mg/mL |
| Variant 5 (V5) | 149 | 1 mg/mL |
| Variant 6 (V6) | 149 | 1.2 mg/mL |

Buffer: PBS, PBST

Equipment: ForteBio Octet RED96 (Pall), SA Biosensor

Protocol

Samples were prepared as follows; Dilute antigen b-huCD163 to 2 pg/mL with PBS buffer; Dilute AB101 parental Ab to a series of concentration (100/150/200/250 nM) with PBST buffer. Results are described in Table 5:

TABLE 5

| | | | | Ab |
|---|---|---|---|---|
| Clone | K_{D} | Ka | Kd | concentration |
| name | (M) | (M⁻¹ · s⁻¹) | (s⁻¹) | ( nM) |
| AB101 | 8.78 × 10⁻⁷ | 3.37 × 10⁴ | 2.96 × 10⁻² | 50.0 nM |
| parental | | | | |
| Ab | | | | |
| V1 | 1.25 × 10⁻⁹ | 8.95 × 10⁵ | 1.12 × 10⁻³ | 50.0 nM |
| V2 | 9.91 × 10⁻¹⁰ | 8.78 × 10⁵ | 8.70 × 10⁻⁴ | 50.0 nM |
| V3 | 9.37 × 10⁻¹⁰ | 9.87 × 10⁵ | 9.25 × 10⁻⁴ | 50.0 nM |
| V4 | 8.24 × 10⁻¹⁰ | 1.06 × 10⁶ | 8.74 × 10⁻⁴ | 50.0 nM |
| V5 | 1.03 × 10⁻⁹ | 9.51 × 10⁵ | 9.78 × 10⁻⁴ | 50.0 nM |
| V6 | 9.64 × 10⁻¹⁰ | 9.21 × 10⁵ | 8.87 × 10⁻⁴ | 50.0 nM |

V4 was selected out for BLI affinity measurement with 4 Ab concentrations. Dilute V4 antibody to a series of concentration (6.25/12.5/25/50 nM) with PBST buffer. Results are described in Table 6:

TABLE 6

| | | | | Ab | Affinity enhancement |
|---|---|---|---|---|---|
| Clone | K_{D} | Ka | Kd | concentration | compared to parental |
| name | (M) | (M⁻¹ · s⁻¹) | (s⁻¹) | (nM) | Ab |
| AB101 | 6.61 × 10⁻⁷ | 4.78 × 10⁴ | 3.16 × 10⁻² | 100/150/200/250 | N/A |
| parental Ab | | | | | |
| V4 | 1.76 × 10⁻¹⁰ | 2.23 × 10⁶ | 3.93 × 10⁻⁴ | 6.25/12.5/25/50 | 3755-fold |

Affinity was determined as follows:

| Step | Description | Solution | Time(s) |
|---|---|---|---|
| 1 | Equilibrium | PBS buffer | 180 |
| 2 | Immobilization | 2 μg/mL huCD163 | 120 |
| 3 | Equilibrium | PBST buffer | 300 |
| 4 | Association | Different concentration of Ab solution | 120 |
| 5 | Dissociation | PBST buffer | 480 |

The SA sensors were placed in the PBS buffer for 15 minutes. Different solutions were added to a 96-well plate according to the following orders, with 200 μL per well.

| | | | |
|---|---|---|---|
| PBS buffer | 2 μg/mL b-huCD163 | PBST buffer | Ab solution |
| PBS buffer | 2 μg/mL b-huCD163 | PBST buffer | Ab solution |
| PBS buffer | 2 μg/mL b-huCD163 | PBST buffer | Ab solution |
| PBS buffer | 2 μg/mL b-huCD163 | PBST buffer | Ab solution |
| PBS buffer | 2 μg/mL b-huCD163 | PBST buffer | PBST buffer |

It is notable that the clone 4 had a K_{D} of 1.76×10¹⁰, which was about 3755-fold affinity enhancement when compared to AB101 parental Ab with a K_{D} value of 6.61×10⁻⁷.

### Example 3—Treatment of Lung Carcinoma

Subjects diagnosed or suspected of having a lung carcinoma are administered one or more therapeutically effective doses of an antibody disclosed herein. The lung carcinoma is ameliorated or eliminated following treatment.

### Example 4—Treatment of Lung Sarcoma

Subjects diagnosed or suspected of having a lung sarcoma are administered one or more therapeutically effective doses of an antibody disclosed herein. The lung sarcoma is ameliorated or eliminated following treatment.

### Example 5—Reduction of Fibrotic Pathology in Subjects with a Primary Fibrotic Disease

Subjects diagnosed or suspected of having a primary fibrotic disease (e.g., interstitial lung disease (ILD), idiopathic pulmonary fibrosis (IPF), diffuse interstitial lung disease, liver fibrosis, and cirrhosis) are administered one or more therapeutically effective doses of an antibody disclosed herein. Fibrotic pathology is ameliorated or eliminated following treatment.

### Example 6—Reduction of Fibrotic Pathology in Subjects with Fibrosis as a Secondary Disease

Subjects diagnosed or suspected of having fibrosis as a complication of a non-fibrotic disease (e.g., an infection, an autoimmune disease or disorder, cancer, or an inflammatory disease or disorder) and determined or suspected to have M2 macrophages present in fibrotic tissue are administered one or more therapeutically effective doses of an antibody disclosed herein. Fibrotic pathology is ameliorated or eliminated following treatment.

### Example 7—Reductone of Fibrotic Pathology in Subjects with Fibrosis Resulting from Tissue Injury

Subjects diagnosed or suspected of having fibrosis as a complication of a tissue injury (e.g., a radiation induced injury or a mechanical injury) and determined or suspected to have M2 macrophages present in the injured tissue are administered one or more therapeutically effective doses of an antibody disclosed herein. Fibrotic pathology is ameliorated or eliminated following treatment.

### Example 8—a High Affinity Variant of AB101 Demonstrates Stronger Binding to CD163 on SU-DHL-1 WT Cells

DHL-1 (ATCC; No. CRL-2955) is a phagocytic human anaplastic large cell lymphoma cell line that constitutively express CD163 but lack expression of Fc receptors (FcRs) on the cell surface. These cells were cultured in suspension in RPMI-1640 media (HyClone; No. SH30027.02) supplemented with 10% fetal bovine serum (HyClone; No. SH30396.03).

To evaluate the role of FcRs in the binding of AB101 and its high affinity variants. CD163-expressing SU-DHL-1 cells were transduced with lentiviruses encoding human CD64 or FcγRI (Origene; No. RC207487L3V), CD32a or FcγRIIa (G&P Biosciences; No. LTV2124P), and CD16 or FcγRIII (Origene; No. RC206429L3V). SU-DHL-1 (5×10⁴) cells were treated with lentiviral particles encoding respective FcRs (CD16, CD32a, and CD64) at 20-50 particles per cell in the presence of 8 μg/mL polybrene (Millipore; No. TR-1003-G) to generate SU-DHL-1 cell lines with stable FcR expression. Cells were infected at room temperature (RT) by centrifugation for 1 hour at 800×g and subsequently transferred to an incubator (37° C., 5% CO2) for 4 hours. Virus-containing media was then removed, and cells were allowed to recover in fresh media for three days prior to selection in media containing 0.3 μg/mL of puromycin (ThermoFisher; No. A1113803). The lentiviral particles delivered the respective FcR genes into the target cells upon infection and the contents were then integrated at high efficiency into the host genome. By selecting for stable FcR expression utilizing a puromycin as a selection marker, populations of CD16, CD32, and CD64 expressing cells were isolated and cultured. Expression of FcRs on puromycin-resistant cell population was confirmed by flow cytometry using BD FACSymphony cytometer and FcR-specific antibodies.

Binding of AB101, a representative variant, V3, and hIgG1 isotype control to SU-DHL-1 wild-type (WT) or FcγR expressing cells was evaluated by flow cytometry. The isotype control was a proprietary human IgG1 mAb, with known specificity and identical IgG1 Fc region to AB101 and V3. SU-DHL-1 cells were washed once with PBS and resuspended in Zombie UV live/dead stain (BioLegend, No. 423107)(1:500) at RT for 20 minutes. Cells were then washed with FACS buffer (PBS+1% FBS+1 mM EDTA (Fisher Scientific, No. 15575-038)) and resuspended in FACS Block (FACS buffer containing 10% FBS and 0.5 mg/mL hIgG1) at RT for 20 minutes. Cells in blocking buffer were transferred to a 384-well plate at 2.5×10⁴ cells/well and titrated antibodies (AB101, V3, and hIgG1 isotype control) were added directly to each well at 2× final assay concentration. Cells were incubated with antibodies at RT for 20 minutes. Cells were washed three times with FACS buffer, then resuspended in FACS buffer for acquisition on a FACSymphony™ Cytometer (BD Biosciences).

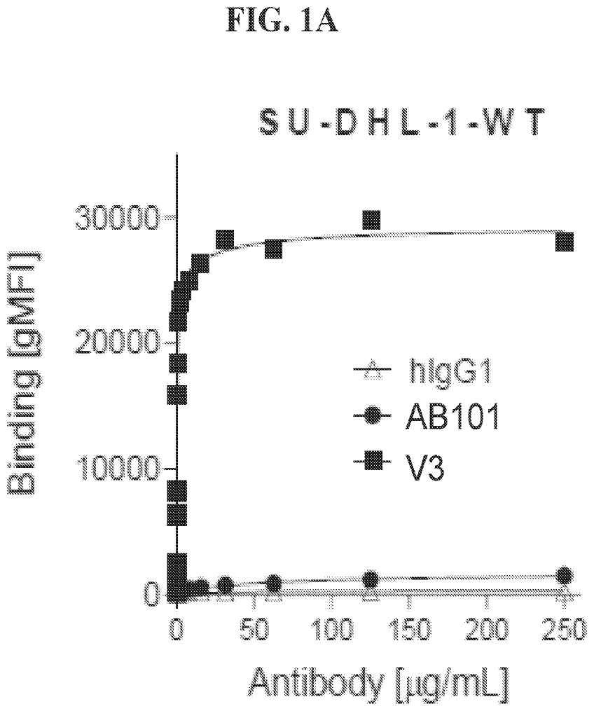

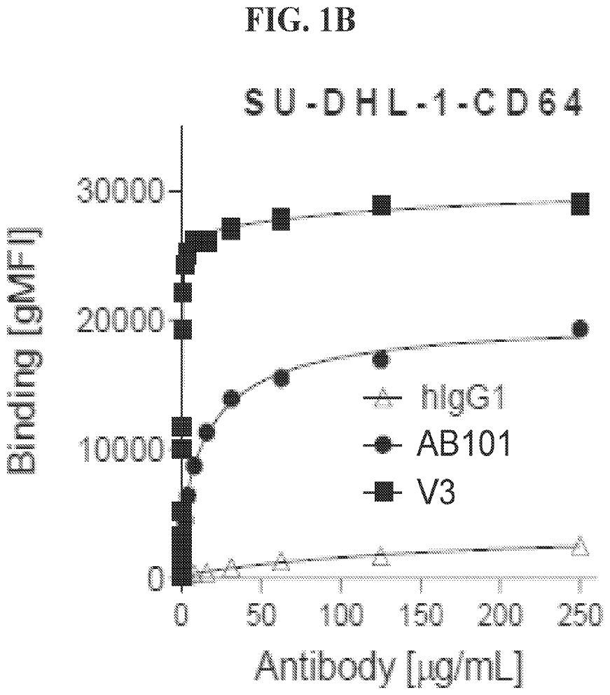

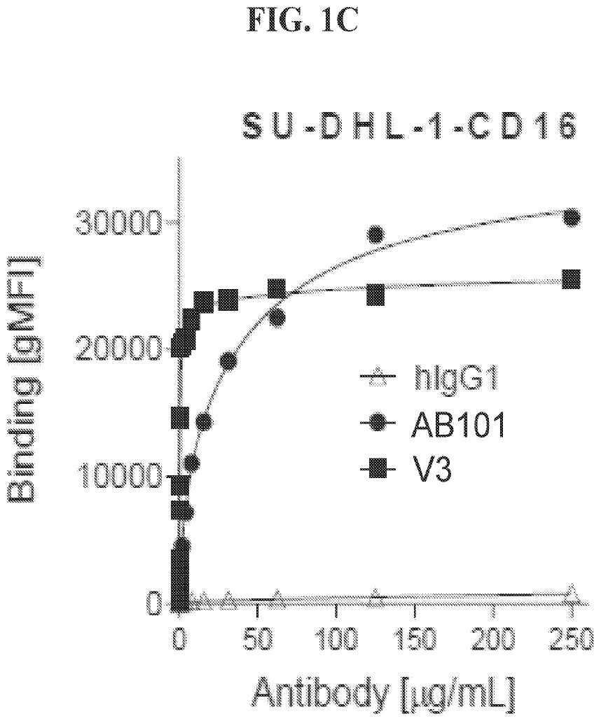

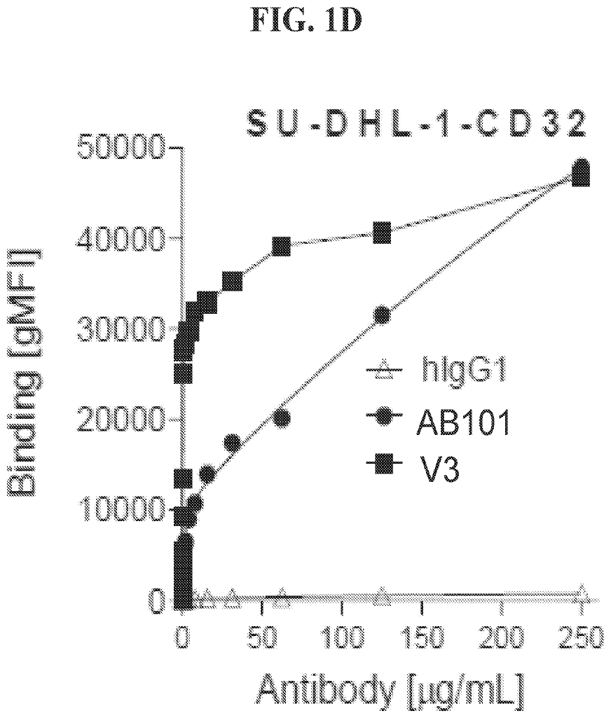

Although SU-DHL-1 wild-type cells express CD163 constitutively, no appreciable AB101 binding was observed (FIG. 1A). Optimal binding of AB101 to SU-DHL-1 cells in this assay requires co-expression of an Fc receptor, e.g., FcγRIII (FIG. 1B), FcγRI (FIG. 1C), or FcγRII (FIG. 1D). By contrast, V3 binds well to CD163 expressed on the wild type cells even in the absence of expressed Fc receptor (FIG. 1A), and this binding is not significantly altered by co-expression of FcγRs (FIGS. 1B-1D). The hIgG1 control antibody showed negligible or no binding in these assays, suggesting that Fc receptor binding alone is not sufficient to demonstrate measurable binding of IgG1 antibodies in this assay.

### Example 9—High Affinity Variants of AB101 Demonstrate Stronger Binding to Recombinant CD163 Protein by ELISA

CD163 variant antibody binding to His-tagged recombinant human CD163 (R&D Systems, No. 1607-CD-050) protein was determined using ELISA. Recombinant protein was diluted in PBS to 5 μg/mL and added to 384-well high-binding ELISA plates (Greiner Bio-One, No. 781061) at 25 μL per well and incubated at 4° C. overnight. The plates were washed three times with PBS, using a BioTek ELx405 Select microplate washer (wash program ELISA_384_PBS_3×_wash) and then blocked with 90 μL/well of blocking buffer (2% nonfat, dry milk/PBS+0.05% Tween 20) for 1 hour at RT.

After blocking, 25 μL per well of primary antibodies were added to the plates and incubated for 1 hour at RT. The isotype control was a proprietary mAb, in human IgG1 frameworks, with known specificity. After primary antibody binding, plates were washed three times with PBS using the EL405× (wash program ELISA_384_PBS 3×_wash). The secondary antibody was a goat anti-human IgG F(ab′)₂ HRP (Jackson Immunoresearch, No. 109-035-097). Secondary antibody was diluted to 1:2500 in 2% nonfat, dry milk/PBS, and 25 μL per well was added to the wells and incubated at RT for 1 hour. The plates were washed four times with PBS, using the EL405× (wash program ELISA_384_PBS_4× wash). After removal of the final wash, 25 μL/well of neat I-Step™ Ultra TMB-ELISA Substrate Solution (ThermoFisher, No. 34028) was added and the plates were incubated for 10-15 minutes at RT protected from light. After development, the reaction was stopped by adding 25 μL per well of 0.3 M HCl and plates were read using a SpectraMax M5e instrument at 450 nm.

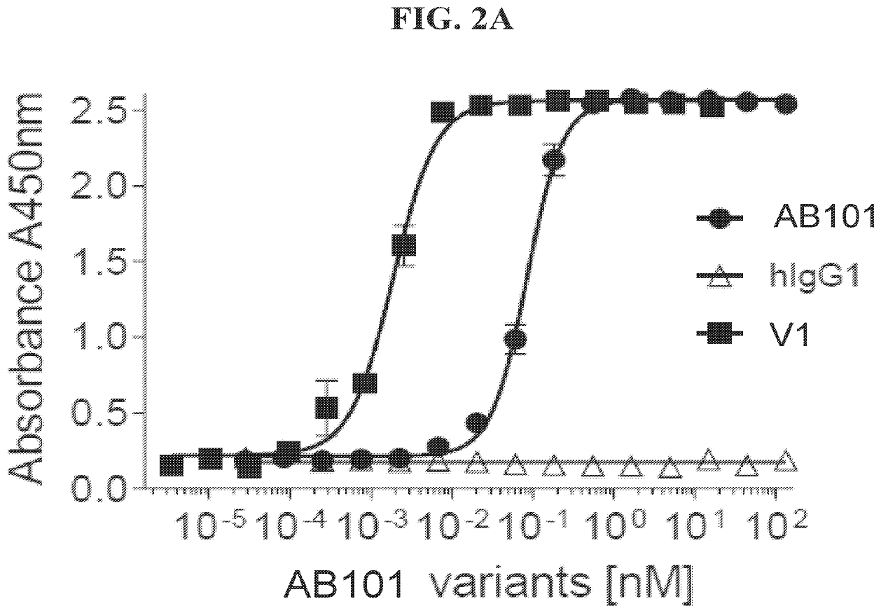

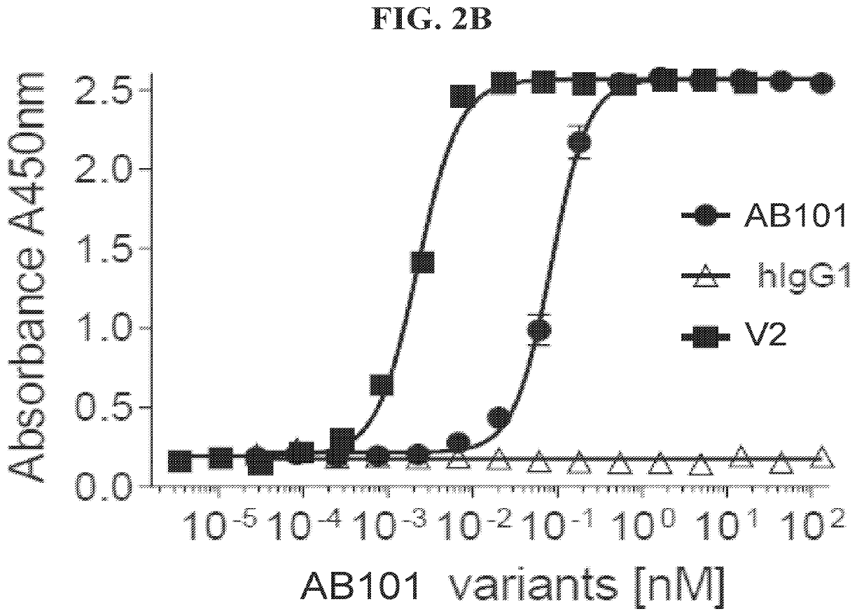

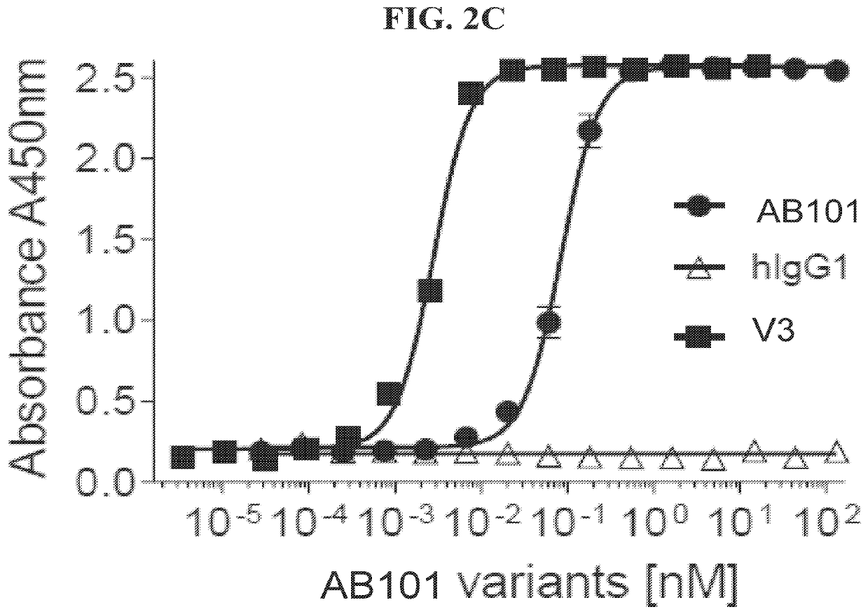

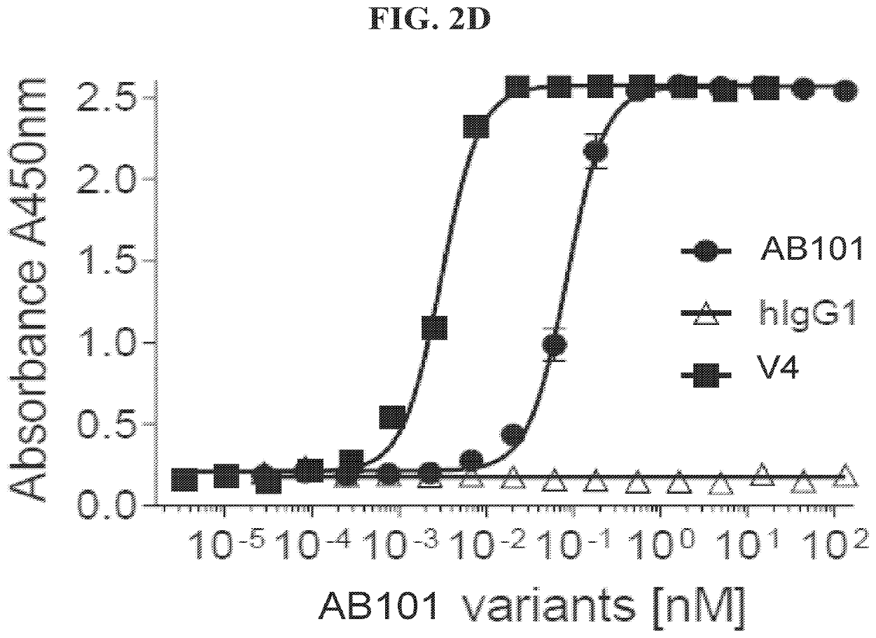

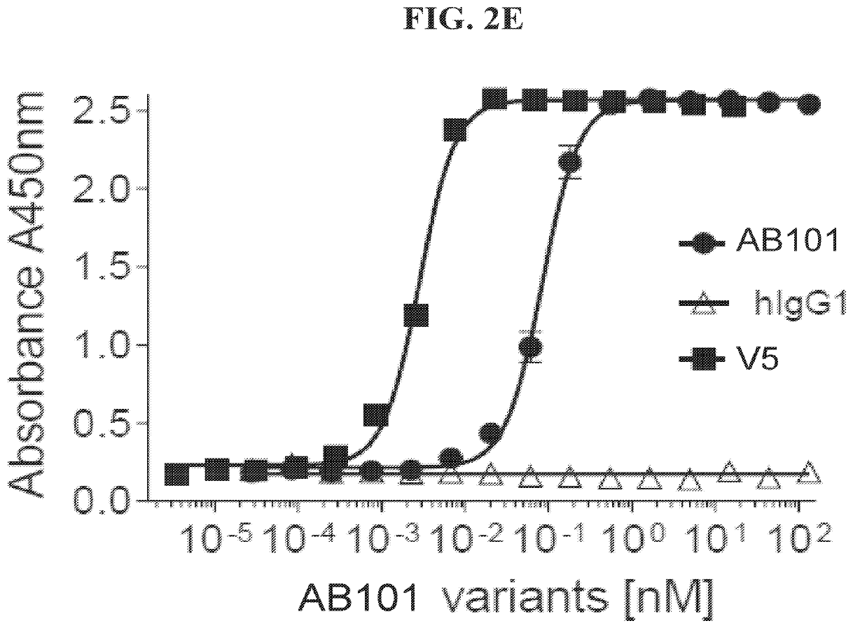

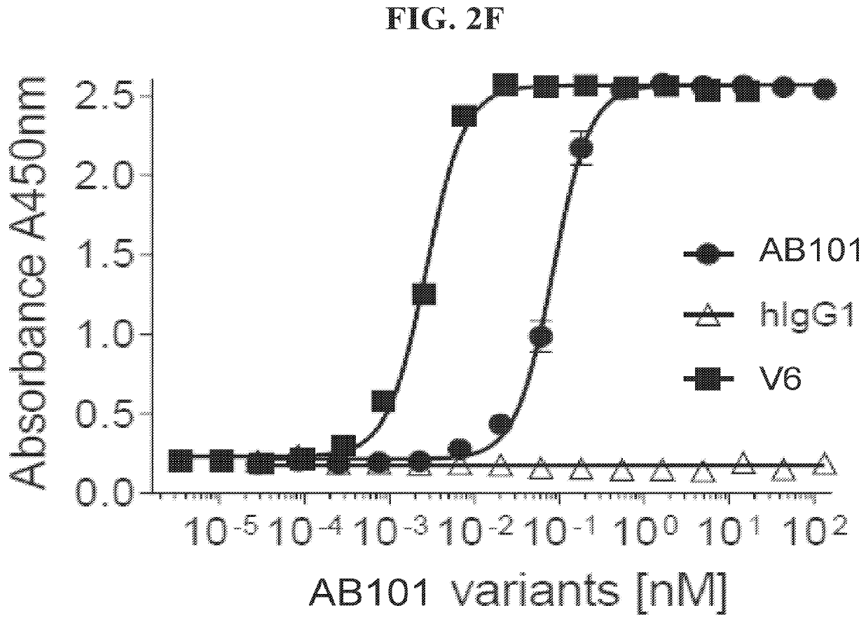

As shown in FIGS. 2A-2F, the variants of AB101, namely V1 (FIG. 2A), V2 (FIG. 2B), V3 (FIG. 2C), V4 (FIG. 2D), V5 (FIG. 2E), and V6 (FIG. 2F), and the AB101 parental antibody bound to huCD163 in this assay, while the isotype control showed no appreciable binding. The EC₅₀ value of AB101 parental antibody binding to huCD163 was 84.39 pM, whereas the EC₅₀ value of variants of AB101 ranged from 1.87 pM to 3.05 pM demonstrating 27- to 45-fold stronger binding than AB101 parental antibody (Table 7).

TABLE 7
EC50 values for antibody binding to huCD163

| Antibody | EC50 (nM) |
|---|---|
| AB101 | 84.39 |
| V1 | 1.87 |
| V2 | 2.23 |
| V3 | 2.79 |
| V4 | 3.05 |
| V5 | 2.83 |
| V6 | 2.68 |

### Example 10: Preparation of Isolated Monocytes from Donor Samples

Apheresis products may be collected from donors and autologous monocytes and T cells are isolated using techniques commonly used in the art. Here, human monocytes and T cells are isolated from white blood cells (WBCs) according to standard techniques. WBCs are trapped within an integrated leukoreduction system (LRS) chamber (Trima, No. 2490-08) during the collection process or LeukoPaks (No. 4510-01, Full LeukoPak, BloodWorks Northwest, Seattle, WA). Peripheral blood mononuclear cells (PBMCs) are purified from the LRS chambers or LeukoPaks by standard density gradient centrifugation (FicollPaque®, Premium 1.073, GE Healthcare, No. 17-5449-52). The supernatant is discarded, and the pellet resuspended in 20 mL EasySep™ Buffer (STEMCELL Technologies, No. 20144) for counting of PBMCs and for further isolation of monocytes and T cells.

Monocytes are isolated using the EasySep™ Human Monocyte Isolation kit (STEMCELL Technologies, No. 19359) following the manufacturer's instructions.

Total CD3+, CD4+, and CD8+ T cells can be isolated using the EasySep™ Human CD3+, CD4+, and CD8+ T Cell Isolation kits (STEMCELL Technologies, Nos. 19051, 17952, and 17953), following the manufacturer's instructions. These negative selection kits use antibodies to label undesired cell types for removal, allowing the desired target cells to be isolated from the sample untouched.

Macrophages can be generated from PBMC-derived monocytes using techniques commonly used in the art, such as those exemplified below.

Generation of M0 Macrophages: At day 0, monocytes from individual donors are plated in 96-well culture plates (ThermoFisher (Costar), No. 09-761-145) at 25,000-50,000 cells/100 μL/well in M0 culture medium (90% X-VIVO™ 15 (Lonza No. 04-418Q)+10% heat-inactivated FBS (Hyclone, No. SH30396.03)+100 ng/mL human M-CSF (PeproTech, No. 300-25)). Cells are incubated at 37° C. and 5% CO₂ for 5 to 6 days to produce M0 macrophages.

Generation of M2c Macrophages: At Day 5-6 of culture, the M0 macrophages are polarized to M2c macrophages by aspirating the medium gently from each plate and replacing it with 100 μL/well of M2c culture medium (M0 culture medium with 20 ng/mL human IL-10 (PeproTech, No. 200-10)). Cells are incubated at 37° C. and 5% CO₂ for 2 days. At Day 7-8 of culture, the M2c macrophages are ready for assay setup.

### Example 11—High Affinity Variants of AB101 Demonstrate Stronger Binding to M2c Macrophages by FACS

To assess binding kinetics of variant antibodies to CD163 expressed on cells, binding studies were performed with immunosuppressive M2c macrophages. Macrophages were isolated and prepared according to standard methods, such as, e.g., those described in Example 10, above.

M2c cells were incubated for 15 minutes at RT in Macrophage Detachment Solution DXF (PromoCell, No. C-41330) and removed from the flask into X-VIVO™ 15 serum-free medium (Lonza, No. 04-418Q).

Following centrifugation, the cells were washed once with PBS and resuspended in Zombie UV live/dead stain (BioLegend, No. 423107)(1:500) at RT for 20 minutes. Cells were then washed with FACS buffer (PBS+1% FBS+1 mM EDTA (Fisher Scientific No. 15575-038)) and resuspended in FACS Block (FACS buffer containing 10% FBS and 0.5 mg/mL human IgG1) at RT for 20 minutes. Cells in blocking buffer were transferred to a 384-well plate at 2.5×10⁴ cells/well and titrated antibodies were added directly to each well at 2× final assay concentration. Cells were incubated with antibodies at RT for 20 minutes. Cells were washed three times with FACS buffer, then resuspended in FACS buffer for acquisition on a FACSymphony™ Cytometer (BD Biosciences).

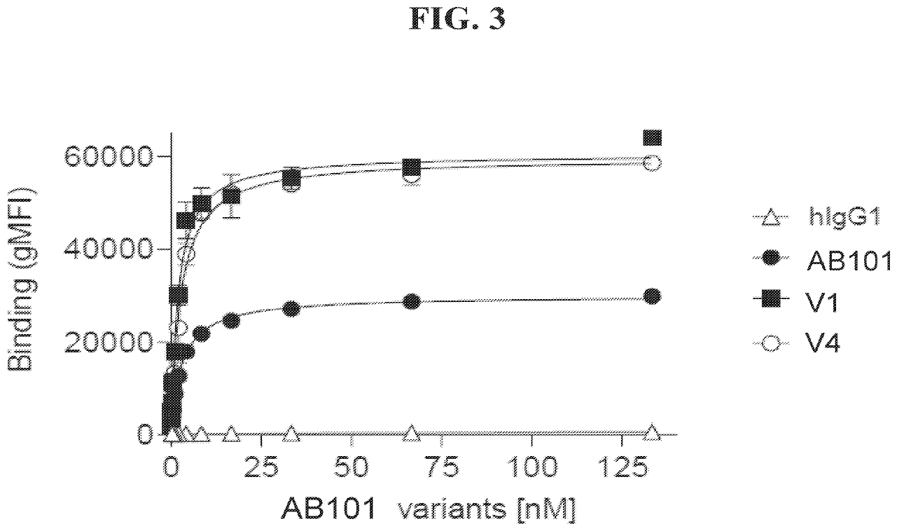

As shown in FIG. 3, these representative variants showed improved binding to M2c macrophages as compared to the parental AB101 antibody. The human IgG1 (hIgG1) isotype control exhibited no binding to M2c macrophages.

### Example 12—a High Affinity Variant of AB101 Retains Specificity and Shows Improved Binding to CD163+ Cells in Whole Blood

Antibody binding to human immune cell lineage subsets was assessed by flow cytometry. Briefly, whole blood with heparin as an anticoagulant was obtained (Bloodworks Northwest). Fc-receptor blocking reagent was directly added to whole blood to a final concentration of 10% FBS, 0.5 mg/mL human IgG1 and 0.05% NaN₃ in a final sample volume of 400 μL. Following 20 minutes of blocking at RT, APC-labeled anti-CD163 antibodies AB101, V3, human IgG1 isotype control, anti-CD163 clone R20, and murine IgG1 isotype control were added. After 30 minutes, red blood cells (RBC) were lysed with RBC lysis buffer, followed by Zombie UV viability staining. Then, cells were washed with FACS buffer (PBS containing 1% FBS, 2 mM EDTA, and 0.05% NaN₃) and resuspended in FACS buffer containing 10% FBS and 10% FcX block. Then, the antibody phenotyping cocktail (Table 8a) was added and incubated at room temperature for 30 minutes in the dark. Cells were washed with FACS buffer and resuspended in FACS buffer data acquisition using a FACSymphony™ Cytometer (BD BioSciences). Analysis was performed using FlowJo software. Populations were identified according to gating parameters as described in Table 8b.

TABLE 8a
Antibody phenotyping cocktail

| Marker | Antibody Clone | Fluorophore |
|---|---|---|
| CD3 | UCHT-1 | BV421 |
| CD8 | SK1 | APC-Cy7 |
| CD4 | RPA-T4 | BV711 |
| HLA-DR | L243 | PE-Cy7 |
| CD19 | H1B9 | BV605 |
| CD56 | HCD56 | FITC |
| CD14 | MφP9 | BB700 |
| CD16 | 3G8 | BV786 |
| CD11c | Bu15 | PE |
| CD15 | W6D3 | BUV805 |

TABLE 8b
Immune cell lineages

| Cell Type | Gates |
|---|---|
| CD4⁺ T cells | CD3⁺CD4⁺ |
| CD8⁺ T cells | CD3⁺CD8⁺ |
| B cells | CD19⁺HLA-DR⁺ |
| NK cells | CD3⁻CD56⁺ |
| Neutrophils | SSC^{high}CD15⁺ |
| Classical monocytes | CD14⁺HLA-DR⁺CD16⁻ |
| Intermediate monocytes | CD14⁺HLA-DR⁺CD16⁺ |
| Non-classical monocytes | CD14⁻HLA-DR⁺CD16⁺ |
| Myeloid dendritic cells | CD14⁻HLA-DR⁺CD11c⁺ |

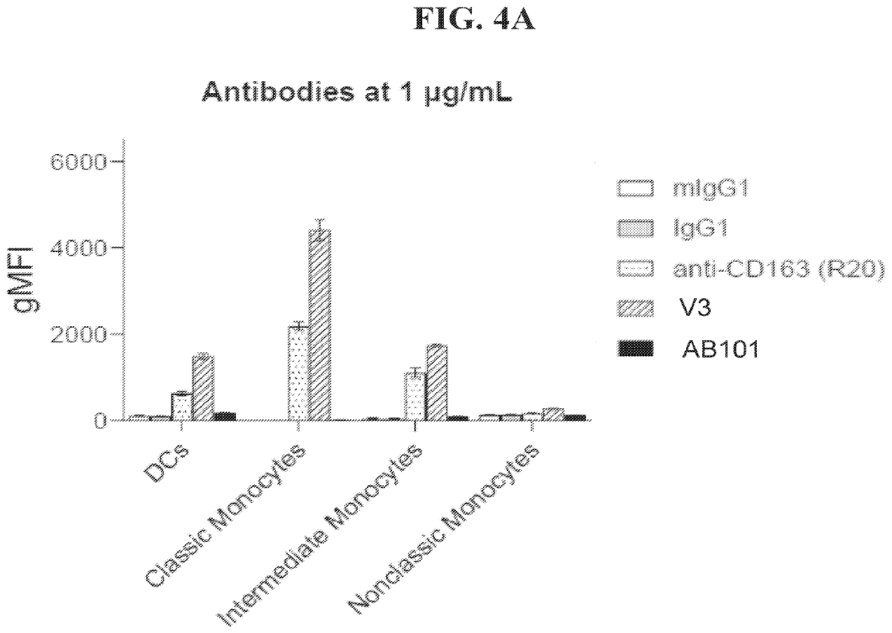

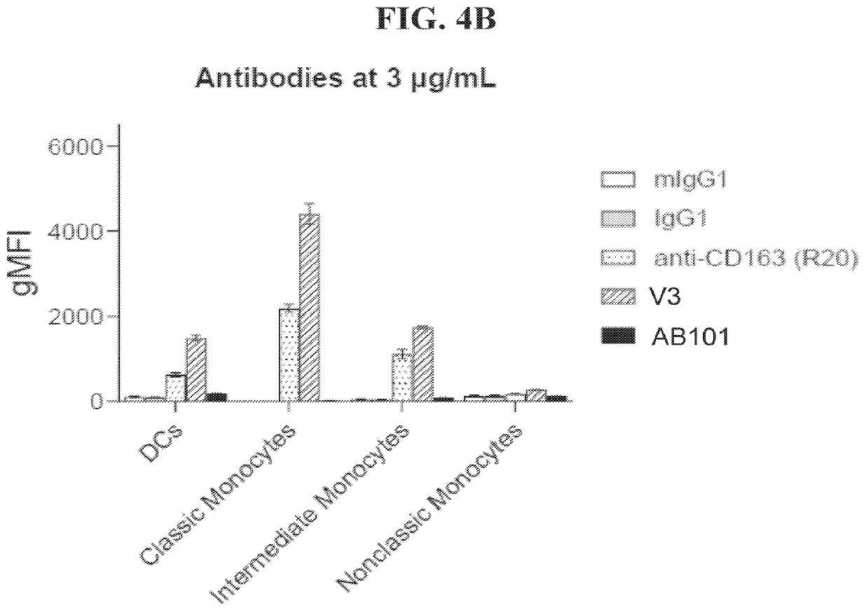

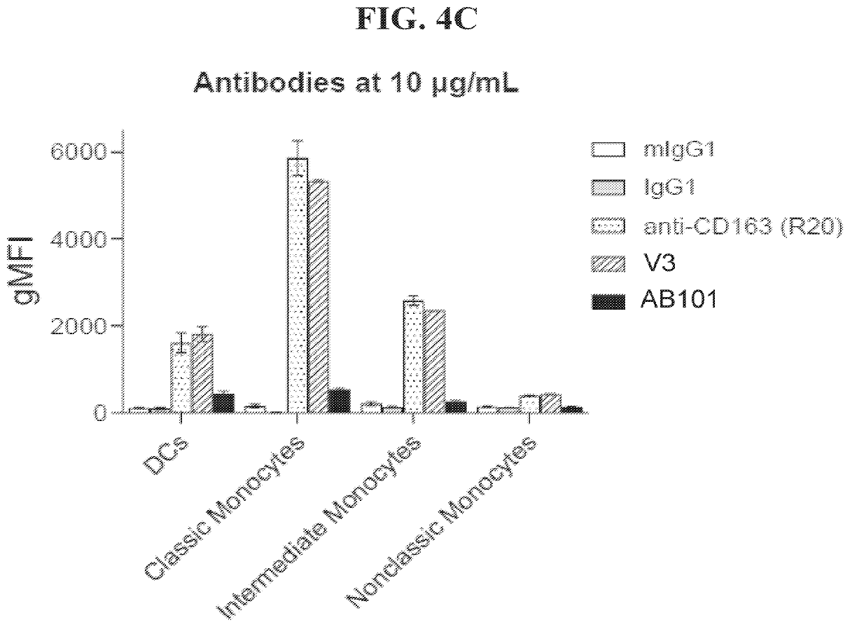

As shown in FIGS. 4A-4C, CD163 expression was detected on classical monocytes, intermediate monocytes, and a portion of myeloid dendritic cells (mDCs) using commercially available anti-CD163 clone R20 (R&D Systems, No. FAB16072R10). A comparison of staining using the anti-CD163 clone R20 across monocytes and DCs showed that CD163 expression varied across cell types (classical monocytes>intermediate monocytes>mDCs). No appreciable binding to CD4+ and CD8+ T cells, B cells, NK cells, neutrophils, non-classical monocytes, and neutrophils was observed (data not shown).

Consistent with CD163 expression, AB101 and V3 bound specifically to monocytes, without appreciable binding to human neutrophils, B cells, NK cells, CD4+ T cells, or CD8+ T cells. The human IgG1 isotype control (IgG1) did not show detectable binding to any of the evaluated immune cell lineages. V3 demonstrated improved binding to CD163+ cells in whole blood. The geometric MFI of V3 binding to DCs, classical monocytes, intermediate monocytes, and nonclassical monocytes was increased by 4.3-, 9.8-, 9.2-, and 3-fold, compared to gMFI for AB101 (FIGS. 4A-4C).

### Example 13A—CD163 Antibodies Relieve M2c-Mediated Immune Suppression in M2c/CD8+ T Cell Coculture as Measured by T Cell Proliferation

Suppression of T cells in the tumor microenvironment by tumor-associated macrophages contributes to the immunosuppressive tumor microenvironment. This activity can be modeled by co-culture of autologous monocyte-derived M2 macrophages with activated CD8+ or CD4+ T cells, and T cell proliferation and cytokine expression levels can be used as surrogates for T cell activation.

After polarization of M0 macrophages to M2c macrophages, supernatants were removed from M2c macrophage 96-well culture plates and replaced with 100 μL of assay medium (X-VIVO™ 15 media+10% FBS) containing 0.625 μg/mL OKT3 (ThermoFisher, No. 14-0037-82)+/−CD163 antibodies and incubated at 37° C., 5% CO2 for 1-2 hours. While the M2c macrophages were contacted with the antibodies, autologous CD8+ T cells isolated as described above were labeled with 5 μM CellTrace™ Violet Proliferation Dye (ThermoFisher, No. C34571) for 20 minutes at 37° C., 5% CO₂. Excess CellTrace™ was washed off with ice-cold assay medium, labeled CD8+ T cells were resuspended in assay medium, and then added to the M2c/antibodies preparation at 1:1 ratio of M2c:CD8+ T cells. Cells were then incubated at 37° C., 5% CO₂ for 72 hours.

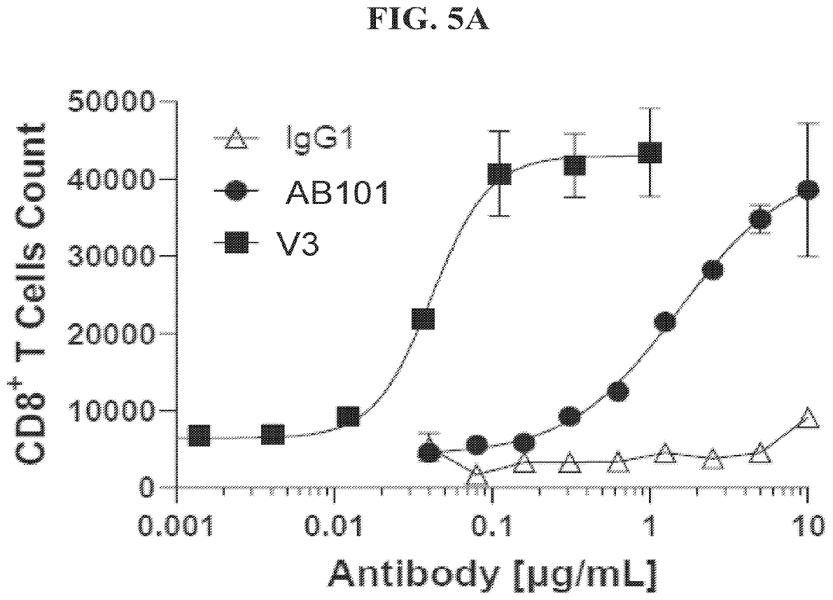

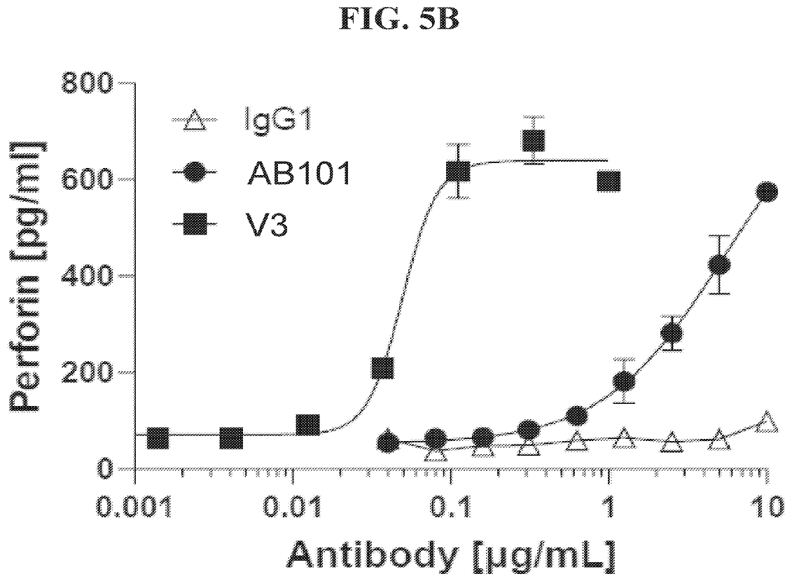

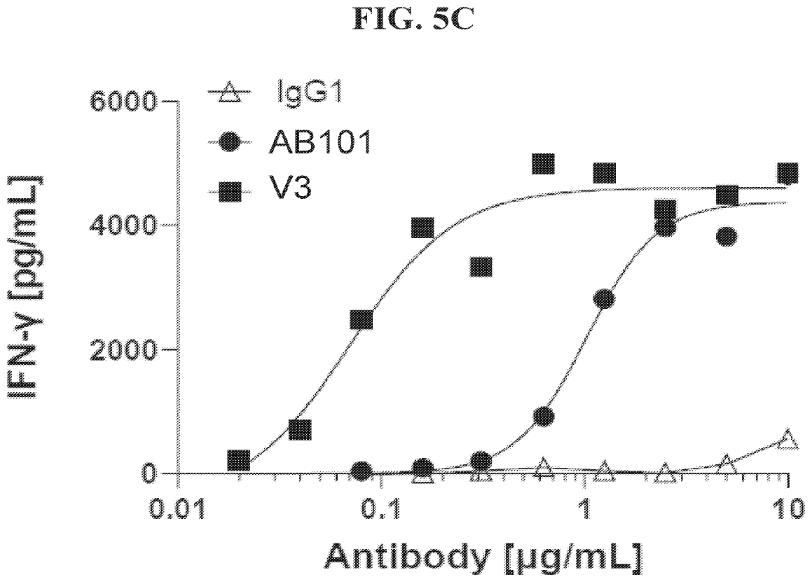

Supernatants containing T cells were transferred to a V-bottom 96-well plate, centrifuged to pellet the T cells, and the culture supernatants were collected and frozen at −20° C. for human IFNγ cytokine analysis by ELISA (R&D Systems; Duoset ELISA, No. DY285B). T cell pellets were blocked with True Stain Fc block (BioLegend) and stained with anti-human CD8-BUV395 (BD Biosciences) for 20 minutes at 4° C. Cells were washed and stained with e780 viability dye for 15 minutes at RT in the dark, washed with FACS buffer, and resuspended in 200 μL of FACS buffer acquisition on BD Symphony flow cytometer (BD Biosciences). The percentage of proliferating CD8+ T cells was analyzed using FlowJO software and reported as percent CD8+ CellTrace-negative dividing cells. The CD163 antibodies relieved M2c-macrophage-mediated immune suppression in M2c/T cell coculture assays by restoring CD8+ T-cell proliferation (FIG. 5A). V3 was approximately 40-fold more potent than the parental AB101 antibody in relieving the M2c-mediated suppression of CD8+ T cell proliferation (Table 9).

TABLE 9
EC50 values antibody-elicited T cell proliferation

| Antibody | EC50 (nM) |
|---|---|
| AB101 | 10.10 |
| V3 | 0.27 |

### Example 13B—CD163 Antibodies Relieve M2c-Mediated Immune Suppression In M2c/CD8+ T Cell Coculture as Measured by Perforin Secretion

Antibody treatment during M2c/T cell coculture relieved M2c mediated immunosuppression and induced a potent cytotoxic response by anti-CD3 activated CD8+ T cells. CD8+ T cells isolated from 2 to 3 study subjects were activated with anti-CD3 (OKT3, 0.25 μg/mL) in the presence of M2c macrophages. M2c/T cell cocultures were treated with 20 μg/mL of anti-CD163 antibody, human IgG1 isotype control, or media alone (M2c). Supernatants were taken 72 hours after anti-CD3 stimulation and perforin secretion was quantified by magnetic bead-based immunoassay. In this assay the CD163 antibodies rescued the perforin response of exhausted T cells from M2c macrophage-mediated immune suppression (FIG. 53), while the control IgG1 antibody showed no activity. The V3 antibody was approximately 100-fold more potent than the parental AB101 antibody in relieving the M2c-mediated suppression of perforin secretion by the CD8+ T cells (Table 10).

TABLE 10
EC50 values antibody-elicited perforin secretion

| Antibody | EC50 (nM) |
|---|---|
| AB101 | 36.45 |
| V3 | 0.33 |

### Example 13C—CD163 Antibodies Relieve M2c-Mediated Immune Suppression in M2c/T Cell Blast Coculture as Measured by IFNγ Secretion

Most T cells in the tumor microenvironment are exhausted, showing decreased cytokine expression and effector function, which contributes to cancer immune evasion. Reversing T-cell exhaustion and restoring anti-tumor potential represents a promising strategy to treat cancer. We evaluated the ability of CD163 antibodies to rescue functional activity of exhausted T cells from immune suppression using a coculture of exhausted T cells and M2c, a system that models the immune suppression mediated by macrophages. Exhausted T cells having a blast-like morphology can be generated from human PBMCs by repeated (3×) phytohemagglutinin (PHA) stimulation.

Frozen PBMCs or freshly isolated PBMCs were incubated at 1×10⁶ cells/mL in culture medium (IMDM (Thermo Fisher, No. 12440053)+10% human AB serum (Sigma Aldrich, No. H4522)+2 μg/mL PHA-L (Sigma Aldrich, No. 11249738001)+4 ng/mL recombinant human IL-2 (R&D Systems, No. 202-IL)) at 37° C., 5% CO₂ for 10 days, with cell passage and media change on days 4 and 7. On Day 10, T cell blasts were collected using standard methods.

To measure the ability of the CD163 antibodies to rescue functional activity of T cell blasts from M2c-mediated immune suppression, old medium from cultured M2c macrophages was removed and replaced with 50 μL of X-VIVO™ 15 medium+10% FBS containing 4× final concentration of CD163 or isotype control antibodies and incubated at 37° C., 5% CO₂ for 2 hours. OKT3 antibody was diluted to 0.25 pg/mL and added to the M2c/antibody samples, and incubated at 37° C., 5% CO₂ for 30 minutes. T cell blasts were added last to the samples at a 1:1 ratio of T cell blasts to M2c and incubated at 37° C., 5% CO₂ for 24 hours. Supernatants were collected for human interferon γ (IFNγ) cytokine analysis by ELISA. Each treatment condition was set up in triplicate.

In this assay, the CD163 antibodies rescued the IFNγ response of exhausted T cells from M2c macrophage-mediated immune suppression (FIG. 5C), while the control IgG1 antibody showed no activity. V3 appeared to be over 10-fold more potent than the AB101 antibody in relieving the M2c-mediated suppression of IFNγ release by the CD8 T cells (Table 11).

TABLE 11
EC50 values antibody-elicited IFNγ secretion

| Antibody | EC50 (nM) |
|---|---|
| AB101 | 6.84 |
| V3 | 0.47 |

What is claimed is:

1. An antibody, comprising:
(a) a heavy chain variable region (V_{H}) having a sequence at least 80% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, AND SEQ ID NO: 41; and
(b) a light chain variable region (V_{L}) having a sequence at least 80% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40;
provided that the antibody does not comprise a light chain variable region (V_{L}) having a sequence as set forth in SEQ ID NO: 40 and a heavy chain variable region (V_{H}) having a sequence as set forth in SEQ ID NO. 41.

2. The antibody of claim 1, wherein the light chain variable region (V_{L}) has a sequence at least 85% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

3. The antibody of claim 1, wherein the light chain variable region (V_{L}) has a sequence at least 90% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

4. The antibody of claim 1, wherein the light chain variable region (V_{L}) has a sequence at least 95% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

5. The antibody of claim 1, wherein the light chain variable region (V_{L}) has a sequence at least 99% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

6. The antibody of claim 1, wherein the light chain variable region (V_{L}) has a sequence 100% identical to the amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

7. The antibody of claim 1, wherein the heavy chain variable region (V_{H}) has a sequence at least 85% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, AND SEQ ID NO: 41.

8. The antibody of claim 1, wherein the heavy chain variable region (V_{H}) has a sequence at least 90% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, AND SEQ ID NO: 41.

9. The antibody of claim 1, wherein the heavy chain variable region (V_{H}) has a sequence at least 95% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, AND SEQ ID NO: 41.

10. The antibody of claim 1, wherein the heavy chain variable region (V_{H}) has a sequence at least 99% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, AND SEQ ID NO: 41.

11. The antibody of claim 1, wherein the heavy chain variable region (V_{H}) has a sequence 100% identical to the amino acid sequence selected from the group consisting of: SEQ ID NO: 29, SEQ ID NO: 31, SEQ ID NO: 33, SEQ ID NO: 35, SEQ ID NO: 37, SEQ ID NO: 39, AND SEQ ID NO: 41.

12. The antibody of any one of claims 1-11, wherein the antibody is 100% identical at CDR H1, CDR H2, CDR H2, CDR L1, CDR L2, and CDR L3.

13. The antibody of any one of claims 1-12, wherein CDR H1 has a sequence as set forth in an amino acid sequence selected from the group consisting of: SEQ ID NO: 4, SEQ ID NO: 16, SEQ ID NO: 19, SEQ ID NO: 22 and SEQ ID NO: 25.

14. The antibody of any one of claims 1-13, wherein CDR H2 has a sequence as set forth in an amino acid sequence selected from the group consisting of: SEQ ID NO: 5, SEQ ID NO: 17, SEQ ID NO: 20, SEQ ID NO: 23, and SEQ ID NO: 26.

15. The antibody of any one of claims 1-14, wherein CDR H3 has a sequence as set forth in an amino acid sequence selected from the group consisting of: SEQ ID NO: 6, SEQ ID NO: 18, SEQ ID NO: 21, SEQ ID NO: 24, and SEQ ID NO: 27.

16. The antibody of any one of claims 1-15, wherein CDR L1 has a sequence as set forth in an amino acid sequence selected from the group consisting of: SEQ ID NO: 1, SEQ ID NO: 7, and SEQ ID NO: 13.

17. The antibody of any one of claims 1-16, wherein CDR L2 has a sequence as set forth in an amino acid sequence selected from the group consisting of: SEQ ID NO: 2, SEQ ID NO: 9, and SEQ ID NO: 14.

18. The antibody of any one of claims 1-17, wherein CDR L3 has a sequence as set forth in an amino acid sequence selected from the group consisting of: SEQ ID NO: 3, SEQ ID NO: 8, SEQ ID NO: 10, SEQ ID NO: 11, SEQ ID NO: 12, and SEQ ID NO: 15.

19. An antibody, comprising:
(a) a light chain CDR1 having an amino acid sequence at least about 80% identical to an amino acid sequence set forth in the group consisting of: SEQ ID NO: 1, SEQ ID NO: 7, and SEQ ID NO: 13; a light chain CDR2 having an amino acid sequence at least about 80% identical to an amino acid sequence set forth in the group consisting of: SEQ ID NO: 2, SEQ ID NO: 9, and SEQ ID NO: 14; and a light chain CDR3 having an amino acid sequence at least about 80% identical to an amino acid sequence set forth in the group consisting of: SEQ ID NO: 3, SEQ ID NO: 8, SEQ ID NO: 10, SEQ ID NO: 11, SEQ ID NO: 12, and SEQ ID NO: 15; and
(b) a heavy chain CDR1 having an amino acid sequence at least about 80% identical to an amino acid sequence set forth in the group consisting of: SEQ ID NO: 4, SEQ ID NO: 16, SEQ ID NO: 19, SEQ ID NO: 22 and SEQ ID NO: 25; a heavy chain CDR2 having an amino acid sequence at least about 80% identical to an amino acid sequence set forth in the group consisting of: SEQ ID NO: 5, SEQ ID NO: 17, SEQ ID NO: 20, SEQ ID NO: 23, and SEQ ID NO: 26; a heavy chain CDR3 having an amino acid sequence at least about 80%, identical to an amino acid sequence set forth in the group consisting of: SEQ ID NO: 6, SEQ ID NO: 18, SEQ ID NO: 21, SEQ ID NO: 24, and SEQ ID NO: 27;
provided that the antibody does not comprise a sequence at least as set forth in SEQ ID NO: 1, SEQ ID NO: 2, SEQ ID NO: 3, SEQ ID NO: 4, SEQ ID NO: 5, and SEQ ID NO: 6.

20. The antibody of claim 19, wherein the CDR L1 has a sequence at least 85% identical to the amino acid sequence selected from the group consisting of: SEQ ID NO: 1, SEQ ID NO: 7, AND SEQ ID NO: 13; the CDR L2 has a sequence at least 85% identical to the amino acid sequence selected from the group consisting of: SEQ ID NO: 2, SEQ ID NO: 9, AND SEQ ID NO: 14; and the CDR L3 has a sequence at least 85% identical to the amino acid sequence selected from the group consisting of: SEQ ID NO: 3, SEQ ID NO: 8, SEQ ID NO: 10, SEQ ID NO: 11, SEQ ID NO: 12, AND SEQ ID NO: 15.

21. The antibody of claim 19, wherein the CDR L1 has a sequence at least 90% identical to the amino acid sequence selected from the group consisting of: SEQ ID NO: 1, SEQ ID NO: 7, AND SEQ ID NO: 13; the CDR L2 has a sequence at least 90% identical to the amino acid sequence selected from the group consisting of: SEQ ID NO: 2, SEQ ID NO: 9, AND SEQ ID NO: 14; and the CDR L3 has a sequence at least 90% identical to the amino acid sequence selected from the group consisting of: SEQ ID NO: 3, SEQ ID NO: 8, SEQ ID NO: 10, SEQ ID NO: 11, SEQ ID NO: 12, AND SEQ ID NO: 15.

22. The antibody of claim 19, wherein the CDR L1 has a sequence at least 95% identical to the amino acid sequence selected from the group consisting of: SEQ ID NO: 1, SEQ ID NO: 7, AND SEQ ID NO: 13; the CDR L2 has a sequence at least 95% identical to the amino acid sequence selected from the group consisting of: SEQ ID NO: 2, SEQ ID NO: 9, AND SEQ ID NO: 14; and the CDR L3 has a sequence at least 95% identical to the amino acid sequence selected from the group consisting of: SEQ ID NO: 3, SEQ ID NO: 8, SEQ ID NO: 10, SEQ ID NO: 11, SEQ ID NO: 12, AND SEQ ID NO: 15.

23. The antibody of claim 19, wherein the CDR L1 has a sequence at least 99% identical to the amino acid sequence selected from the group consisting of: SEQ ID NO: 1, SEQ ID NO: 7, AND SEQ ID NO: 13; the CDR L2 has a sequence at least 99% identical to the amino acid sequence selected from the group consisting of: SEQ ID NO: 2, SEQ ID NO: 9, AND SEQ ID NO: 14, and the CDR L3 has a sequence at least 99% identical to the amino acid sequence selected from the group consisting of: SEQ ID NO: 3, SEQ ID NO: 8, SEQ ID NO: 10, SEQ ID NO: 11, SEQ ID NO: 12, AND SEQ ID NO: 15.

24. The antibody of claim 19, wherein the CDR L1 has a sequence 100% identical to the amino acid sequence selected from the group consisting of: SEQ ID NO: 1, SEQ ID NO: 7, AND SEQ ID NO: 13: the CDR L2 has a sequence 100% identical to the amino acid sequence selected from the group consisting of: SEQ ID NO: 2, SEQ ID NO: 9, AND SEQ ID NO: 14; and the CDR L3 has a sequence at least 100% identical to the amino acid sequence selected from the group consisting of: SEQ ID NO: 3, SEQ ID NO: 8, SEQ ID NO: 10, SEQ ID NO: 11, SEQ ID NO: 12, AND SEQ ID NO: 15.

25. The antibody of claim 19, wherein the CDR H1 has a sequence at least 85% identical to the amino acid sequence selected from the group consisting of: SEQ ID NO: 4, SEQ ID NO: 16, SEQ ID NO: 19, SEQ ID NO: 22 and SEQ ID NO: 25; the CDR H2 has a sequence at least 85% identical to the amino acid sequence selected from the group consisting of: SEQ ID NO: 5, SEQ ID NO: 17, SEQ ID NO: 20, SEQ ID NO: 23, and SEQ ID NO: 26, and the CDR H3 has a sequence at least 85% identical to the amino acid selected from the group consisting of: SEQ ID NO: 6, SEQ ID NO: 18, SEQ ID NO: 21, SEQ ID NO: 24, and SEQ ID NO: 27.

26. The antibody of claim 19, wherein the CDR H1 has a sequence at least 90% identical to the amino acid sequence selected from the group consisting of: SEQ ID NO: 4, SEQ ID NO: 16, SEQ ID NO: 19, SEQ ID NO: 22 and SEQ ID NO: 25; the CDR H2 has a sequence at least 90% identical to the amino acid sequence selected from the group consisting of: SEQ ID NO: 5, SEQ ID NO: 17, SEQ ID NO: 20, SEQ ID NO: 23, and SEQ ID NO: 26; and the CDR H3 has a sequence at least 90% identical to the amino acid selected from the group consisting of: SEQ ID NO: 6, SEQ ID NO: 18, SEQ ID NO: 21, SEQ ID NO: 24, and SEQ ID NO: 27.

27. The antibody of claim 19, wherein the CDR H1 has a sequence at least 95% identical to the amino acid sequence selected from the group consisting of: SEQ ID NO: 4, SEQ ID NO: 16, SEQ ID NO: 19, SEQ ID NO: 22 and SEQ ID NO: 25; the CDR H2 has a sequence at least 95% identical to the amino acid sequence selected from the group consisting of: SEQ ID NO: 5, SEQ ID NO: 17, SEQ ID NO: 20, SEQ ID NO: 23, and SEQ ID NO: 26; and the CDR H3 has a sequence at least 95% identical to the amino acid selected from the group consisting of: SEQ ID NO: 6, SEQ ID NO: 18, SEQ ID NO: 21, SEQ ID NO: 24, and SEQ ID NO: 27.

28. The antibody of claim 19, wherein the CDR H1 has a sequence at least 99% identical to the amino acid sequence selected from the group consisting of: SEQ ID NO: 4, SEQ ID NO: 16, SEQ ID NO: 19, SEQ ID NO: 22 and SEQ ID NO: 25; the CDR H2 has a sequence at least 99% identical to the amino acid sequence selected from the group consisting of: SEQ ID NO: 5, SEQ ID NO: 17, SEQ ID NO: 20, SEQ ID NO: 23, and SEQ ID NO: 26; and the CDR H3 has a sequence at least 99% identical to the amino acid selected from the group consisting of: SEQ ID NO: 6, SEQ ID NO: 18, SEQ ID NO: 21, SEQ ID NO: 24, and SEQ ID NO: 27.

29. The antibody of claim 19, wherein the CDR H1 has a sequence at least 100% identical to the amino acid sequence selected from the group consisting of: SEQ ID NO: 4, SEQ ID NO: 16, SEQ ID NO: 19, SEQ ID NO: 22 and SEQ ID NO: 25; the CDR H2 has a sequence at least 100% identical to the amino acid sequence selected from the group consisting of: SEQ ID NO: 5, SEQ ID NO: 17, SEQ ID NO: 20, SEQ ID NO: 23, and SEQ ID NO: 26; and the CDR H3 has a sequence at least 100% identical to the amino acid selected from the group consisting of: SEQ ID NO: 6, SEQ ID NO: 18, SEQ ID NO: 21, SEQ ID NO: 24, and SEQ ID NO: 27.

30. The antibody of claim 19, comprising:
(a) a heavy chain variable region (V_{H}) having a sequence at least 80% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO; 39, AND SEQ ID NO: 41; and
(b) a light chain variable region (V_{L}) having a sequence at least 80% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

31. The antibody of claim 19, comprising:
(a) a heavy chain variable region (V_{H}) having a sequence at least 85% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, AND SEQ ID NO: 41; and
(b) a light chain variable region (V_{L}) having a sequence at least 85% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

32. The antibody of claim 19, comprising:
(a) a heavy chain variable region (V_{H}) having a sequence at least 90% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, AND SEQ ID NO: 41; and
(b) a light chain variable region (V_{L}) having a sequence at least 90% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

33. The antibody of claim 19, comprising:
(a) a heavy chain variable region (V_{H}) having a sequence at least 95% identical to an amino acid sequence selected from the group consisting of SEQ ID NO: 29, SEQ ID NO: 31, SEQ ID NO: 33, SEQ ID NO: 35, SEQ ID NO: 37, SEQ ID NO: 39, AND SEQ ID NO: 41; and
(b) a light chain variable region (V_{L}) having a sequence at least 95% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

34. The antibody of claim 19, comprising:
(a) a heavy chain variable region (V_{H}) having a sequence at least 99% identical to an amino acid sequence selected from the group consisting of: SEQ ID NO: 29, SEQ ID NO: 31, SEQ ID NO: 33, SEQ ID NO: 35, SEQ ID NO: 37, SEQ ID NO: 39, AND SEQ ID NO: 41; and
(b) a light chain variable region (V_{L}) having a sequence at least 99% identical to an amino acid sequence selected from the group consisting of: SEQ ID NO: 28, SEQ ID NO: 30, SEQ ID NO: 32, SEQ ID NO: 34, SEQ ID NO: 36, SEQ ID NO: 38, AND SEQ ID NO: 40.

35. The antibody of claim 19, comprising:
(a) a heavy chain variable region (V_{H}) having a sequence 100% identical to an amino acid sequence selected from the group consisting of: SEQ ID NO: 29, SEQ ID NO:
31, SEQ ID NO: 33, SEQ ID NO: 35, SEQ ID NO: 37, SEQ ID NO: 39, AND SEQ ID NO: 41; and
(b) a light chain variable region (V_{L}) having a sequence 100% identical to an amino acid sequence selected from the group consisting of: SEQ ID NO: 28, SEQ ID NO:
30, SEQ ID NO: 32, SEQ ID NO: 34, SEQ ID NO: 36, SEQ ID NO: 38, AND SEQ ID NO: 40.

36. The antibody of any one of claims 1-35, wherein the antibody further comprises a human heavy chain constant region or a human light chain constant region.

37. The antibody of claim 36, wherein the human heavy chain constant region is IgG1 or IgG4 or a fragment thereof.

38. The antibody of any one of claims 1-37, wherein the antibody binds to an Fc receptor.

39. The antibody of claim 38, wherein the Fc receptor is expressed on the macrophage.

40. The antibody of any one of claims 1-39, wherein the antibody is an antibody fragment comprising a single heavy chain, a single light chain, Fab, F(ab′), F(ab′)₂, Fd, scFv, a variable heavy domain, a variable light domain, a variable NAR domain, bi-specific scFv, a bi-specific Fab₂, a tri-specific Fab₃ a single chain binding polypeptide, a dAb fragment, or a diabody.

41. The antibody of any one of claims 1-40, wherein the antibody binds to a dendritic cell.

42. The antibody of any one of claims 1-41, wherein the antibody binds to a classic monocyte.

43. The antibody of any one of claims 1-42, wherein the antibody binds to an intermediate monocyte.

44. The antibody of any one of claims 1-43, wherein the antibody binds to a nonclassic monocyte.

45. The antibody of any one of claims 1-44, wherein the antibody binds to an immunosuppressive myeloid cell.

46. The antibody of claim 45, wherein the immunosuppressive myeloid cell is in a tumor microenvironment.

47. The antibody of claim 45 or 46, wherein the immunosuppressive myeloid cell is a macrophage or a myeloid-derived suppressor cell.

48. The antibody of claim 47, wherein the human macrophage is an M2 macrophage or a M2-like macrophage.

49. The antibody of claim 47 or 48, wherein the human macrophage is an M2a, M2b, M2c, or M2d macrophage.

50. The antibody of any one of claims 47-49, wherein the macrophage is a tumor-associated macrophage.

51. The antibody of any one of claims 1-50, wherein the antibody binds to a CD163 protein.

52. The antibody of claim 51, wherein the CD163 protein is a glycoform of CD163.

53. The antibody of claim 52, wherein the CD163 protein is a 150 kDa glycoform of CD163.

54. The antibody of any one of claims 51-53, wherein the antibody does not specifically bind to a 130 kDa glycoform of CD163 expressed by the human macrophage.

55. The antibody of any one of claims 51-54, wherein the CD163 protein is a component of a cell surface complex comprising at least one other protein expressed by the macrophage.

56. The antibody of claim 55, wherein the at least one other protein is a galectin-1 protein, a LILRB2 protein, a casein kinase 11 protein, or any combination thereof.

57. The antibody of any one of claims 1-56, wherein the antibody specifically binds to a CD163 epitope comprising amino acid sequence SEQ ID NO: 42.

58. The antibody of any one of claims 1-56, wherein the antibody specifically binds to a CD163 epitope comprising amino acid sequence SEQ ID NO: 43.

59. The antibody of any one of claims 1-56, wherein the antibody specifically binds to a CD163 epitope comprising amino acid sequence SEQ ID NO: 44.

60. The antibody of any one of claims 1-56, wherein the antibody specifically binds to a CD163 epitope comprising each of amino acid sequence SEQ ID NO: 42, SEQ ID NO: 43, and SEQ ID NO: 44.

61. The antibody of any one of claims 51-60, wherein the antibody alters expression of at least one marker on the macrophage.

62. The antibody of claim 61, wherein at least one marker on the human macrophage is CD16, CD64, TLR2, or Siglec-15.

63. The antibody of any one of claims 1-62, wherein the antibody specifically binds to CD163 with a K_{D} from 0.5 nM to 100 nM.

64. The antibody of claim 63, wherein the antibody specifically binds to CD163 with a K_{D} from 0.5 nM to 50 nM.

65. The antibody of claim 64, wherein the antibody specifically binds to CD163 with a K_{D} from 0.5 nM to 10 nM.

66. The antibody of claim 65, wherein the antibody specifically binds to CD163 with a K_{D} from 0.5 nM to 1.5 nM.

67. The antibody of claim 66, wherein the antibody specifically binds to CD163 with a K_{D} from 0.5 nM to 1.0 nM.

68. The antibody of any one of claims 1-67, wherein the antibody specifically binds to human M2c macrophages with a K_{D} from 0.5 nM to 100 nM.

69. The antibody of claim 68, wherein the antibody specifically binds to human M2c macrophages with a K_{D} from 0.5 nM to 50 nM.

70. The antibody of claim 69, wherein the antibody specifically binds to human M2c macrophages with a K_{D} from 0.5 nM to 10 nM.

71. The antibody of claim 70, wherein the antibody specifically binds to human M2c macrophages with a K_{D} from 0.5 nM to 1.5 nM.

72. The antibody of claim 71, wherein the antibody specifically binds to human M2c macrophages with a K_{D} from 0.5 nM to 1.0 nM.

73. A method of treating a cancer or a fibrotic disease or disorder associated with a presence of M2-macrophages in a subject in need thereof, the method comprising administering to the subject a therapeutically effective amount of an antibody according to any one of claims 1-72.

74. The method of claim 73, wherein binding of the antibody to a macrophage promotes an immune cell function as measured by one or both of the following parameters:
(a) activation of a CD4+ T cell, CD8+ T cell, NK cell, or any combination thereof: and
(b) proliferation of a CD4+ T cell, CD8+ T cell, NK cell, or any combination thereof.

75. The method of claim 74, wherein the activation of a CD4+ T cell, CD8+ T cell, NK cell, or any combination thereof is measured as an enhanced level of IFN-γ, TNF-α, or perform, or any combination thereof.

76. The method of any one of claims 73-75, wherein binding of the antibody to a macrophage is not cytotoxic to the macrophage.

77. The method of any one of claims 73-76, wherein binding of the antibody to a macrophage results in at least one of the following effects:
(a) reduced expression of at least one marker by the macrophage, wherein the at least one marker is CD16, CD64, TLR2, or Siglec-15;
(b) internalization of the antibody by the macrophage;
(c) secretion of IFN-7, TNF-α, and perforin;
(d) activation of a CD4+ T cell, CD8+ T cell, NK cell, or any combination thereof:
(e) proliferation of a CD4+ T cell, CD8+ T cell, NK cell, or any combination thereof: and
(f) promotion of tumor cell killing in a tumor microenvironment.

78. The method of claim 77, wherein the binding results in: two or more of (a) through (e): three or more of (a) through (e): four or more of (a) through (e); or all of (a) through (e).

79. The method of any one of claims 73-78, wherein binding of the antibody to a macrophage increases an immunostimulatory activity in a tumor microenvironment.

80. The method of any one of claims 73-79, wherein binding of the antibody to a macrophage reduces an immunosuppression activity of the macrophage.

81. The method of any one of claims 73-80, wherein binding of the antibody to a macrophage reduces a tumor promoting activity of the macrophage.

82. The method of any one of claims 73-81, wherein binding of the antibody promotes CD4+ T cell activation, CD4+ T cell proliferation, or both CD4+ T cell activation and proliferation.

83. The method of any one of claims 73-82, wherein binding promotes expression of CD69, ICOS, OX40, PD1, LAG3, CTLA4, or any combination thereof by CD4+ T cells.

84. The method of any one of claims 73-83, wherein binding to the antibody promotes CD8+ T cell activation, CD8+ T cell proliferation, or both CD8+ T cell activation and proliferation.

85. The method of any one of claims 73-84, wherein binding of the antibody promotes expression of ICOS, OX40, PD1, LAG3, CTLA4, or any combination thereof by CD8+ T cells.

86. The method of any one of claims 73-85, wherein binding of the antibody protein promotes cytotoxic lymphocyte-mediated killing of cancer cells.

87. The method of any one of claims 73-86, wherein binding of the antibody promotes NK cell-mediated tumor cell killing.

88. The method of any one of claims 73-87, wherein binding of the antibody promotes expression of IL-2 by T cells.

89. The method of any one of claims 73-88, wherein binding of the antibody increases CD4+ T cells, CD196− T cells, CXCR3+ T cells, CCR4− T cells, or any combination thereof.

90. The method of any one of claims 73-89, wherein binding of the antibody to a macrophage reduces suppression of cytotoxic T cell-mediated killing of tumor cells in the tumor microenvironment.

91. The method of any one of claims 73-90, wherein the cancer is a lung cancer.

92. The method of any one of claims 73-91, wherein the cancer is a lung carcinoma or a lung sarcoma.

93. The method of any one of claims 73-92, wherein the cancer is a lung adenocarcinoma.

94. The method of any one of claims 73-93, further comprising administering to the subject an anti-cancer therapeutic.

95. The method of any one of claims 73-78, wherein binding of the antibody to a macrophage reduces the pro-fibrotic function of the macrophage.

96. The method of any one of claims 73-78, wherein the fibrotic disease or disorder is lung fibrosis.

97. The method of any one of claims 73-78, wherein the fibrotic disease or disorder is cardiac fibrosis.

98. The method of any one of claims 73-78, wherein the fibrotic disease or disorder is hepatic fibrosis.

99. The method of any one of claims 73-78, wherein the fibrotic disease or disorder is renal fibrosis.

100. The method of any one of claims 73-78, wherein the fibrotic disease or disorder is retinal fibrosis.

101. The method of any one of claims 73-78, wherein the fibrosis is a primary fibrotic disease or disorder.

102. The method of claim 101, wherein the primary fibrotic disease or disorder is idiopathic pulmonary fibrosis (IPF).

103. The method of claim 102, wherein the primary fibrotic disease or disorder is hepatic cirrhosis.

104. The method of claim 103, wherein the primary fibrotic disease or disorder is systemic sclerosis.

105. The method of claim 103, wherein the primary fibrotic disease or disorder is radiation fibrosis.

106. The method of claim 103, wherein the primary fibrotic disease or disorder is scarring associated with a mechanical injury.

107. The method of any one of claims 73-78, wherein the fibrosis is a secondary fibrotic disease.

108. The method of claim 107, wherein the secondary fibrotic disease is associated with a disease or disorder selected from the group consisting of an infection, an autoimmune disease or disorder, cancer, and an inflammatory disease or disorder.

109. The method of claim 107, wherein the secondary fibrotic disease is associated with a disease or disorder selected from the group consisting of: atherosclerosis, atrial fibrillation, chronic heart failure, peripheral artery disease, acute coronary syndromes, non-alcoholic fatty liver disease (NAFLD), acute-on-chronic liver failure, acute liver failure, acute kidney injury, acute tubular necrosis, and chronic kidney disease.

110. The method of claim 108, wherein the infection is selected from the group consisting of: sepsis, an HIV infection, a SARS-CoV-2 infection, acute viral hepatitis, chronic viral hepatitis, and malaria.

111. The method of claim 108, wherein the autoimmune or inflammatory disease or disorder or is selected from the group consisting of: acute lung injury (ALI), acute respiratory distress syndrome (ARDS), hypersensitivity pneumonitis, alcoholic hepatitis, non-alcoholic steatohepatitis, viral hepatitis, sickle cell disease, Type I diabetes mellitus, Type 2 diabetes mellitus, Crohn's disease, celiac disease, asthma, sarcoidosis, glomerulonephritis, lupus nephritis, systemic lupus erythematosus, rheumatoid arthritis, Sjögren's Syndrome, scleroderma, cystic fibrosis (CF), graft-versus-host disease, allograft rejection, kidney allograft rejection, sarcoidosis, pulmonary sarcoidosis, hemophagocytic lymphohistiocytosis (HLH), inflammatory arthritis, chronic obstructive pulmonary disease (COPD), asthma, osteoarthritis, fibroids, and multiple sclerosis.

112. The method according to any one of claims 73 and 95-111, further comprising administering to the subject an anti-inflammatory therapy.

113. A pharmaceutical composition, comprising (a) an antibody according to any one of claims 1-72, and (b) a pharmaceutically acceptable excipient.

114. The pharmaceutical composition of claim 113, wherein the pharmaceutically acceptable excipient is selected from the group consisting of a stabilizer, buffer, surfactant, filler, solvent, tonicity or osmolarity adjusting agent, and antioxidant.

115. The pharmaceutical composition of claim 114, comprising two or more pharmaceutically acceptable excipients independently selected from the group consisting of stabilizers, buffers, surfactants, fillers, solvents, tonicity agents, and antioxidants.

116. An antibody according to any one of claims 1-72 for use as a medicament.

117. An antibody according to any one of claims 1-72 for use in treating a cancer or a fibrotic disease or disorder associated with a presence of M2-macrophages in a subject in need thereof.

118. The antibody for use according to claim 116 or 117, wherein binding of the antibody to a macrophage promotes an immune cell function as measured by one or both of the following parameters:
(a) activation of a CD4+ T cell, CD8+ T cell, NK cell, or any combination thereof: and
(b) proliferation of a CD4+ T cell, CD8+ T cell, NK cell, or any combination thereof.

119. The antibody for use according to claim 118, wherein the activation of a CD4+ T cell, CD8+ T cell, NK cell, or any combination thereof is measured as an enhanced level of IFN-γ, TNF-α, or perforin, or any combination thereof.

120. The antibody for use according to any one of claims 116-119, wherein binding of the antibody to a macrophage is not cytotoxic to the macrophage.

121. The antibody for use according to any one of claims 116-120, wherein binding of the antibody to a macrophage results in at least one of the following effects:
(a) reduced expression of at least one marker by the macrophage, wherein the at least one marker is CD16, CD64, TLR2, or Siglec-15;
(b) internalization of the antibody by the macrophage;
(c) secretion of IFN-γ, TNF-α, and perforin;
(d) activation of a CD4+ T cell, CD8+ T cell, NK cell, or any combination thereof:
(e) proliferation of a CD4+ T cell, CD8+ T cell, NK cell, or any combination thereof: and
(f) promotion of tumor cell killing in a tumor microenvironment.

122. The antibody for use according to claim 121, wherein the binding results in: two or more of (a) through (e); three or more of (a) through (e); four or more of (a) through (e); or all of (a) through (e).

123. The antibody for use according to any one of claims 116-122, wherein binding of the antibody to a macrophage increases an immunostimulatory activity in a tumor microenvironment.

124. The antibody for use according to any one of claims 116-123, wherein binding of the antibody to a macrophage reduces an immunosuppression activity of the macrophage.

125. The antibody for use according to any one of claims 116-124, wherein binding of the antibody to a macrophage reduces a tumor promoting activity of the macrophage.

126. The antibody for use according to any one of claims 116-125, wherein binding of the antibody promotes CD4+ T cell activation, CD4+ T cell proliferation, or both CD4+ T cell activation and proliferation.

127. The antibody for use according to any one of claims 116-126, wherein binding promotes expression of CD69, ICOS, OX40, PD1, LAG3, CTLA4, or any combination thereof by CD4+ T cells.

128. The antibody for use according to any one of claims 116-127, wherein binding to the antibody promotes CD8+ T cell activation, CD8+ T cell proliferation, or both CD8+ T cell activation and proliferation.

129. The antibody for use according to any one of claims 116-128, wherein binding of the antibody promotes expression of ICOS, OX40, PD1, LAG3, CTLA4, or any combination thereof by CD8+ T cells.

130. The antibody for use according to any one of claims 116-129, wherein binding of the antibody protein promotes cytotoxic lymphocyte-mediated killing of cancer cells.

131. The antibody for use according to any one of claims 116-130, wherein binding of the antibody promotes NK cell-mediated tumor cell killing.

132. The antibody for use according to any one of claims 116-131, wherein binding of the antibody promotes expression of IL-2 by T cells.

133. The antibody for use according to any one of claims 116-132, wherein binding of the antibody increases CD4+ T cells, CD196− T cells, CXCR3+ T cells, CCR4− T cells, or any combination thereof.

134. The antibody for use according to any one of claims 116-133, wherein binding of the antibody to a macrophage reduces suppression of cytotoxic T cell-mediated killing of tumor cells in the tumor microenvironment.

135. The antibody for use according to any one of claims 117-134, wherein the cancer is a lung cancer.

136. The antibody for use according to any one of claims 117-135, wherein the cancer is a lung carcinoma or a lung sarcoma.

137. The antibody for use according to any one of claims 117-136, wherein the cancer is a lung adenocarcinoma.

138. The antibody for use according to any one of claims 117-137, wherein the antibody is formulated for administration to the subject in combination with an anti-cancer therapeutic.

139. The antibody for use according to any one of claims 117-119, wherein binding of the antibody to a macrophage reduces the pro-fibrotic function of the macrophage.

140. The antibody for use according to any one of claims 117-120, wherein the fibrotic disease or disorder is lung fibrosis.

141. The antibody for use according to any one of claims 117-120, wherein the fibrotic disease or disorder is cardiac fibrosis.

142. The antibody for use according to any one of claims 117-120, wherein the fibrotic disease or disorder is hepatic fibrosis.

143. The antibody for use according to any one of claims 117-120, wherein the fibrotic disease or disorder is renal fibrosis.

144. The antibody for use according to any one of claims 117-120, wherein the fibrotic disease or disorder is retinal fibrosis.

145. The antibody for use according to any one of claims 117-120, wherein the fibrosis is a primary fibrotic disease or disorder.

146. The antibody for use according to claim 145, wherein the primary fibrotic disease or disorder is idiopathic pulmonary fibrosis (IPF).

147. The antibody for use according to claim 145, wherein the primary fibrotic disease or disorder is hepatic cirrhosis.

148. The antibody for use according to claim 145, wherein the primary fibrotic disease or disorder is systemic sclerosis.

149. The antibody for use according to claim 145, wherein the primary fibrotic disease or disorder is radiation fibrosis.

150. The antibody for use according to claim 145, wherein the primary fibrotic disease or disorder is scarring associated with a mechanical injury.

151. The antibody for use according to any one of claims 117-120, wherein the fibrosis is a secondary fibrotic disease.

152. The antibody for use according to claim 151, wherein the secondary fibrotic disease is associated with a disease or disorder selected from the group consisting of an infection, an autoimmune disease or disorder, cancer, and an inflammatory disease or disorder.

153. The antibody for use according to claim 151, wherein the secondary fibrotic disease is associated with a disease or disorder selected from the group consisting of: atherosclerosis, atrial fibrillation, chronic heart failure, peripheral artery disease, acute coronary syndromes, non-alcoholic fatty liver disease (NAFLD), acute-on-chronic liver failure, acute liver failure, acute kidney injury, acute tubular necrosis, and chronic kidney disease.

154. The antibody for use according to claim 152, wherein the infection is selected from the group consisting of: sepsis, an HIV infection, a SARS-CoV-2 infection, acute viral hepatitis, chronic viral hepatitis, and malaria.

155. The antibody for use according to claim 152, wherein the autoimmune or inflammatory disease or disorder or is selected from the group consisting of: acute lung injury (ALI), acute respiratory distress syndrome (ARDS), hypersensitivity pneumonitis, alcoholic hepatitis, non-alcoholic steatohepatitis, viral hepatitis, sickle cell disease, Type 1 diabetes mellitus, Type 2 diabetes mellitus, Crohn's disease, celiac disease, asthma, sarcoidosis, glomerulonephritis, lupus nephritis, systemic lupus erythematosus, rheumatoid arthritis, Sjögren's Syndrome, scleroderma, cystic fibrosis (CF), graft-versus-host disease, allograft rejection, kidney allograft rejection, sarcoidosis, pulmonary sarcoidosis, hemophagocytic lymphohistiocytosis (HLH), inflammatory arthritis, chronic obstructive pulmonary disease (COPD), asthma, osteoarthritis, fibroids, and multiple sclerosis.

156. The method according to any one of claims 116 and 139-155, wherein the antibody is formulated for administration to the subject in combination with an anti-inflammatory therapy.


SEQUENCE LISTING (46 sequences)

SEQ ID NO: 1 - protein, 11 aa, Artificial Sequence
  source: /note=“Description of Artificial Sequence Synthetic peptide”
RASQSISSYLN

SEQ ID NO: 2 - protein, 7 aa, Artificial Sequence
  source: /note=“Description of Artificial Sequence Synthetic peptide”
AASSLQS

SEQ ID NO: 3 - protein, 10 aa, Artificial Sequence
  source: /note=“Description of Artificial Sequence Synthetic peptide”
QQSYSTPRGT

SEQ ID NO: 4 - protein, 5 aa, Artificial Sequence
  source: /note=“Description of Artificial Sequence Synthetic peptide”
SYAMH

SEQ ID NO: 5 - protein, 17 aa, Artificial Sequence
  source: /note=“Description of Artificial Sequence Synthetic peptide”
VISYDGSNKYYADSVKG

SEQ ID NO: 6 - protein, 24 aa, Artificial Sequence
  source: /note=“Description of Artificial Sequence Synthetic peptide”
ENVRPYYDFWSGYYSEYYYYGMDV

SEQ ID NO: 7 - protein, 11 aa, Artificial Sequence
  source: /note=“Description of Artificial Sequence Synthetic peptide”
RASQSISRYLN

SEQ ID NO: 8 - protein, 10 aa, Artificial Sequence
  source: /note=“Description of Artificial Sequence Synthetic peptide”
QQSYSTQRGS

SEQ ID NO: 9 - protein, 7 aa, Artificial Sequence
  source: /note=“Description of Artificial Sequence Synthetic peptide”
AASSLQN

SEQ ID NO: 10 - protein, 10 aa, Artificial Sequence
  source: /note=“Description of Artificial Sequence Synthetic peptide”
QQSYSTTRGS

SEQ ID NO: 11 - protein, 10 aa, Artificial Sequence
  source: /note=“Description of Artificial Sequence Synthetic peptide”
QQSYSTQRGA

SEQ ID NO: 12 - protein, 10 aa, Artificial Sequence
  source: /note=“Description of Artificial Sequence Synthetic peptide”
QQSYSTTGGT

SEQ ID NO: 13 - protein, 11 aa, Artificial Sequence
  source: /note=“Description of Artificial Sequence Synthetic peptide”
  VARIANT (8)..(8): /replace=“R” or “K” or “H”
  SITE (1)..(11): /note=“Variant residues given in the sequence have no preference with respect to those in the annotations for variant positions”
RASQSISSYLN

SEQ ID NO: 14 - protein, 7 aa, Artificial Sequence
  source: /note=“Description of Artificial Sequence Synthetic peptide”
  VARIANT (7)..(7): /replace=“N” or “Q” or “T”
  SITE (1)..(7): /note=“Variant residues given in the sequence have no preference with respect to those in the annotations for variant positions”
AASSLQS

SEQ ID NO: 15 - protein, 10 aa, Artificial Sequence
  source: /note=“Description of Artificial Sequence Synthetic peptide”
  VARIANT (7)..(7): /replace=“Q” or “T” or “S” or “N” or “A” or “G”
  VARIANT (8)..(8): /replace=“G” or “A” or “S”
  VARIANT (10)..(10): /replace=“S” or “A” or “G” or “N”
  SITE (1)..(10): /note=“Variant residues given in the sequence have no preference with respect to those in the annotations for variant positions”
QQSYSTPRGT

SEQ ID NO: 16 - protein, 5 aa, Artificial Sequence
  source: /note=“Description of Artificial Sequence Synthetic peptide”
SYDMH

SEQ ID NO: 17 - protein, 17 aa, Artificial Sequence
  source: /note=“Description of Artificial Sequence Synthetic peptide”
VISEDGSNKYNADSVKG

SEQ ID NO: 18 - protein, 24 aa, Artificial Sequence
  source: /note=“Description of Artificial Sequence Synthetic peptide”
ENVRPYYDFWSGYSSEYYYYGLDV

SEQ ID NO: 19 - protein, 5 aa, Artificial Sequence
  source: /note=“Description of Artificial Sequence Synthetic peptide”
SETMH

SEQ ID NO: 20 - protein, 17 aa, Artificial Sequence
  source: /note=“Description of Artificial Sequence Synthetic peptide”
VISEDGSNKYHADSVKG

SEQ ID NO: 21 - protein, 24 aa, Artificial Sequence
  source: /note=“Description of Artificial Sequence Synthetic peptide”
ENVRPYYDFWSGYNSEYYYYGMDV

SEQ ID NO: 22 - protein, 5 aa, Artificial Sequence
  source: /note=“Description of Artificial Sequence Synthetic peptide”
SYVMH

SEQ ID NO: 23 - protein, 17 aa, Artificial Sequence
  source: /note=“Description of Artificial Sequence Synthetic peptide”
VISEDGSNKYEADSVKG

SEQ ID NO: 24 - protein, 24 aa, Artificial Sequence
  source: /note=“Description of Artificial Sequence Synthetic peptide”
ENVRPYYDFWRGYNSEYYYYGLDV

SEQ ID NO: 25 - protein, 5 aa, Artificial Sequence
  source: /note=“Description of Artificial Sequence Synthetic peptide”
  VARIANT (2)..(2): /replace=“E” or “Q” or “D”
  VARIANT (3)..(3): “D” or “T” or “V” or “S” or “G” or “E”
  SITE (1)..(5): /note=“Variant residues given in the sequence have no preference with respect to those in the annotations for variant positions”
SYAMH

SEQ ID NO: 26 - protein, 17 aa, Artificial Sequence
  source: /note=“Description of Artificial Sequence Synthetic peptide”
  VARIANT (4)..(4): /replace=“E” or “Q” or “D”
  VARIANT (11)..(11): /replace=“N” or “H” or “E” or “D” or “K” or “Q” or “R”
  SITE (1)..(17): /note=“Variant residues given in the sequence have no preference with respect to those in the annotations for variant positions”
VISYDGSNKYYADSVKG

SEQ ID NO: 27 - protein, 24 aa, Artificial Sequence
  source: /note=“Description of Artificial Sequence Synthetic peptide”
  VARIANT (11)..(11): /replace=“R” or “K” or “H”
  VARIANT (14)..(14): /replace=“S” or “N” or “T” or “A” or “Q”
  VARIANT (22)..(22): /replace=“L” or “I” or “V”
  SITE (1)..(24): /note=“Variant residues given in the sequence have no preference with respect to those in the annotations for variant positions”
ENVRPYYDFWSGYYSEYYYYGMDV

SEQ ID NO: 28 - protein, 109 aa, Artificial Sequence
  source: /note=“Description of Artificial Sequence Synthetic polypeptide”
DIQMTQSPSSLSASVGDRVTITCRASQSISRYLNWYQQKPGKAPKLLIYAASSLQSGVPSRFSGSGSGTDFTLTISSLQPEDFATYYCQQSYSTQRGSFGQGTKVEIKR

SEQ ID NO: 29 - protein, 132 aa, Artificial Sequence
  source: /note=“Description of Artificial Sequence Synthetic polypeptide”
EVQLVESGGGVVQPGRSLRLSCAASGFTFSSYDMHWVRQAPGKGLEWVAVISEDGSNKYNADSVKGRFTISRDNSKNTLYLQMNSLRAEDTAVYYCARENVRPYYDFWSGYSSEYYYYGLDVWGQGTTVTVS

SEQ ID NO: 30 - protein, 109 aa, Artificial Sequence
  source: /note=“Description of Artificial Sequence Synthetic polypeptide”
DIQMTQSPSSLSASVGDRVTITCRASQSISRYLNWYQQKPGKAPKLLIYAASSLQNGVPSRFSGSGSGTDFTLTISSLQPEDFATYYCQQSYSTTRGSFGQGTKVEIKR

SEQ ID NO: 31 - protein, 133 aa, Artificial Sequence
  source: /note=“Description of Artificial Sequence Synthetic polypeptide”
EVQLVESGGGVVQPGRSLRLSCAASGFTFSSETMHWVRQAPGKGLEWVAVISEDGSNKYHADSVKGRFTISRDNSKNTLYLQMNSLRAEDTAVYYCARENVRPYYDFWSGYNSEYYYYGMDVWGQGTTVTVSS

SEQ ID NO: 32 - protein, 109 aa, Artificial Sequence
  source: /note=“Description of Artificial Sequence Synthetic polypeptide”
DIQMTQSPSSLSASVGDRVTITCRASQSISRYLNWYQQKPGKAPKLLIYAASSLQSGVPSRFSGSGSGTDFTLTISSLQPEDFATYYCQQSYSTQRGAFGQGTKVEIKR

SEQ ID NO: 33 - protein, 133 aa, Artificial Sequence
  source: /note=“Description of Artificial Sequence Synthetic polypeptide”
EVQLVESGGGVVQPGRSLRLSCAASGFTFSSYVMHWVRQAPGKGLEWVAVISEDGSNKYEADSVKGRFTISRDNSKNTLYLQMNSLRAEDTAVYYCARENVRPYYDFWRGYNSEYYYYGLDVWGQGTTVTVSS

SEQ ID NO: 34 - protein, 109 aa, Artificial Sequence; identical to SEQ ID NO: 28
  source: /note=“Description of Artificial Sequence Synthetic polypeptide”

SEQ ID NO: 35 - protein, 133 aa, Artificial Sequence
  source: /note=“Description of Artificial Sequence Synthetic polypeptide”
EVQLVESGGGVVQPGRSLRLSCAASGFTFSSYVMHWVRQAPGKGLEWVAVISEDGSNKYNADSVKGRFTISRDNSKNTLYLQMNSLRAEDTAVYYCARENVRPYYDFWSGYSSEYYYYGLDVWGQGTTVTVSS

SEQ ID NO: 36 - protein, 109 aa, Artificial Sequence
  source: /note=“Description of Artificial Sequence Synthetic polypeptide”
DIQMTQSPSSLSASVGDRVTITCRASQSISRYLNWYQQKPGKAPKLLIYAASSLQSGVPSRFSGSGSGTDFTLTISSLQPEDFATYYCQQSYSTTRGSFGQGTKVEIKR

SEQ ID NO: 37 - protein, 133 aa, Artificial Sequence
  source: /note=“Description of Artificial Sequence Synthetic polypeptide”
EVQLVESGGGVVQPGRSLRLSCAASGFTFSSYDMHWVRQAPGKGLEWVAVISEDGSNKYNADSVKGRFTISRDNSKNTLYLQMNSLRAEDTAVYYCARENVRPYYDFWRGYNSEYYYYGLDVWGQGTTVTVSS

SEQ ID NO: 38 - protein, 109 aa, Artificial Sequence
  source: /note=“Description of Artificial Sequence Synthetic polypeptide”
DIQMTQSPSSLSASVGDRVTITCRASQSISRYLNWYQQKPGKAPKLLIYAASSLQSGVPSRFSGSGSGTDFTLTISSLQPEDFATYYCQQSYSTTGGTFGQGTKVEIKR

SEQ ID NO: 39 - protein, 133 aa, Artificial Sequence
  source: /note=“Description of Artificial Sequence Synthetic polypeptide”
EVQLVESGGGVVQPGRSLRLSCAASGFTFSSETMHWVRQAPGKGLEWVAVISEDGSNKYNADSVKGRFTISRDNSKNTLYLQMNSLRAEDTAVYYCARENVRPYYDFWSGYSSEYYYYGLDVWGQGTTVTVSS

SEQ ID NO: 40 - protein, 109 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSYSTPRGTFGQGTKVEIKR

SEQ ID NO: 41 - protein, 166 aa, Artificial Sequence
  source: /note=“Description of Artificial Sequence Synthetic polypeptide”
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARENVRPYYDFWSGYYSEYYYYGMDVWGQGTTVTVSSASTKGPSVFPLAPSSKSTSGGTAALGCLVKDYF

SEQ ID NO: 42 - protein, 1121 aa, Homo sapiens
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSGGHSEPH

SEQ ID NO: 43 - protein, 27 aa, Homo sapiens
IGRVNASKGFGHIWLDSVSCQGHEPAI

SEQ ID NO: 44 - protein, 11 aa, Homo sapiens
VVCRQLGCGSA

SEQ ID NO: 45 - protein, 14 aa, Homo sapiens
WDCKNWQWGGLTCD

SEQ ID NO: 46 - protein, 6 aa, Artificial Sequence
  source: /note=“Description of Artificial Sequence Synthetic 6xHis tag”
HHHHHH